(19) United States
(12) Patent Application Publication
(10) Pub. No.: US 2016/0368870 A1
(43) Pub. Date: Dec. 22, 2016

(54) OLEFIN SUBSTITUTED OXINDOLES HAVING AMPK ACTIVITY

(71) Applicant: BOEHRINGER INGELHEIM INTERNATIONAL GMBH, Ingelheim Am Rhein (DE)

(72) Inventors: Jagannath MADANAHALLI RANGANATH RAO, Bangalore (IN); Madhavan GURRAM RANGA, Bangalore (IN); Shanmugam PACHIYAPPAN, Bangalore (IN)

(73) Assignee: Boehringer Ingelheim International GmbH, Ingelheim an Rhein (DE)

(21) Appl. No.: 14/898,785

(22) PCT Filed: Jun. 16, 2014
(86) PCT No.: PCT/EP2014/062561

§ 371 Date: Dec. 16, 2015

(30) Foreign Application Priority Data
Jun. 20, 2013 (IN) 2686/CHE/2013

(51) Int. Cl. C07D 209/34; A61K 31/404; C07D 403/06; A61K 31/4155; C07D 417/06; A61K 31/427; C07D 401/04; A61K 31/4439; C07D 471/04; A61K 31/437; C07D 401/10; C07D 401/14; C07D 403/04; A61K 31/506; C07D 403/10; C07D 403/14; A61K 45/06

(52) U.S. Cl. CPC C07D 209/34; A61K 45/06; A61K 31/404; C07D 403/06; A61K 31/4155; C07D 417/06; A61K 31/427; C07D 401/04; A61K 31/4439; C07D 471/04; A61K 31/437; C07D 401/10; C07D 401/14; C07D 403/04; A61K 31/506; C07D 403/10; C07D 403/14

(57) ABSTRACT

The present invention relates to compounds of formula (I), which have valuable pharmacological properties, in particular are activators of AMPK and which are therefore useful in the treatment of certain disorders that can be prevented or treated by activation of this receptor. The compounds are suitable for treatment and prevention of diseases which can be influenced by this receptor, such as metabolic diseases, in particular diabetes type 2.

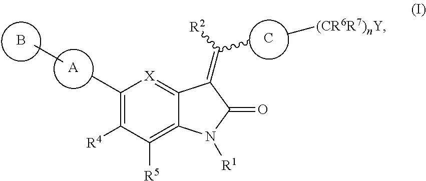

### FIELD OF THE INVENTION

The present invention relates to heterocyclic organic compounds for therapeutic application in human medicine. The present invention more specifically relates to compounds that have the ability to activate 5′ AMP-activated protein kinase (AMPK) which are therefore useful in the treatment of certain disorders that can be prevented or treated by activation of this enzyme. In addition the invention relates to the compounds, methods for their preparation, pharmaceutical compositions containing the compounds and the uses of these compounds in the treatment of certain disorders. It is expected that the compounds of the invention will find application in the treatment of conditions such as non-insulin dependent type 2 diabetes mellitus (NIDDM), insulin resistance, obesity, impaired fasting glucose, impaired glucose tolerance, lipid disorders such as dyslipidemia, hypertension, Cardiovascular diseases, Cancer, Inflammation, trauma and as well as other diseases and conditions.

### BACKGROUND OF THE INVENTION

Metabolic disorders, more specifically Type 2 Diabetes, obesity, cardiovascular diseases that result from both environmental and genetic factors are considered to be some of the fastest growing public health problems globally. These conditions may be associated with reduced insulin action and impaired glucose and lipid metabolism.

AMPK, a heterotrimeric serine/threonine kinase widely recognized as a key regulator of fatty acid and glucose homeostasis is emerging as an attractive target for the treatment of these conditions since it is involved in the regulation of whole body energy metabolism. It not only plays a key role of an energy sensor by sensing intracellular ATP levels, but also acts as a regulator by being a crucial component in maintaining the energy balance within cells. Under conditions of energy depletion, AMPK inhibits ATP-consuming pathways such as fatty acid synthesis, cholesterol synthesis and gluconeogenesis and stimulates ATP-generating processes such as fatty acid oxidation and glycolysis thus restoring the overall cellular energy homeostasis. Through its central role in the regulation of glucose and lipid metabolism, AMPK has become a promising molecular target for the treatment of metabolic disorders. Moreover, the effects of AMPK activation are pleiotropic in key metabolically relevant tissues, such as liver, skeletal muscle, adipose, and hypothalamus.

AMPK is a heterotrimeric enzyme comprised of a catalytic (α1 or α2) subunit and two regulatory (β1 or β2 and γ1, γ2, or γ3) subunits, all of which are encoded by separate genes, making it possible to form a total of 12 complexes (Hardie, “AMPK—the fuel gauge of the eukaryotic cell, the FASEB Journal. 2008; 22:114.1).

A number of physiological processes have been shown to stimulate AMPK, including conditions that lead to alterations of the intracellular AMP/ATP ratio (e.g., hypoxia, glucose deprivation) and calcium concentration, as well as the action of various hormones, cytokines, and adipokines. In mammalian cells, AMPK is activated by increases in intracellular AMP by an allosteric mechanism and by regulating the level of AMPK phosphorylation by inhibiting the dephosphorylation of Thr 172 in the activation loop of the kinase domain (Xiao et al, “Structural basis for AMP binding to mammalian AMP-activated protein kinase”, Nature 496, Vol. 449, September 2007).

The activated form of the enzyme is responsible for metabolic changes via phosphorylation of various downstream substrates. The net effect is a change in local and whole-body energy utilization from an energy consuming state to an energy-producing state in order to restore energy balance. These findings, coupled with reports that AMPK in muscle is activated in response to exercise have led to an intense interest in developing AMPK activators as potential therapies for T2DM and obesity (Zhang, Zhou and Li, “AMPK: An emerging Drug Target for Diabetes and the Metabolic Syndrome”, Cell Metabolism 9, May 6, 2009).

Given the potential broad spectrum of diseases and disorders that can be addressed by activating AMPK, the inventors of this invention have provided a series of oxindole derivatives that activate AMPK and thus can be instrumental in the prophylaxis and treatment of metabolic conditions such as diabetes, obesity, hyperglycemia, glucose intolerance, insulin resistance, hyperinsulemia, hypercholesteremia, hypertension, hyperlipoproteinemia, hyperlipidemia, hypertriglyceridemia, dyslipedemia, metabolic syndrome X, atherosclerosis, diabetic neuropathy, diabetic retinopathy, and hypoglycaemia among others and other disease conditions such as Cancer and Inflammation.

### OBJECTS OF INVENTION

The principal object of the invention is to provide compounds that are activators of 5′ AMP-activated protein kinase. These compounds would be expected to be useful in the treatment of 5′ AMP-activated protein kinase related conditions as discussed above.

Another object is to provide a pharmaceutical composition containing a compound that is an activator of 5′ AMP-activated protein kinase and a pharmaceutically acceptable excipient, diluent or carrier.

A further object is to provide a method of prevention or treatment of a condition associated with 5′ AMP-activated protein kinase activity in a mammal.

### STATEMENT OF INVENTION

The present invention provides compounds of formula (I):

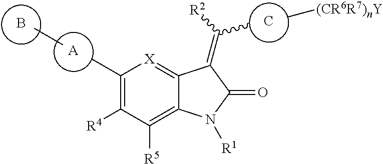

wherein

ring A, ring B and ring C are each independently selected from the group consisting of optionally substituted C₆-C₁₈aryl and optionally substituted C₁-C₁₈heteroaryl;

X is selected from the group consisting of N and CR³;

Y is selected from the group consisting of H and COR⁸

R¹ and R² are each independently selected from the group consisting of H and optionally substituted C₁-C₆ alkyl;

each R³, and R⁵ are each independently selected from the group consisting of H, halogen, CN, —NO₂, SH, CF₃, OH, CO₂H, CONH₂, OCF₃, and optionally substituted C₁-C₁₂alkyl;

R⁴ is selected from the group consisting of H, F, Cl, Br and I;

R⁶ and R⁷ are each independently selected from the group consisting of H and optionally substituted C₁-C₆ alkyl;

R⁸ is selected from the group consisting of H, OH, optionally substituted C₁-C₆ alkyl and —NR⁹R¹⁰;

wherein R⁹ and R¹⁰ are each independently selected from the group consisting of H and optionally substituted C₁-C₆ alkyl, or R⁹ and R¹⁰ when taken together to the nitrogen atom to which they are attached form an optionally substituted C₂-C₁₂ heterocycloalkyl group,

n is an integer selected from the group consisting of 0, 1 and 2;

wherein the term “optionally substituted” used within the definitions hereinbefore is not limited to but preferably means 1, 2 or 3 optional substituents independently selected from F, Cl, Br, I, CH₃, CH₂CH₃, CH(CH₃)₂, C(CH₃)₃, OH, OCH₃, OCH₂CH₃, OCH(CH₃)₂, OC(CH₃)₃, CF₃, OCF₃, NO₂, SO₃H, SO₂CH₃, NH₂, NHCH₃, N(CH₃)₂ and CN;

or a pharmaceutically acceptable salt, N-oxide, or prodrug thereof.

In a further aspect the invention relates to a pharmaceutical composition containing a compound of the invention and a pharmaceutically acceptable diluent, excipient or carrier.

In yet an even further aspect the invention relates to a method of prevention or treatment of a condition associated with 5′ AMP-activated protein kinase activity in a mammal, the method comprising administering an effective amount of a compound of the invention to the mammal.

In yet an even further aspect the invention relates to the use of a compound of the invention in the preparation of a medicament for the prevention or treatment of a condition associated with 5′ AMP-activated protein kinase activity in a mammal.

Examples of conditions that may be treated include cancers, dermatological disorders, respiratory and pulmonary system disorders, metabolic disorders, inflammatory diseases and neurodegenerative diseases.

Examples of cancers include Breast Cancer, Cutaneous T-cell lymphoma (relapsed or refractory cutaneous T-cell lymphoma), Lung cancer, Liver cancer (hepatocellular carcinoma), Kaposi's Sarcoma (AIDS related Kaposi's sarcoma), Cutaneous T-cell lymphoma, Skin cancer (basal cell carcinoma), Non-small cell Lung Cancer, Kidney cancer (advanced renal cell cancer), Gastrointestinal (stomach) cancer (advanced aerodigestive tract cancer), Mesothelioma, and Non-small-cell lung cancer.

Examples of dermatological disorders include Dermatitis (severe chronic hand eczema in adults), Psoriasis (Severe Plaque Psoriasis), Psoriasis (moderate to severe psoriasis) and alopecia.

Examples of respiratory and pulmonary system disorders include Bronchial Metaplasia and Pulmonary Fibrosis (Fibrosis).

Examples of metabolic diseases include Pre diabetes, Type 2 diabetes, Obesity, Hypercholesteriolemia, Hypertriglyceridemia, Hypertension, Dyslipidemia, Liver diseases, NASH, and Atherosclerosis.

Examples of inflammatory disorders include Renal fibrosis, Hepatic diseases such as steatosis, steatohepatitis (alcoholic and non alcoholic), Hepatic fibrosis and cirrhosis, and Experimental autoimmune encephalomyelitis.

An example of a neurodegenerative disorder is Alzheimer's disease.

These and other teachings of the invention are set forth herein.

### DETAILED DESCRIPTION OF THE INVENTION

In this specification a number of terms are used which are well known to a skilled addressee. Nevertheless for the purposes of clarity a number of terms will be defined.

As used herein, the term “unsubstituted” means that there is no substituent or that the only substituents are hydrogen.

The term “optionally substituted” as used throughout the specification denotes that the group may or may not be further substituted or fused (so as to form a condensed polycyclic system), with one or more non-hydrogen substituent groups. In certain embodiments the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)R^{e}, —C(═O)OR^{e}, C(═O)NR^{e}R^{f}, C(═NOH)R^{e}, C(═NR^{e})NR^{f}R^{g}, NR^{e}R^{f}, NR^{e}C(═O)R^{f}, NR^{e}C(═O)OR^{f}, NR^{e}C(═O)NR^{f}R^{g}, NR^{e}C(═NR^{f})NR^{g}R^{h}, NR^{e}SO₂R^{f}, —SR^{e}, SO₂NR^{e}R^{f}, —OR^{e}, OC(═O)NR^{e}R^{f}, OC(═O)R^{e} and acyl,

wherein R^{e}, R^{f}, R^{g} and R^{h} are each independently selected from the group consisting of H, C₁-C₁₂alkyl, C₁-C₁₂haloalkyl, C₂-C₁₂alkenyl, C₂-C₁₂alkynyl, C₁-C₁₀heteroalkyl, C₃-C₁₂cycloalkyl, C₃-C₁₂cycloalkenyl, C₁-C₁₂heterocycloalkyl, C₁-C₁₂heterocycloalkenyl, C₆-C₁₈aryl, C₁-C₁₈heteroaryl, and acyl, or any two or more of R^{a}, R^{b}, R^{c} and R^{d}, when taken together with the atoms to which they are attached form a heterocyclic ring system with 3 to 12 ring atoms.

In some embodiments each optional substituent is independently selected from the group consisting of: halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonyl, alkylsulfonyl, arylsulfonyl, aminosulfonyl, aminoalkyl, —COOH, —SH, and acyl.

Examples of particularly suitable optional substituents include F, Cl, Br, I, CH₃, CH₂CH₃, CH(CH₃)₂, C(CH₃)₃, OH, OCH₃, OCH₂CH₃, OCH(CH₃)₂, OC(CH₃)₃, CF₃, OCF₃, NO₂, SO₃H, SO₂CH₃, NH₂, NHCH₃, N(CH₃)₂ and CN.

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group. The expressions linker, linking moiety and bridging group are interchangeably used herein.

“Acyl” means an R—C(═O)— group in which the R group may be an alkyl, cycloalkyl, heterocycloalkyl, aryl or heteroaryl group as defined herein. Examples of acyl include acetyl and benzoyl. The group may be a terminal group or a bridging group. If the group is a terminal group it is bonded to the remainder of the molecule through the carbonyl carbon.

“Acylamino” means an R—C(═O)—NH— group in which the R group may be an alkyl, cycloalkyl, heterocycloalkyl, aryl or heteroaryl group as defined herein. The group may be a terminal group or a bridging group. If the group is a terminal group it is bonded to the remainder of the molecule through the nitrogen atom.

“Alkenyl” as a group or part of a group denotes an aliphatic hydrocarbon group containing at least one carbon-carbon double bond and which may be straight or branched preferably having 2-12 carbon atoms, more preferably 2-10 carbon atoms, most preferably 2-6 carbon atoms, in the normal chain. The group may contain a plurality of double bonds in the normal chain and the orientation about each is independently E or Z. The alkenyl group is preferably a 1-alkenyl group. Exemplary alkenyl groups include, but are not limited to, ethenyl, propenyl, butenyl, pentenyl, hexenyl, heptenyl, octenyl and nonenyl. The group may be a terminal group or a bridging group.

“Alkenyloxy” refers to an alkenyl-O— group in which alkenyl is as defined herein. Preferred alkenyloxy groups are C₁-C₆ alkenyloxy groups. The group may be a terminal group or a bridging group. If the group is a terminal group it is bonded to the remainder of the molecule through the oxygen atom.

“Alkyl” as a group or part of a group refers to a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkylamino” includes both mono-alkylamino and dialkylamino, unless specified. “Mono-alkylamino” means an Alkyl-NH— group, in which alkyl is as defined herein. “Dialkylamino” means a (alkyl)₂N— group, in which each alkyl may be the same or different and are each as defined herein for alkyl. The alkyl group is preferably a C₁-C₆alkyl group. The group may be a terminal group or a bridging group. If the group is a terminal group it is bonded to the remainder of the molecule through the nitrogen atom.

“Alkylaminocarbonyl” refers to a group of the formula (Alkyl)ₓ(H)_{y}NC(═O)— in which alkyl is as defined herein, x is 1 or 2, and the sum of X+Y=2. The group may be a terminal group or a bridging group. If the group is a terminal group it is bonded to the remainder of the molecule through the carbonyl carbon.

“Alkyloxy” refers to an alkyl-O— group in which alkyl is as defined herein. Preferably the alkyloxy is a C₁-C₆alkyloxy. Examples include, but are not limited to, methoxy and ethoxy. The group may be a terminal group or a bridging group.

“Alkyloxyalkyl” refers to an alkyloxy-alkyl-group in which the alkyloxy and alkyl moieties are as defined herein. The group may be a terminal group or a bridging group. If the group is a terminal group it is bonded to the remainder of the molecule through the alkyl group.

“Alkyloxyaryl” refers to an alkyloxy-aryl-group in which the alkyloxy and aryl moieties are as defined herein. The group may be a terminal group or a bridging group. If the group is a terminal group it is bonded to the remainder of the molecule through the aryl group.

“Alkyloxycarbonyl” refers to an alkyl-O—C(═O)— group in which alkyl is as defined herein. The alkyl group is preferably a C₁-C₆ alkyl group. Examples include, but are not limited to, methoxycarbonyl and ethoxycarbonyl. The group may be a terminal group or a bridging group. If the group is a terminal group it is bonded to the remainder of the molecule through the carbonyl carbon.

“Alkyloxycycloalkyl” refers to an alkyloxy-cycloalkyl-group in which the alkyloxy and cycloalkyl moieties are as defined herein. The group may be a terminal group or a bridging group. If the group is a terminal group it is bonded to the remainder of the molecule through the cycloalkyl group.

“Alkyloxyheteroaryl” refers to an alkyloxy-heteroaryl-group in which the alkyloxy and heteroaryl moieties are as defined herein. The group may be a terminal group or a bridging group. If the group is a terminal group it is bonded to the remainder of the molecule through the heteroaryl group.

“Alkyloxyheterocycloalkyl” refers to an alkyloxy-heterocycloalkyl-group in which the alkyloxy and heterocycloalkyl moieties are as defined herein. The group may be a terminal group or a bridging group. If the group is a terminal group it is bonded to the remainder of the molecule through the heterocycloalkyl group.

“Alkylsulfinyl” means an alkyl-S—(═O)— group in which alkyl is as defined herein. The alkyl group is preferably a C₁-C₆ alkyl group. Exemplary alkylsulfinyl groups include, but not limited to, methylsulfinyl and ethylsulfinyl. The group may be a terminal group or a bridging group. If the group is a terminal group it is bonded to the remainder of the molecule through the sulfur atom.

“Alkylsulfonyl” refers to an alkyl-S(═O)₂— group in which alkyl is as defined above. The alkyl group is preferably a C₁-C₆alkyl group. Examples include, but not limited to methylsulfonyl and ethylsulfonyl. The group may be a terminal group or a bridging group. If the group is a terminal group it is bonded to the remainder of the molecule through the sulfur atom.

“Alkynyl” as a group or part of a group means an aliphatic hydrocarbon group containing a carbon-carbon triple bond and which may be straight or branched preferably having from 2-12 carbon atoms, more preferably 2-10 carbon atoms, more preferably 2-6 carbon atoms in the normal chain. Exemplary structures include, but are not limited to, ethynyl and propynyl. The group may be a terminal group or a bridging group.

“Alkynyloxy” refers to an alkynyl-O— group in which alkynyl is as defined herein. Preferred alkynyloxy groups are C₁-C₆alkynyloxy groups. The group may be a terminal group or a bridging group. If the group is a terminal group it is bonded to the remainder of the molecule through the oxygen atom.

“Aminoalkyl” means an NH₂-alkyl-group in which the alkyl group is as defined herein. The group may be a terminal group or a bridging group. If the group is a terminal group it is bonded to the remainder of the molecule through the alkyl group.

“Aminosulfonyl” means an NH₂—S(═O)₂— group. The group may be a terminal group or a bridging group. If the group is a terminal group it is bonded to the remainder of the molecule through the sulfur atom.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇cycloalkyl or C₅₋₇cycloalkenyl group are fused together to form a cyclic structure, such as tetrahydronaphthyl, indenyl or indanyl. The group may be a terminal group or a bridging group. Typically an aryl group is a C₆-C₁₈ aryl group.

“Arylalkenyl” means an aryl-alkenyl-group in which the aryl and alkenyl are as defined herein. Exemplary arylalkenyl groups include phenylallyl. The group may be a terminal group or a bridging group. If the group is a terminal group it is bonded to the remainder of the molecule through the alkenyl group.

“Arylalkyl” means an aryl-alkyl-group in which the aryl and alkyl moieties are as defined herein. Preferred arylalkyl groups contain a C₁₋₅alkyl moiety. Exemplary arylalkyl groups include benzyl, phenethyl, 1-naphthalenemethyl and 2-naphthalenemethyl. The group may be a terminal group or a bridging group. If the group is a terminal group it is bonded to the remainder of the molecule through the alkyl group.

“Arylalkyloxy” refers to an aryl-alkyl-O— group in which the alkyl and aryl are as defined herein. The group may be a terminal group or a bridging group. If the group is a terminal group it is bonded to the remainder of the molecule through the oxygen atom.

“Arylamino” includes both mono-arylamino and di-arylamino unless specified. Mono-arylamino means a group of formula arylNH—, in which aryl is as defined herein. Di-arylamino means a group of formula (aryl)₂N— where each aryl may be the same or different and are each as defined herein for aryl. The group may be a terminal group or a bridging group. If the group is a terminal group it is bonded to the remainder of the molecule through the nitrogen atom.

“Arylheteroalkyl” means an aryl-heteroalkyl-group in which the aryl and heteroalkyl moieties are as defined herein. The group may be a terminal group or a bridging group. If the group is a terminal group it is bonded to the remainder of the molecule through the heteroalkyl group.

“Aryloxy” refers to an aryl-O— group in which the aryl is as defined herein. Preferably the aryloxy is a C₆-C₁₈aryloxy, more preferably a C₆-C₁₀aryloxy. The group may be a terminal group or a bridging group. If the group is a terminal group it is bonded to the remainder of the molecule through the oxygen atom.

“Arylsulfonyl” means an aryl-S(═O)₂— group in which the aryl group is as defined herein. The group may be a terminal group or a bridging group. If the group is a terminal group it is bonded to the remainder of the molecule through the sulfur atom.

A “bond” is a linkage between atoms in a compound or molecule. The bond may be a single bond, a double bond, or a triple bond.

“Cycloalkenyl” means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl. The cycloalkenyl group may be substituted by one or more substituent groups. A cycloalkenyl group typically is a C₃-C₁₂ alkenyl group. The group may be a terminal group or a bridging group.

“Cycloalkyl” refers to a saturated monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₁₂ alkyl group. The group may be a terminal group or a bridging group.

“Cycloalkylalkyl” means a cycloalkyl-alkyl-group in which the cycloalkyl and alkyl moieties are as defined herein. Exemplary monocycloalkylalkyl groups include cyclopropylmethyl, cyclopentylmethyl, cyclohexylmethyl and cycloheptylmethyl. The group may be a terminal group or a bridging group. If the group is a terminal group it is bonded to the remainder of the molecule through the alkyl group.

“Cycloalkylalkenyl” means a cycloalkyl-alkenyl-group in which the cycloalkyl and alkenyl moieties are as defined herein. The group may be a terminal group or a bridging group. If the group is a terminal group it is bonded to the remainder of the molecule through the alkenyl group.

“Cycloalkylheteroalkyl” means a cycloalkyl-heteroalkyl-group in which the cycloalkyl and heteroalkyl moieties are as defined herein. The group may be a terminal group or a bridging group. If the group is a terminal group it is bonded to the remainder of the molecule through the heteroalkyl group.

“Cycloalkyloxy” refers to a cycloalkyl-O— group in which cycloalkyl is as defined herein. Preferably the cycloalkyloxy is a C₁-C₆cycloalkyloxy. Examples include, but are not limited to, cyclopropanoxy and cyclobutanoxy. The group may be a terminal group or a bridging group. If the group is a terminal group it is bonded to the remainder of the molecule through the oxygen atom.

“Cycloalkenyloxy” refers to a cycloalkenyl-O— group in which the cycloalkenyl is as defined herein. Preferably the cycloalkenyloxy is a C₁-C₆cycloalkenyloxy. The group may be a terminal group or a bridging group. If the group is a terminal group it is bonded to the remainder of the molecule through the oxygen atom.

“Haloalkyl” refers to an alkyl group as defined herein in which one or more of the hydrogen atoms has been replaced with a halogen atom selected from the group consisting of fluorine, chlorine, bromine and iodine. A haloalkyl group typically has the formula CₙH₍₂ₙ₊₁₋ₘ₎Xₘ wherein each X is independently selected from the group consisting of F, Cl, Br and I. In groups of this type n is typically from 1 to 10, more preferably from 1 to 6, most preferably 1 to 3. m is typically 1 to 6, more preferably 1 to 3. Examples of haloalkyl include fluoromethyl, difluoromethyl and trifluoromethyl.

“Haloalkenyl” refers to an alkenyl group as defined herein in which one or more of the hydrogen atoms has been replaced with a halogen atom independently selected from the group consisting of F, Cl, Br and I.

“Haloalkynyl” refers to an alkynyl group as defined herein in which one or more of the hydrogen atoms has been replaced with a halogen atom independently selected from the group consisting of F, Cl, Br and I.

“Halogen” represents chlorine, fluorine, bromine or iodine.

“Heteroalkyl” refers to a straight- or branched-chain alkyl group preferably having from 2 to 12 carbons, more preferably 2 to 6 carbons in the chain, in which one or more of the carbon atoms (and any associated hydrogen atoms) are each independently replaced by a heteroatomic group selected from S, O, P and NR′ where R′ is selected from the group consisting of H, optionally substituted C₁-C₁₂alkyl, optionally substituted C₃-C₁₂cycloalkyl, optionally substituted C₆-C₁₈aryl, and optionally substituted C₁-C₁₈heteroaryl. Exemplary heteroalkyls include alkyl ethers, secondary and tertiary alkyl amines, amides, alkyl sulfides, and the like. Examples of heteroalkyl also include hydroxyC₁-C₆alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl. The group may be a terminal group or a bridging group.

“Heteroalkyloxy” refers to a heteroalkyl-O— group in which heteroalkyl is as defined herein. Preferably the heteroalkyloxy is a C₂-C₆heteroalkyloxy. The group may be a terminal group or a bridging group.

“Heteroaryl” either alone or part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. The group may be a monocyclic or bicyclic heteroaryl group. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. A heteroaryl group is typically a C₁-C₁₈heteroaryl group. The group may be a terminal group or a bridging group.

“Heteroarylalkyl” means a heteroaryl-alkyl group in which the heteroaryl and alkyl moieties are as defined herein. Preferred heteroarylalkyl groups contain a lower alkyl moiety. Exemplary heteroarylalkyl groups include pyridylmethyl. The group may be a terminal group or a bridging group. If the group is a terminal group it is bonded to the remainder of the molecule through the alkyl group.

“Heteroarylalkenyl” means a heteroaryl-alkenyl-group in which the heteroaryl and alkenyl moieties are as defined herein. The group may be a terminal group or a bridging group. If the group is a terminal group it is bonded to the remainder of the molecule through the alkenyl group.

“Heteroarylheteroalkyl” means a heteroaryl-heteroalkyl-group in which the heteroaryl and heteroalkyl moieties are as defined herein. The group may be a terminal group or a bridging group. If the group is a terminal group it is bonded to the remainder of the molecule through the heteroalkyl group.

“Heteroaryloxy” refers to a heteroaryl-O— group in which the heteroaryl is as defined herein. Preferably the heteroaryloxy is a C₁-C₁₈heteroaryloxy. The group may be a terminal group or a bridging group. If the group is a terminal group it is bonded to the remainder of the molecule through the oxygen atom.

“Heterocyclic” refers to saturated, partially unsaturated or fully unsaturated monocyclic, bicyclic or polycyclic ring system containing at least one heteroatom selected from the group consisting of nitrogen, sulfur and oxygen as a ring atom. Examples of heterocyclic moieties include heterocycloalkyl, heterocycloalkenyl and heteroaryl.

“Heterocycloalkenyl” refers to a heterocycloalkyl group as defined herein but containing at least one double bond. A heterocycloalkenyl group typically is a C₂-C₁₂heterocycloalkenyl group. The group may be a terminal group or a bridging group.

“Heterocycloalkyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. A heterocycloalkyl group typically is a C₂-C₁₂heterocycloalkyl group. The group may be a terminal group or a bridging group.

“Heterocycloalkylalkyl” refers to a heterocycloalkyl-alkyl-group in which the heterocycloalkyl and alkyl moieties are as defined herein. Exemplary heterocycloalkylalkyl groups include (2-tetrahydrofuryl)methyl, (2-tetrahydrothiofuranyl) methyl. The group may be a terminal group or a bridging group. If the group is a terminal group it is bonded to the remainder of the molecule through the alkyl group.

“Heterocycloalkylalkenyl” refers to a heterocycloalkyl-alkenyl-group in which the heterocycloalkyl and alkenyl moieties are as defined herein. The group may be a terminal group or a bridging group. If the group is a terminal group it is bonded to the remainder of the molecule through the alkenyl group.

“Heterocycloalkylheteroalkyl” means a heterocycloalkyl-heteroalkyl-group in which the heterocycloalkyl and heteroalkyl moieties are as defined herein. The group may be a terminal group or a bridging group. If the group is a terminal group it is bonded to the remainder of the molecule through the heteroalkyl group.

“Heterocycloalkyloxy” refers to a heterocycloalkyl-O— group in which the heterocycloalkyl is as defined herein. Preferably the heterocycloalkyloxy is a C₁-C₆heterocycloalkyloxy. The group may be a terminal group or a bridging group. If the group is a terminal group it is bonded to the remainder of the molecule through the oxygen atom.

“Heterocycloalkenyloxy” refers to a heterocycloalkenyl-O— group in which heterocycloalkenyl is as defined herein. Preferably the Heterocycloalkenyloxy is a C₁-C₆ Heterocycloalkenyloxy. The group may be a terminal group or a bridging group. If the group is a terminal group it is bonded to the remainder of the molecule through the oxygen atom.

“Hydroxyalkyl” refers to an alkyl group as defined herein in which one or more of the hydrogen atoms has been replaced with an OH group. A hydroxyalkyl group typically has the formula CₙH₍₂ₙ₊₁₋ₓ₎(OH)ₓ. In groups of this type n is typically from 1 to 10, more preferably from 1 to 6, most preferably 1 to 3. x is typically 1 to 6, more preferably 1 to 3.

“Sulfinyl” means an R—S(═O)— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfinylamino” means an R—S(═O)—NH— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the nitrogen atom.

“Sulfonyl” means an R—S(═O)₂— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfonylamino” means an R—S(═O)₂—NH— group. The group may be a terminal group or a bridging group. If the group is a terminal group it is bonded to the remainder of the molecule through the nitrogen atom.

The term “prodrug” as used herein refers to (i) an inactive form of a drug that exerts its effects after metabolic processes within the body converting it to a usable or active form, or (ii) a substance that gives rise to a pharmacologically active metabolite, although not itself active (i.e. an inactive precursor). The terms “prodrug” or “prodrug derivative” mean a covalently-bonded derivative, carrier or precursor of the parent compound or active drug substance which undergoes at least some biotransformation prior to exhibiting its pharmacological effect(s). Such prodrugs either have metabolically cleavable or otherwise convertible groups and are rapidly transformed in vivo to yield the parent compound, for example, by hydrolysis in blood or by activation via oxidation as in case of thioether groups. Most common prodrugs include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309-396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; Bioreversible Carriers in Drug Design, E. B. Roche (ed.), Elsevier, 1987, each of which is incorporated herein by reference in their entireties.

It is understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art. For those compounds where there is the possibility of geometric isomerism the applicant has drawn the isomer that the compound is thought to be although it will be appreciated that the other isomer may be the correct structural assignment. Where the structural isomer is not known or where the compound is thought to be a mixture of the two isomers the attachment to the double bond is shown as a wavy line.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, Formula (I) is intended to cover, where applicable, solvated as well as unsolvated forms of the compounds. Thus, each formula includes compounds having the indicated structure, including the hydrated as well as the non-hydrated forms.

The term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propanoic, succinic, glycolic, gluconic, lactic, malic, tartaric, citric, fumaric, maleic, alkyl sulfonic, arylsulfonic. In a similar vein base addition salts may be prepared by ways well known in the art using organic or inorganic bases. Example of suitable organic bases include simple amines such as methylamine, ethylamine, triethylamine and the like. Examples of suitable inorganic bases include NaOH, KOH, and the like. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

As stated above the compounds of the invention have the formula:

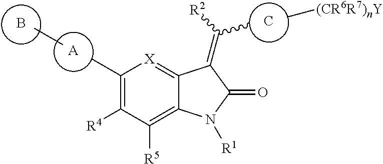

As with any group of structurally related compounds which possess a particular utility, certain embodiments of variables of the compounds of the Formula (I), are particularly useful in their end use application.

In some embodiments Y is H. In some embodiments Y is COR⁸.

In some embodiments R⁸ is H. In certain embodiments R⁸ is NR⁹R¹⁰. In certain embodiments R⁸ is OH.

In certain embodiments of the invention Y is COR⁸, and R⁸ is OH. This provides compounds of formula (Ia).

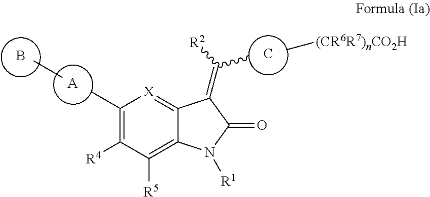

or a pharmaceutically acceptable salt thereof;
wherein ring A, Ring B, Ring C, X, R¹, R², R⁴, R⁵, R⁶, R⁷ and n are as defined above.

In some embodiments of the invention X is N. In some embodiments of the invention X is CR³.

As stated above R³ is selected from the group consisting of H, halogen, CN, —NO₂, SH, CF₃, OH, CO₂H, CONH₂, OCF₃, and optionally substituted C₁-C₁₂alkyl.

In some embodiments R³ is H. In some embodiments R³ is halogen. In some embodiments R³ is CN. In some embodiments R³ is NO₂. In some embodiments R³ is SH. In some embodiments R³ is CF₃. In some embodiments R³ is OH. In some embodiments R³ is CO₂H. In some embodiments R³ is CONH₂. In some embodiments R³ is OCF₃. In some embodiments R³ is C₁-C₁₂alkyl.

In some embodiments Y is COR⁸, R⁸ is OH and X is N. This provides compounds of formula (Ib).

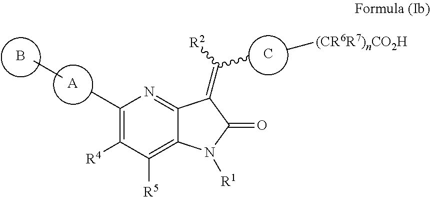

or a pharmaceutically acceptable salt thereof;
wherein ring A, Ring B, Ring C, R¹, R², R⁴, R⁵, R⁶, R⁷ and n are as defined above.

In some embodiments Y is COR⁸, R⁸ is OH, X is CR³ and R³ is H. This provides compounds of formula (Ic).

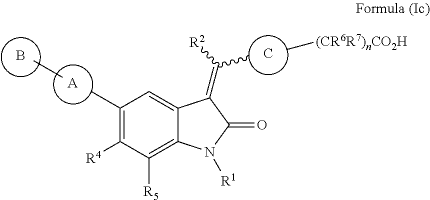

or a pharmaceutically acceptable salt thereof;
wherein ring A, Ring B, Ring C, R¹, R², R⁴, R⁵, R⁶, R⁷ and n are as defined above.

In some embodiments R¹ is H. In some embodiments R¹ is and optionally substituted C₁-C₆ alkyl. In some embodiments R¹ is CH₃. In some embodiments R¹ is CH₂CH₃. In some embodiments R¹ is CH(CH₃)₂. In some embodiments R¹ is C(CH₃)₃.

In some embodiments R² is H. In some embodiments R² is and optionally substituted C₁-C₆ alkyl. In some embodiments R² is CH₃. In some embodiments R² is CH₂CH₃. In some embodiments R² is CH(CH₃)₂. In some embodiments R² is C(CH₃)₃.

In some embodiments R⁵ is H. In some embodiments R⁵ is halogen. In some embodiments R⁵ is CN. In some embodiments R⁵ is NO₂. In some embodiments R⁵ is SH. In some embodiments R⁵ is CF₃. In some embodiments R⁵ is OH. In some embodiments R⁵ is CO₂H. In some embodiments R⁵ is CONH₂. In some embodiments R⁵ is OCF₃. In some embodiments R⁵ is C₁-C₁₂alkyl.

In some embodiments Y is COR⁸, R⁸ is OH, X is CR³, R¹ is H, R² is H, R³ is H and R⁵ is H. This provides compounds of formula (Id).

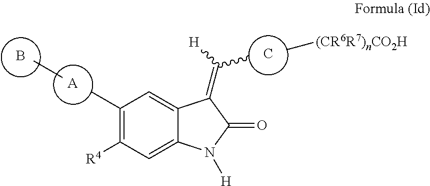

or a pharmaceutically acceptable salt thereof;
wherein ring A, Ring B, Ring C, R⁴, R⁶, R⁷ and n are as defined above.

In the compounds of the invention ring A is selected from the group consisting of optionally substituted C₆-C₁₈aryl and optionally substituted C₁-C₁₈heteroaryl. In certain embodiments ring A is optionally substituted C₆-C₁₈aryl. In certain embodiments ring A is optionally substituted C₁-C₁₈heteroaryl. Ring A may be a monocyclic, bicyclic or polycyclic moiety. In certain embodiments ring A is a monocyclic moiety. In certain embodiments each of ring A is bicyclic moiety.

In certain embodiments ring A is selected from the group consisting of:

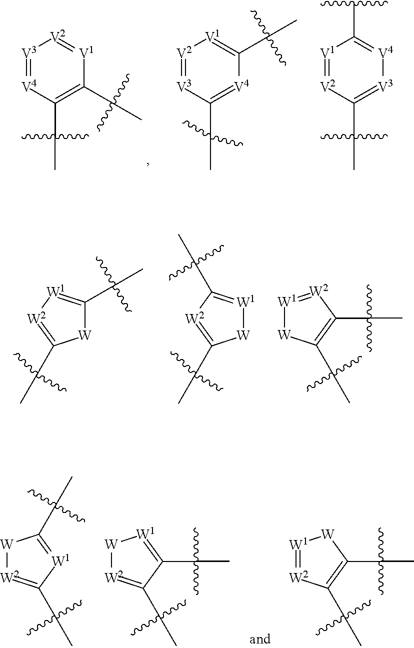

wherein V¹, V², V³ and V⁴ are each independently selected from the group consisting of N, and C(R¹¹);

W is selected from the group consisting of O, S and NR¹¹;

W¹ and W² are each independently selected from the group consisting of N and CR¹¹;

wherein each R¹¹ is independently selected from the group consisting of H, halogen, OH, NO₂, CN, SH, NH₂, CF₃, OCF₃, optionally substituted C₁-C₁₂alkyl, optionally substituted C₁-C₁₂haloalkyl, optionally substituted C₂-C₁₂alkenyl, optionally substituted C₂-C₁₂haloalkenyl optionally substituted C₂-C₁₂alkynyl, optionally substituted C₂-C₁₂haloalkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₂heteroaryloxy, optionally substituted C₁-C₁₂alkylamino, SR¹¹¹ SO₃H, SO₂NR¹¹¹R¹¹², SO₂R¹¹¹, SONR¹¹¹R¹¹², SOR¹¹¹, COR¹¹¹, COOH, COOR¹¹¹, CONR¹¹¹R¹¹², NR¹¹¹COR¹¹², NR¹¹¹COOR¹¹², NR¹¹¹SO₂R¹¹², NR¹¹¹CONR¹¹²R¹¹³, NR¹¹¹R¹¹², and acyl;

each R¹¹¹, R¹¹² and R¹¹³ is independently selected from the group consisting of H, optionally substituted C₁-C₁₂alkyl, optionally substituted C₂-C₁₀heteroalkyl, optionally substituted C₁-C₁₂haloalkyl, optionally substituted C₃-C₁₂cycloalkyl, optionally substituted C₆-C₁₈aryl, and optionally substituted C₁-C₁₈heteroaryl.

Examples of particularly values of R¹¹ include, but are not limited to OH, F, Br, Cl, methyl, CN, NO₂, SH, CO₂H, CONH₂, OCF₃, trifluoromethyl, ethyl, 2,2,2-trifluoroethyl, isopropyl, propyl, 2-ethyl-propyl, 3,3-dimethyl-propyl, butyl, isobutyl, 3,3-dimethyl-butyl, 2-ethyl-butyl, pentyl, 2-methyl-pentyl, pent-4-enyl, hexyl, heptyl, octyl, phenyl, NH₂, phenoxy, hydroxy, methoxy, ethoxy, pyrrol-1-yl, and 3,5-dimethyl-pyrazol-1-yl.

In certain embodiments ring A is an optionally substituted C₆-C₁₈aryl group of the formula (II):

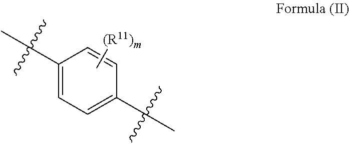

wherein each R¹¹ is independently selected from the group consisting of H, halogen, CN, OH, NH₂, NO₂, SH, CF₃, CO₂H, CONH₂, C₁-C₁₂alkyl, C₁-C₁₂haloalkyl, C₁-C₁₂alkoxyl, and C₁-C₁₂haloalkoxyl,

m is an integer selected from the group consisting of 0, 1, 2, 3, and 4.

In certain embodiments each R¹¹ is independently selected from the group consisting of H, halogen, CN, —NO₂, SH, CF₃, OH, CO₂H, CONH₂, OCF₃, and optionally substituted C₁-C₁₂alkyl;

In the compounds of the invention m is an integer selected from the group consisting of 0, 1, 2, 3 and 4. In some embodiments m is 0. In some embodiments m is 1. In some embodiments m is 2. In some embodiments m is 3. In some embodiments m is 4.

In some embodiments Y is COR⁸, R⁸ is OH, X is CR³, R¹ is H, R² is H, R³ is H, R⁵ is H and ring A is a compound of formula (II). This provides compounds of formula (Ie).

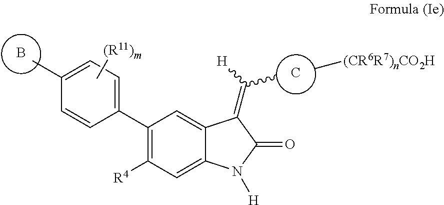

or a pharmaceutically acceptable salt thereof;
wherein Ring B, Ring C, R⁴, R⁶, R⁷, R¹¹, n and m are as defined above.

In the compounds of the invention ring B is selected from the group consisting of optionally substituted C₆-C₁₈aryl and optionally substituted C₁-C₁₈heteroaryl. In certain embodiments ring B is optionally substituted C₆-C₁₈aryl. In certain embodiments ring B is optionally substituted C₁-C₁₈heteroaryl. Ring B may be a monocyclic, bicyclic or polycyclic moiety. In certain embodiments ring B is a monocyclic moiety. In certain embodiments each of ring A is bicyclic moiety.

In certain embodiments ring B is selected from the group consisting of:

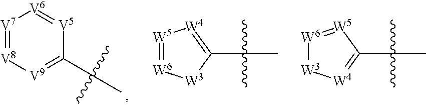

wherein V⁵, V⁶, V⁷, V⁸ and V⁹ are each independently selected from the group consisting of N, and C(R¹²);

W³ is selected from the group consisting of O, S and NR¹²;

W⁴, W⁵, and W⁶ are each independently selected from the group consisting of N and CR¹²;

wherein each R¹² is independently selected from the group consisting of H, halogen, OH, NO₂, CN, SH, NH₂, CF₃, OCF₃, optionally substituted C₁-C₁₂alkyl, optionally substituted C₁-C₁₂haloalkyl, optionally substituted C₂-C₁₂alkenyl, optionally substituted C₂-C₁₂haloalkenyl optionally substituted C₂-C₁₂alkynyl, optionally substituted C₂-C₁₂haloalkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₂heteroaryloxy, optionally substituted C₁-C₁₂alkylamino, SR¹³, SO₃H, SO₂NR¹³R¹⁴, SO₂R¹³, SONR¹³R¹⁴, SOR¹³, COR¹⁴, COOH, COOR¹³, CONR¹⁴R¹⁵, NR¹⁴COR¹⁵, NR¹⁴COOR¹⁵, NR¹⁴SO₂R¹⁵, NR¹³CONR¹⁴R¹⁵, NR¹⁴R¹⁵, and acyl;

each R¹³, R¹⁴ and R¹⁵ is independently selected from the group consisting of H, optionally substituted C₁-C₁₂alkyl, optionally substituted C₂-C₁₀heteroalkyl, optionally substituted C₁-C₁₂haloalkyl, optionally substituted C₃-C₁₂cycloalkyl, optionally substituted C₆-C₁₈aryl, and optionally substituted C₁-C₁₈heteroaryl.

Examples of particularly values of R¹² include, but are not limited to OH, F, Br, Cl, methyl, CN, NO₂, SH, CO₂H, CONH₂, OCF₃, trifluoromethyl, ethyl, 2,2,2-trifluoroethyl, isopropyl, propyl, 2-ethyl-propyl, 3,3-dimethyl-propyl, butyl, isobutyl, 3,3-dimethyl-butyl, 2-ethyl-butyl, pentyl, 2-methyl-pentyl, pent-4-enyl, hexyl, heptyl, octyl, phenyl, NH₂, phenoxy, hydroxy, methoxy, ethoxy, pyrrol-1-yl, and 3,5-dimethyl-pyrazol-1-yl.

In certain embodiments ring B is an optionally substituted C₆-C₁₈aryl group of the formula (III):

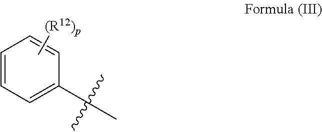

wherein each R¹² is independently selected from the group consisting of H, halogen, OH, NO₂, CN, SH, NH₂, CF₃, OCF₃, optionally substituted C₁-C₁₂alkyl, optionally substituted C₁-C₁₂haloalkyl, optionally substituted C₂-C₁₂alkenyl, optionally substituted C₂-C₁₂haloalkenyl optionally substituted C₂-C₁₂alkynyl, optionally substituted C₂-C₁₂haloalkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₂heteroaryloxy, optionally substituted C₁-C₁₂alkylamino, SR¹³, SO₃H, SO₂NR¹³R¹⁴, SO₂R¹³, SONR¹³R¹⁴, SOR¹³, COR¹⁴, COOH, COOR¹³, CONR¹⁴R¹⁵, NR¹⁴COR¹⁵, NR¹⁴COOR¹⁵, NR¹⁴SO₂R¹⁵, NR¹³CONR¹⁴R¹⁵, NR¹⁴R¹⁵, and acyl;

each R¹³, R¹⁴ and R¹⁵ is independently selected from the group consisting of H, optionally substituted C₁-C₁₂alkyl, optionally substituted C₂-C₁₀heteroalkyl, optionally substituted C₁-C₁₂haloalkyl, optionally substituted C₃-C₁₂cycloalkyl, optionally substituted C₆-C₁₈aryl, and optionally substituted C₁-C₁₈heteroaryl;

p is an integer selected from the group consisting of 0, 1, 2, 3, 4 and 5;

wherein each optional substituent is independently selected from the group consisting of include F, Cl, Br, I, CH₃, CH₂CH₃, CH(CH₃)₂, C(CH₃)₃, OH, OCH₃, OCH₂CH₃, OCH(CH₃)₂, OC(CH₃)₃, CF₃, OCF₃, NO₂, SO₃H, SO₂CH₃, NH₂, NHCH₃, N(CH₃)₂ and CN.

In some embodiments Y is COR⁸, R⁸ is OH, X is CR³, R¹ is H, R² is H, R³ is H, R⁵ is H, ring A is a compound of formula (II) and ring B is a compound of formula (III). This provides compounds of formula (If).

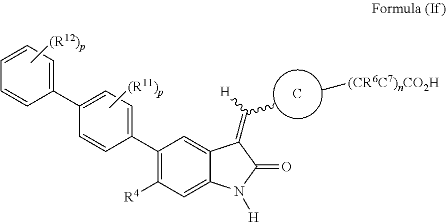

or a pharmaceutically acceptable salt thereof;
wherein Ring C, R⁴, R⁶, R⁷, R¹¹, R¹², m, n and p are as defined above.

In the compounds of the invention p is an integer selected from the group consisting of 0, 1, 2, 3, 4 and 5. In some embodiments p is 0. In some embodiments p is 1. In some embodiments p is 2. In some embodiments p is 3. In some embodiments p is 4. In some embodiments P is 5.

In certain embodiments each R¹² is independently selected from the group consisting of H, halogen, CN, —NO₂, SH, CF₃, OH, CO₂H, CONH₂, OCF₃, and optionally substituted C₁-C₁₂alkyl.

In embodiments where p is 1 the R¹² group may be at any location around the aromatic ring. In certain embodiments the R¹² group is located ortho to the point of attachment to the A ring. In certain embodiment the R¹² group is located meta to the point of attachment to the A ring. In certain embodiments the R¹² group is located para to the point of attachment to the A ring.

In certain embodiments p is 1 and R¹² is OH. In certain embodiments where p is 1 and R¹² is OH, the OH group is located at the ortho position. This provides compounds of formula (Ifa).

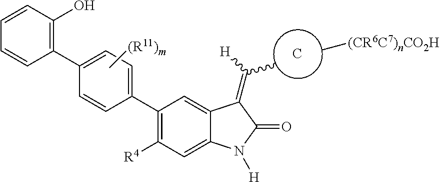

wherein Ring B, Ring C, R⁴, R⁶, R⁷, R¹¹, R¹², m and p are as defined above.

In certain embodiments p is 1 and R¹² is optionally substituted C₁-C₁₈heteroaryl. In certain embodiments R¹² is an optionally substituted C₁-C₁₈heteroaryl selected from the group consisting of:

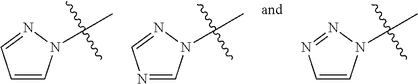 [SMILES: *n1cccn1 ; *n1cncn1 ; *n1ccnn1]

In the compounds of the invention ring C is selected from the group consisting of optionally substituted C₆-C₁₈aryl and optionally substituted C₁-C₁₈heteroaryl. In certain embodiments ring C is optionally substituted C₆-C₁₈aryl. In certain embodiments ring C is optionally substituted C₁-C₁₈heteroaryl. Ring C may be a monocyclic, bicyclic or polycyclic moiety. In certain embodiments ring C is a monocyclic moiety. In certain embodiments each of ring C is bicyclic moiety.

In certain embodiments ring C is selected from the group consisting of:

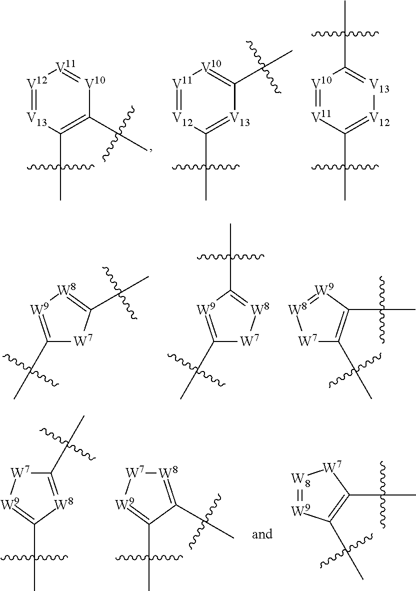

wherein V¹⁰, V¹¹, V¹² and V¹³ are each independently selected from the group consisting of N, and C(R¹⁶);

W⁷ is selected from the group consisting of O, S and NR¹⁶;

W⁸ and W⁹ are each independently selected from the group consisting of N and CR¹⁶;

wherein each R¹⁶ is independently selected from the group consisting of H, halogen, OH, NO₂, CN, SH, NH₂, CF₃, OCF₃, optionally substituted C₁-C₁₂alkyl, optionally substituted C₁-C₁₂haloalkyl, optionally substituted C₂-C₁₂alkenyl, optionally substituted C₂-C₁₂haloalkenyl optionally substituted C₂-C₁₂alkynyl, optionally substituted C₂-C₁₂haloalkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₂heteroaryloxy, optionally substituted C₁-C₁₂alkylamino, SR¹⁶¹ SO₃H, SO₂NR¹⁶¹R¹⁶², SO₂R¹⁶¹, SONR¹⁶¹R¹⁶², SOR¹⁶¹, COR¹⁶¹, COOH, COOR¹⁶¹, CONR¹⁶¹R¹⁶², NR¹⁶¹COR¹⁶², NR¹⁶¹COOR¹⁶², NR¹⁶¹SO₂R¹⁶², NR¹⁶¹CONR¹⁶²R¹⁶³, NR¹⁶¹R¹⁶², and acyl;

each R¹⁶¹, R¹⁶² and R¹⁶³ is independently selected from the group consisting of H, optionally substituted C₁-C₁₂alkyl, optionally substituted C₂-C₁₀heteroalkyl, optionally substituted C₁-C₁₂haloalkyl, optionally substituted C₃-C₁₂cycloalkyl, optionally substituted C₆-C₁₈aryl, and optionally substituted C₁-C₁₈heteroaryl.

In certain embodiments ring C is an optionally substituted C₆-C₁₈aryl group of the formula (IV):

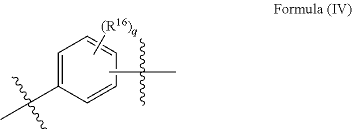

wherein each R¹⁶ is independently selected from the group consisting of H, halogen, CN, —NO₂, SH, CF₃, OH, CO₂H, CONH₂, OCF₃, C₁-C₁₂alkyl and OC₁-C₁₂alkyl;

q is an integer selected from the group consisting of 0, 1, 2, 3, and 4.

In the compounds of the invention q is an integer selected from the group consisting of 0, 1, 2, 3, and 4. In some embodiments q is 0. In some embodiments q is 1. In some embodiments q is 2. In some embodiments q is 3. In some embodiments q is 4.

In some embodiments Y is COR⁸, R⁸ is OH, X is CR³, R¹ is H, R² is H, R³ is H, R⁵ is H, ring A is a compound of formula (II), ring B is a compound of formula (III) and ring C is a compound of formula (IV). This provides compounds of formula (Ig):

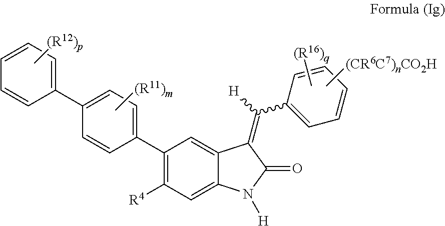

or a pharmaceutically acceptable salt thereof;
wherein R⁴, R⁶, R⁷, R¹¹, R¹², R¹⁶, m, n, p and q are as defined above.

In the compounds of the invention n is an integer selected from the group consisting of 0, 1, and 2. In some embodiments n is 0. In some embodiments n is 1. In some embodiments n is 2.

In the compounds of the invention R⁶ and R⁷ are each independently is selected from the group consisting of H and optionally substituted C₁-C₆ alkyl. In some embodiments R⁶ is H. in some embodiments R⁶ is C₁-C₆ alkyl. In some embodiments R⁶ is methyl. In some embodiments R⁷ is H. In some embodiments R⁷ is C₁-C₆ alkyl. In some embodiments R⁷ is methyl. In some embodiments R⁶ and R⁷ are both H. in some embodiments R⁶ and R⁷ are both C₁-C₆ alkyl. In some embodiments one of R⁶ and R⁷ is H and the other is C₁-C₆ alkyl.

In the compounds of the invention R⁴ is selected from the group consisting of H, F, Cl, Br and I. in some embodiments R⁴ is H. In some embodiments R⁴ is F. In some embodiments R⁴ is Cl. In some embodiments R⁴ is Br. In some embodiments R⁴ is I.

Many if not all of the variables discussed above may be optionally substituted. If the variable is optionally substituted then in some embodiments each optional substituent is independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)R^{e}, —C(═O)OR^{e}, C(═O)NR^{e}R^{f}, C(═NOH)R^{e}, C(═NR^{e})NR^{f}R^{g}, NR^{e}R^{f}, NR^{e}C(═O)R^{f}, NR^{e}C(═O)OR^{f}, NR^{e}C(═O)NR^{f}R^{g}, NR^{e}C(═NR^{f})NR^{g}R^{h}, NR^{e}SO₂R^{f}, —SR^{e}, SO₂NR^{e}R^{f}, —OR^{e}, OC(═O)NR^{e}R^{f}, OC(═O)R^{e} and acyl,

wherein R^{e}, R^{f}, R^{g} and R^{h} are each independently selected from the group consisting of H, C₁-C₁₂alkyl, C₁-C₁₂haloalkyl, C₂-C₁₂alkenyl, C₂-C₁₂alkynyl, C₁-C₁₀heteroalkyl, C₃-C₁₂cycloalkyl, C₃-C₁₂cycloalkenyl, C₁-C₁₂heterocycloalkyl, C₁-C₁₂heterocycloalkenyl, C₆-C₁₈aryl, C₁-C₁₈heteroaryl, and acyl, or any two or more of R^{a}, R^{b}, R^{c} and R^{d}, when taken together with the atoms to which they are attached form a heterocyclic ring system with 3 to 12 ring atoms.

In some embodiments each optional substituent is independently selected from the group consisting of: F, Cl, Br, ═O, ═S, —CN, —NO₂, alkyl, alkenyl, heteroalkyl, haloalkyl, alkynyl, aryl, cycloalkyl, heterocycloalkyl, heteroaryl, hydroxy, hydroxyalkyl, alkoxy, alkylamino, aminoalkyl, acylamino, phenoxy, alkoxyalkyl, benzyloxy, alkylsulfonyl, arylsulfonyl, aminosulfonyl, —C(O)OR^{a}, COOH, SH, and acyl.

In some embodiments each optional substituent is independently selected from the group consisting of: F, Br, Cl, ═O, ═S, —CN methyl, trifluoro-methyl, ethyl, 2,2,2-trifluoroethyl, isopropyl, propyl, 2-ethyl-propyl, 3,3-dimethyl-propyl, butyl, isobutyl, 3,3-dimethyl-butyl, 2-ethyl-butyl, pentyl, 2-methyl-pentyl, pent-4-enyl, hexyl, heptyl, octyl, phenyl, NH₂, —NO₂, phenoxy, hydroxy, methoxy, trifluoro-methoxy, ethoxy, and methylenedioxy.

In some embodiments each optional substituent is independently selected from the group consisting of H, CH₃, CH₂CH₃, CH₂CH₂CH₃, CH(CH₃)₂, (CH₂)₃CH₃, Cl, Br, F, I, OH, NO₂, NH₂, CN, OCH₃, OCH₂CH₂CH₃, CF₃, and OCF₃.

Alternatively, two optional substituents on the same moiety when taken together may be joined to form a fused cyclic substituent attached to the moiety that is optionally substituted. Accordingly the term optionally substituted includes a fused ring such as a cycloalkyl ring, a heterocycloalkyl ring, an aryl ring or a heteroaryl ring.

In addition to compounds of formula (I), the embodiments disclosed are also directed to pharmaceutically acceptable salts, pharmaceutically acceptable N-oxides, pharmaceutically acceptable prodrugs, and pharmaceutically active metabolites of such compounds, and pharmaceutically acceptable salts of such metabolites.

The invention also relates to pharmaceutical compositions including a compound of the invention and a pharmaceutically acceptable carrier, diluent or excipient.

Specific compounds of the invention include the following:

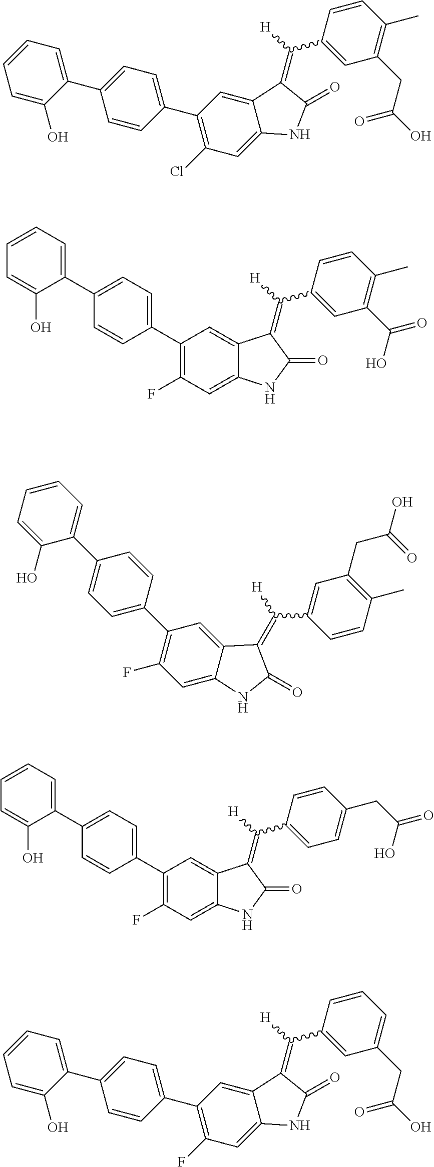

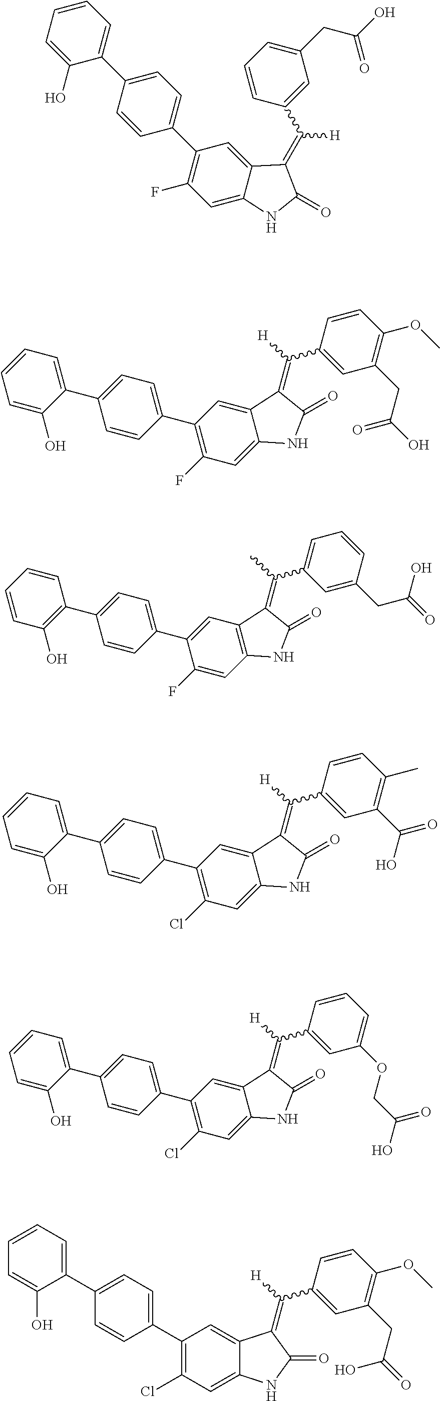

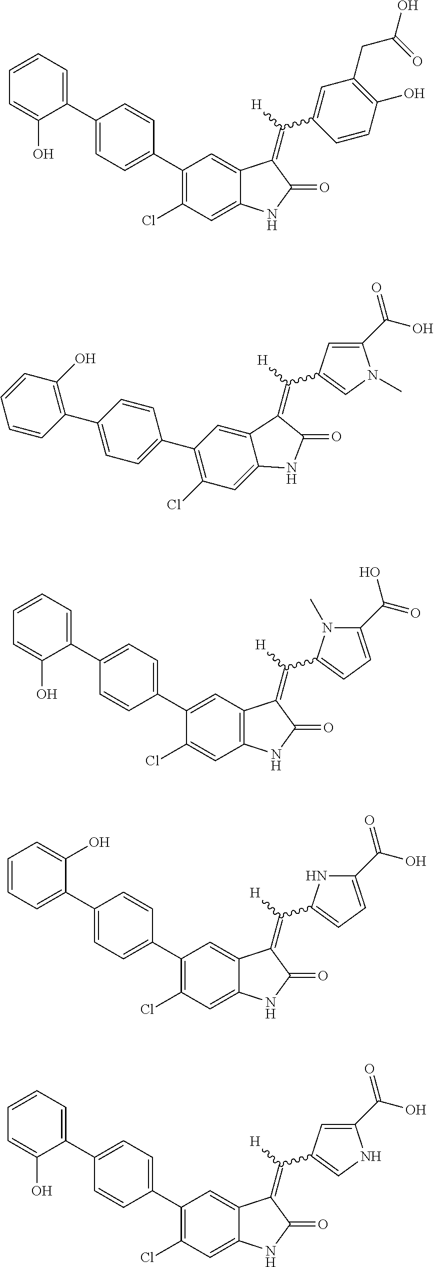

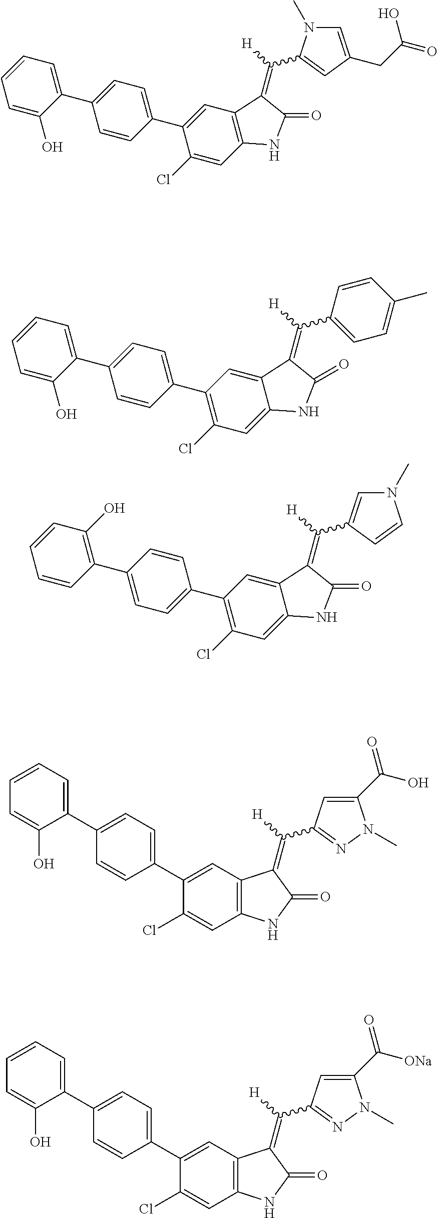

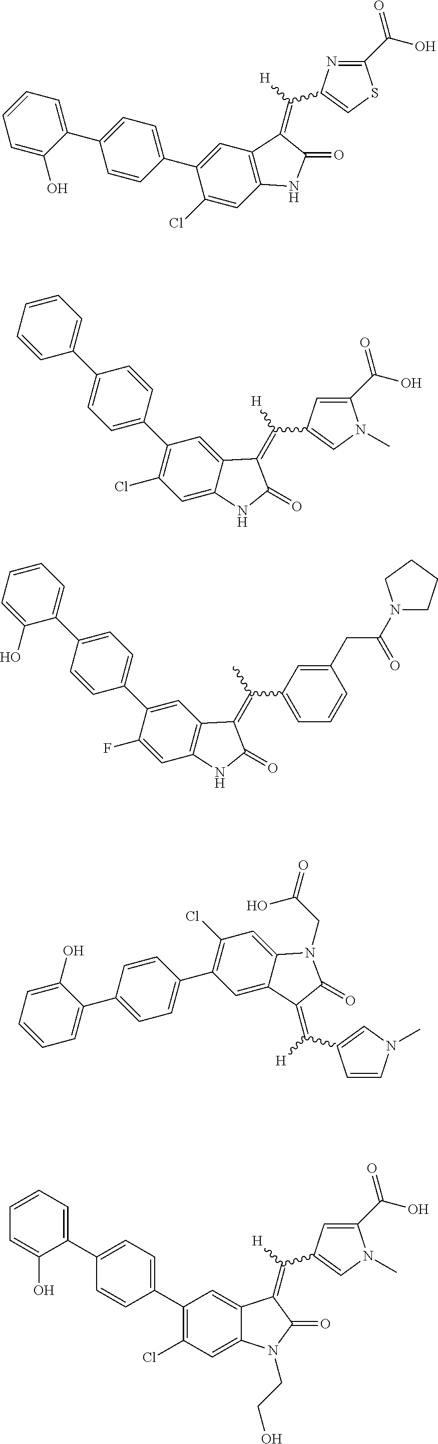

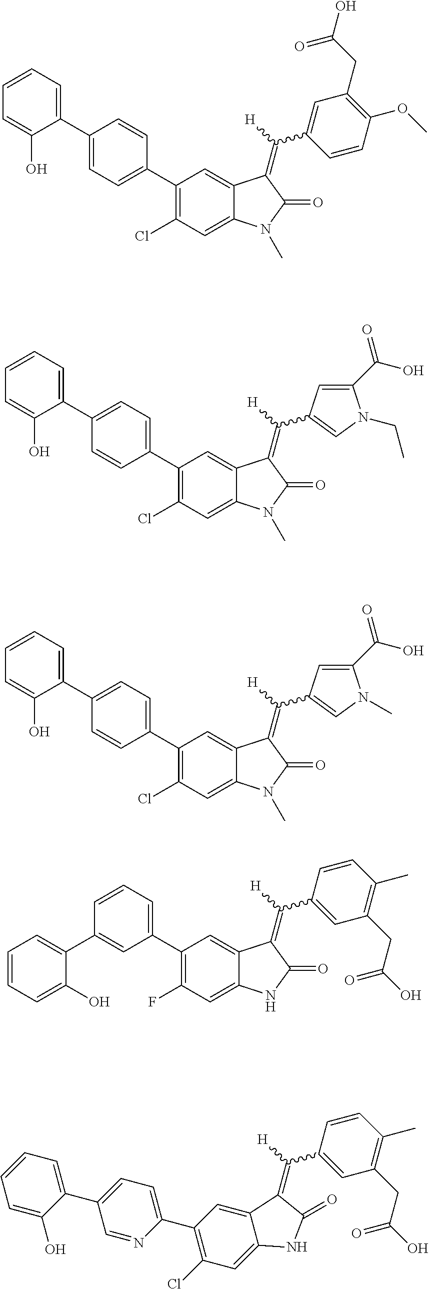

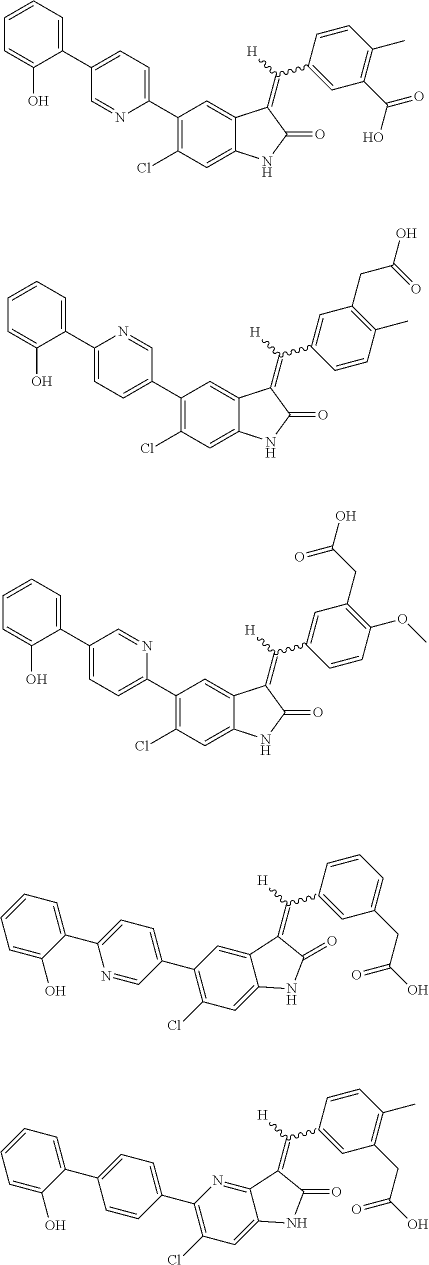

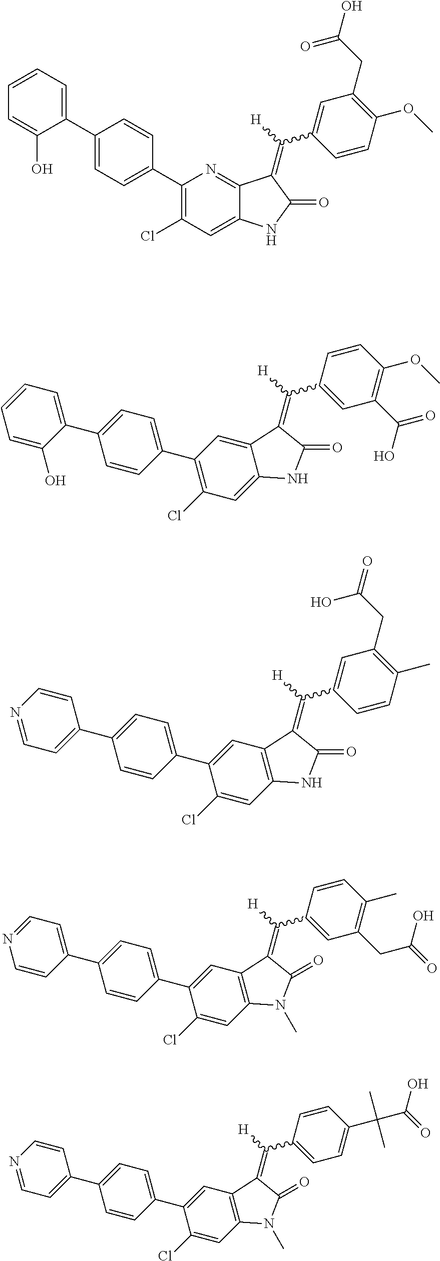

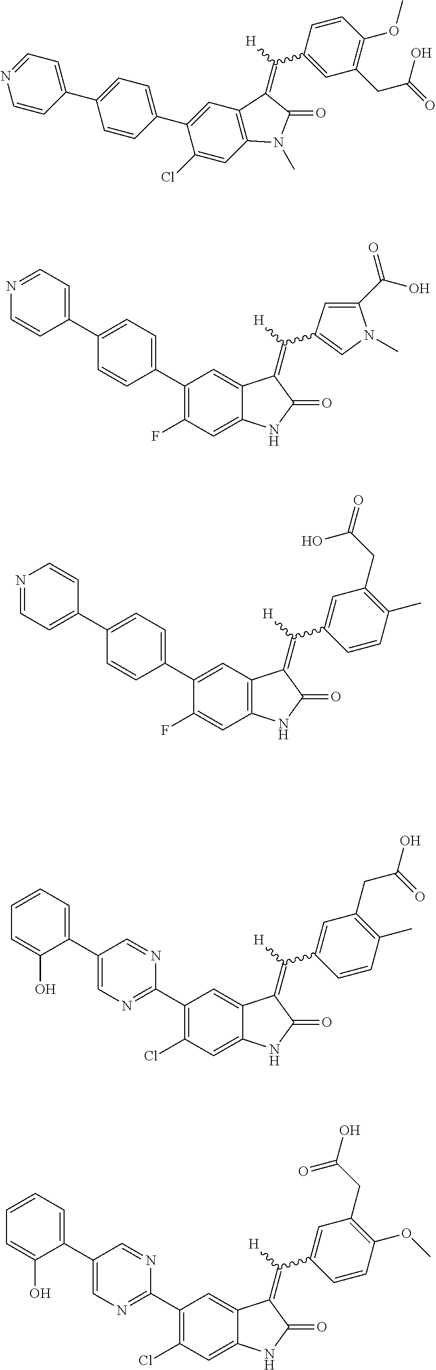

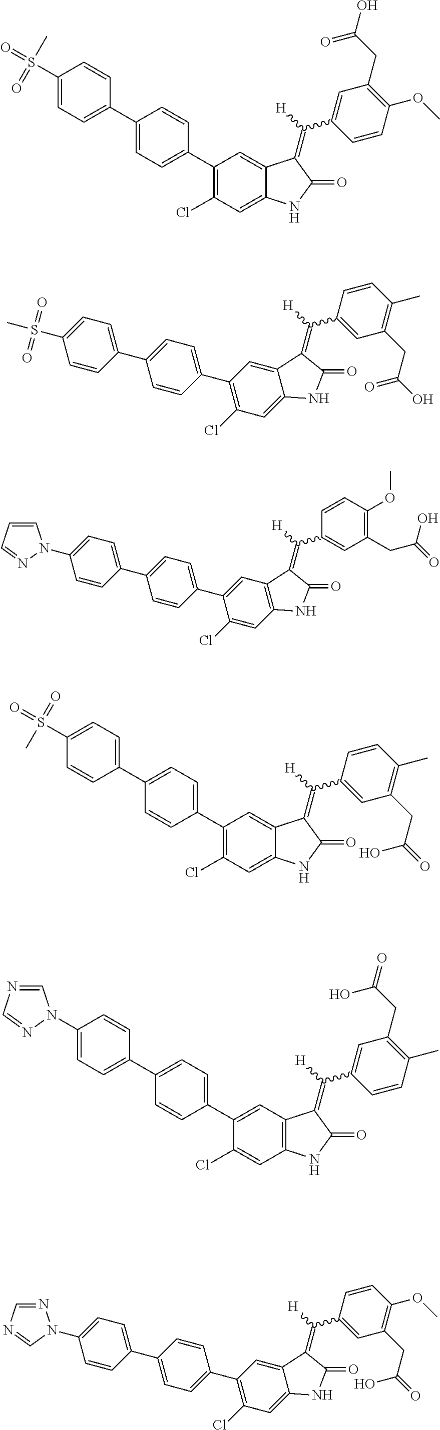

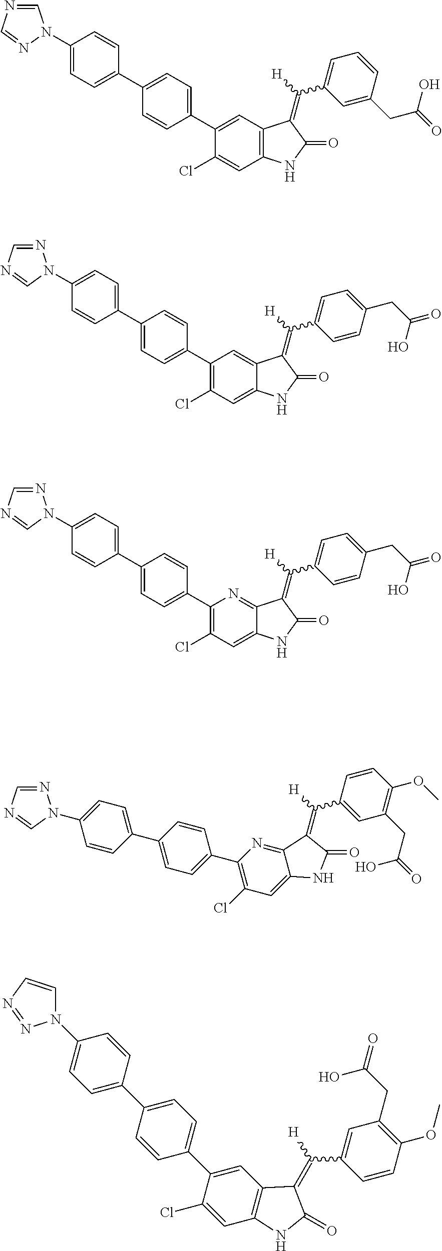

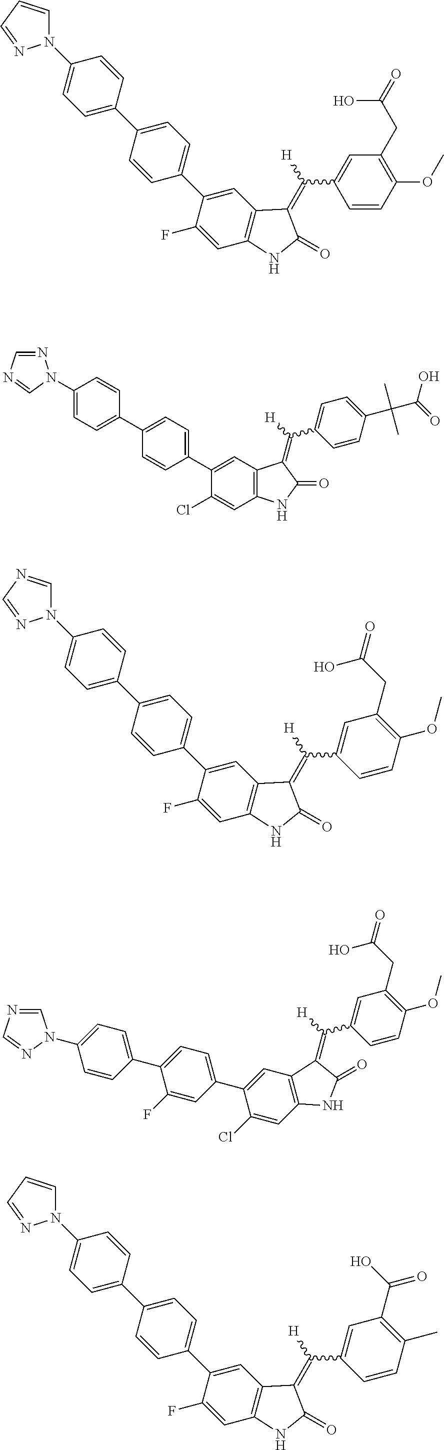

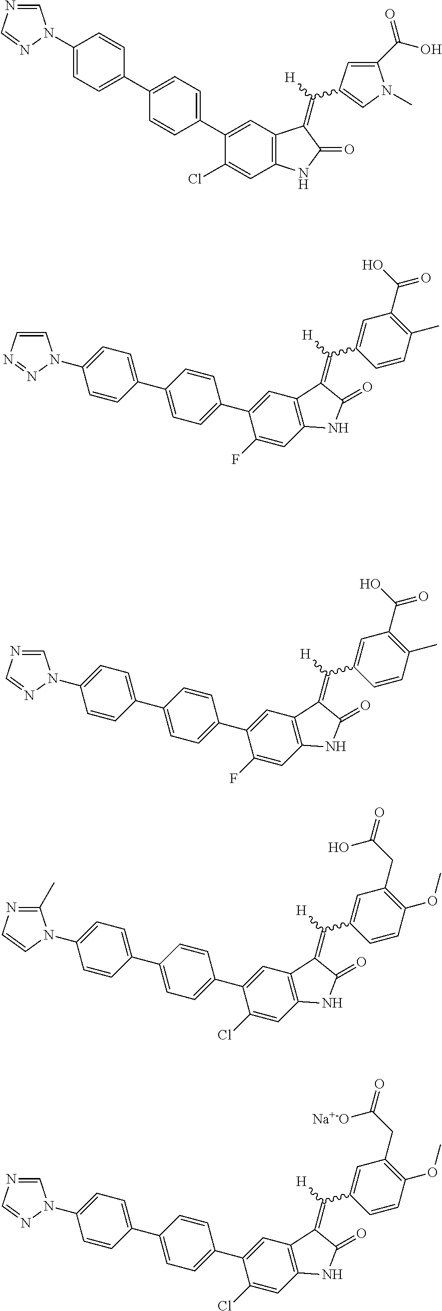

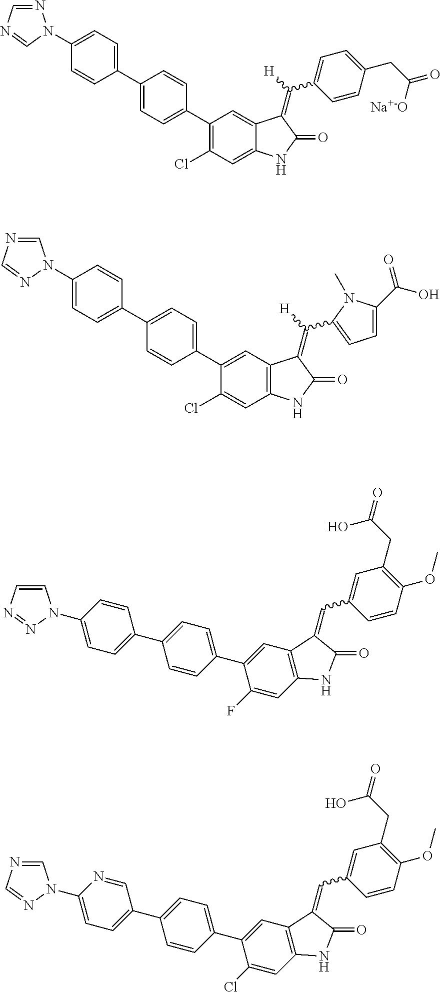

or a pharmaceutically acceptable salt thereof.

The present invention also relates to a pharmaceutical composition comprising a compound according to the invention and a pharmaceutically acceptable diluent, excipient or carrier. In one variation, the composition is a solid formulation adapted for oral administration. In another variation, the composition is a liquid formulation adapted for oral administration. In yet another variation, the composition is a tablet. In still another variation, the composition is a liquid formulation adapted for parenteral administration.

In one aspect of the present invention, there is provided a pharmaceutical composition comprising a compound according to any one of the above compounds, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In another aspect, there is provided a kit comprising any one or more of the above compounds and instructions which comprise one or more forms of information selected from the group consisting of indicating a disease state for which the compound is to be administered, storage information for the compound, dosing information and instructions regarding how to administer the compound. In one aspect, the kit comprises the compound in a multiple dose form.

In yet another aspect there is provided an article of manufacture comprising any one or more of the above compounds and packaging materials. In one variation, the packaging material comprises a container for housing the compound. In one particular variation, the container comprises a label indicating one or more members of the group consisting of a disease state for which the compound is to be administered, storage information, dosing information and/or instructions regarding how to administer the composition. In another variation, the article of manufacture comprises the compound in a multiple dose form.

The compounds have the ability to activate AMPK. The ability activate the receptors may be a result of the compounds acting directly and solely on the receptor to modulate/potentiate biological activity. However, it is understood that the compounds may also act at least partially on other factors associated with the activity of the receptor.

Regulating energy levels is a fundamental process in every living organism and at cellular level. ATP must be maintained at 10-fold excess of ADP concentration to drive essential metabolic processes. AMPK, AMP-activated protein kinase, is emerging as a key player in overall regulation of energy balance. AMPK is activated by ATP depletion and an increase in the AMP/ATP ratio.

AMPK is a heterotrimer complex consisting of a catalytic subunit a and regulatory subunits β and γ. The heterotrimer is assembled from out of α1, α2, β1, β2, β3, γ1, γ2 and γ3 subunits. The α subunit has the serine threonine protein kinase activity in the N-terminus. The β subunit binds both α and γ subunits and has a sequence similarity to N-isoamylase domain that metabolizes α1-6 branch points in α1-4 linked glucans. The γ subunit is reported to bind AMP.

Upon binding AMP, the α subunit is phosphorylated by LKB1 (STK11) at threonine 172 and activated AMPK phosphorylates downstream target proteins. The physiological effect of activation of AMPK appears to be extremely divergent depending on the tissue/organ in which it is activated. Activation of AMPK in the hypothalamus leads to increased food intake by increased expression of neuropeptide Y while inhibition of AMPK (by leptin) reduces food intake. In muscle AMPK activation increases beta-oxidation and energy expenditure.

AMPK is activated in muscle by increase in AMP:ATP ratio and decrease in phosphocreatinine content, both of which occur during exercise. Long term exercise and activation of AMPK in muscle leads to activation of NRF-1 and transcription of genes involved in mitochondrial biogenesis like ALAS-1 and cytochrome C. In muscle AMPK is required for expression of Ca2⁺/Calmodulin activated protein kinase IV (CAMK4). CAMK4 in turn induces expression of PPARGC-1. PPARGC-1 induces expression of and also co-activates NRF1 for the transcription of increased mitochondrial biogenesis which results in enhanced fatty acid oxidation. AMPK phosphorylates ACACB and thereby activates CPT1 and fatty acid oxidation. AMPK enhances transcription of GLUT4 and increases glucose uptake. AMPK enhances expression of glucokinase and activates PK2 by phosphorylation. PFK2 stimulates production of fructose 2,6-bisphosphate which is a physiological activator of 6 phosphofructokinase.

It has been stipulated that AMPK controls gluconeogenesis in multiple ways. AMPK phosphorylates TORC2, a co-activator of CREB that is required for expression of PPARGC1, and sequesters it in the cytoplasm. PPARGC1 is absolutely required for transcription of PCK and G6PC genes. Activated AMPK is also reported to cause degradation of FOXO1 protein and phosphorylation and inactivation of MLLT7, two other transcription factors of PCK gene. Additionally, activated AMPK phosphorylates and inhibits HNF4α and thus shuts off the program of gluconeogenesis in liver. Phosphorylation of HNF4α also results in reduced transcription of ApoB and ApoCIII that results in reduced triglyceride concentration in plasma in vivo.

Cholesterol synthesis is inhibited by AMPK by phosphorylation and inactivation of HMGCoA reductase. AMPK also inhibits lipid synthesis by reduced transcription of SREBP1, phosphorylation and inhibition of WBSCR14, and phosphorylation of ACACA. AMPK also inhibits triacylglycerol formation by phosphorylation of GPAT. AMPK is also known to reduce the mRNA levels of GCK, ALDOB, PKLR and SLC2A2 and thus reduce glucose uptake and metabolism. Glycogen synthase is also phosphorylated and inhibited by activated AMPK. Activated AMPK thus switches off ATP utilizing synthesis pathways.

Simultaneously, activated AMPK phosphorylates and inhibits ACACB (reducing melanoyl CoA production) and also phosphorylates and activates MYCLD (that metabolizes melanoyl CoA) resulting in activation of CPT1. CPT1 (Carnitine Palmitoyl Transferase 1) controls the entry of fatty acids into mitochondria for oxidation.

AMPK controls protein synthesis by targeting protein synthesis initiation and elongation. AMPK phosphorylates and activates eukaryotic elongation factor 2 kinase (eEF2 kinase) that phosphorylates and inactivates elongation factor 2 (eEF2). Simultaneously, AMPK phosphorylates and activates tuberous sclerosis complex 2 (TSC2). TSC2 in turn prevents FRAP1 (mTOR) activation which results in EIF4EBP1 remaining unphosphorylated and active and thereby inhibiting translation initiation. Activated TSC2 also results in inhibition of RPS6KB1 and thus protein synthesis is inhibited. Sustained activation of AMPK in liver results in enhancement of apoptosis by increase in activity of caspase 3 (CASP3) and c-JUN.

Furthermore, in adipose tissue, activation of AMPK results in an increase in beta-oxidation of fatty acids and reduces the triacylglycerol accumulation that enhances insulin sensitivity of the organ.

Activated AMPK causes a decrease in the transcription of PPARγ and C/EBPα mRNA levels that directly affects lipogenesis and decreases accumulation of triacylglycerol in adipocytes. At the same time AMPK also phosphorylates eIF2α, AGPAT and DGAT and reduces triacylglycerol synthesis. As in liver, AMPK phosphorylates and inactivates ACACA (reducing melanoyl CoA production) and phosphorylates and activates MLYCD (thereby increasing melanoyl CoA metabolism) to effectively reduce melanoyl concentration that is essential to activate CPT1 and mitochondrial fatty acid oxidation. In keeping with its role to increase beta-oxidation, AMPK phosphorylates and inactivates hormone sensitive lipase, LIPE, and inhibits lipolysis and reduces free fatty acids in circulation. AMPK also increases GLUT4 mRNA and increases glucose uptake. AMPK reduces inflammatory process in adipocytes by inhibiting, post-transcriptionally, inducible nitric oxide synthase (NOS2A) protein levels and by decreasing secretion of IL6, CCL3, CCL4 and TNFRS1B.

The activation of AMPK may be carried out in any of a number of well known ways in the art. For example if activation in vitro is desired an appropriate amount of the compound may be added to a solution containing the AMPK. In circumstances where it is desired to activate the AMPK in a mammal, the activation of the AMPK typically involves administering the compound to a mammal capable of producing the AMPK protein.

Accordingly the compounds may find a multiple number of applications in which their ability to activate AMPK of the type mentioned above can be utilised.

In a further aspect the present invention provides a method of prevention or treatment of a condition associated with associated with 5′ AMP-activated protein kinase activity in a mammal, the method comprising administering an effective amount of a compound of the invention.

In yet an even further aspect the invention provides the use of a compound of the invention in the preparation of a medicament for the prevention or treatment of a condition associated with 5′ AMP-activated protein kinase activity in a mammal.

In yet an even further aspect the invention provides a compound of the invention for use in the treatment of a condition associated with 5′ AMP-activated protein kinase activity in a mammal.

Accordingly compounds of the invention would be expected to have useful therapeutic properties especially in relation to metabolic conditions such as diabetes, obesity, hyperglycemia, glucose intolerance, insulin resistance, hyperinsulemia, hypercholesteremia, hypertension, hyperlipoproteinemia, hyperlipidemia, hypertriglyceridemia, dyslipedemia, metabolic syndrome X, atherosclerosis, diabetic neuropathy, diabetic retinopathy, and hypoglycemia.

Compounds of the invention may also be useful in the treatment of cognitive disorders, osteoporosis, inflammatory disorders, cardiovascular disease, kidney disease, ketoacidosis, thrombotic disorders, nephropathy, sexual dysfunction, dermatopathy, dyspepsia, cancer and edema. As such there is significant interest in the development of compounds with this mode of action.

In some embodiments the condition is diabetes. In some embodiments the condition is type II diabetes.

Administration of compounds within Formula (I) to humans can be by any of the accepted modes for enteral administration such as oral or rectal, or by parenteral administration such as subcutaneous, intramuscular, intravenous and intradermal routes. Injection can be bolus or via constant or intermittent infusion. The active compound is typically included in a pharmaceutically acceptable carrier or diluent and in an amount sufficient to deliver to the patient a therapeutically effective dose. In various embodiments the activator compound may be selectively toxic or more toxic to rapidly proliferating cells, e.g. cancerous tumours, than to normal cells.

In using the compounds of the invention they can be administered in any form or mode which makes the compound bioavailable. One skilled in the art of preparing formulations can readily select the proper form and mode of administration depending upon the particular characteristics of the compound selected, the condition to be treated, the stage of the condition to be treated and other relevant circumstances. We refer the reader to Remingtons Pharmaceutical Sciences, 19^{th} edition, Mack Publishing Co. (1995) for further information.

The compounds of the present invention can be administered alone or in the form of a pharmaceutical composition in combination with a pharmaceutically acceptable carrier, diluent or excipient. The compounds of the invention, while effective themselves, are typically formulated and administered in the form of their pharmaceutically acceptable salts as these forms are typically more stable, more easily crystallised and have increased solubility.

The compounds are, however, typically used in the form of pharmaceutical compositions which are formulated depending on the desired mode of administration. As such in some embodiments the present invention provides a pharmaceutical composition including a compound of Formula (I) and a pharmaceutically acceptable carrier, diluent or excipient. The compositions are prepared in manners well known in the art.

The invention in other embodiments provides a pharmaceutical pack or kit comprising one or more containers filled with one or more of the ingredients of the pharmaceutical compositions of the invention. In such a pack or kit can be found a container having a unit dosage of the agent(s). The kits can include a composition comprising an effective agent either as concentrates (including lyophilized compositions), which can be diluted further prior to use or they can be provided at the concentration of use, where the vials may include one or more dosages. Conveniently, in the kits, single dosages can be provided in sterile vials so that the physician can employ the vials directly, where the vials will have the desired amount and concentration of agent(s). Associated with such container(s) can be various written materials such as instructions for use, or a notice in the form prescribed by a governmental agency regulating the manufacture, use or sale of pharmaceuticals or biological products, which notice reflects approval by the agency of manufacture, use or sale for human administration.

The compounds of the invention may be used or administered in combination with one or more additional drug(s) for the treatment of the disorder/diseases mentioned. The components can be administered in the same formulation or in separate formulations. If administered in separate formulations the compounds of the invention may be administered sequentially or simultaneously with the other drug(s).

In addition to being able to be administered in combination with one or more additional drugs, the compounds of the invention may be used in a combination therapy. When this is done the compounds are typically administered in combination with each other. Thus one or more of the compounds of the invention may be administered either simultaneously (as a combined preparation) or sequentially in order to achieve a desired effect. This is especially desirable where the therapeutic profile of each compound is different such that the combined effect of the two drugs provides an improved therapeutic result.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminium metahydroxide, bentonite, agar-agar, and tragacanth, and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, patches, sprays, ointments and inhalants. The active compound is mixed under sterile conditions with a pharmaceutically acceptable carrier and any needed preservatives, buffers, or propellants which may be required.

The amount of compound administered will preferably treat and reduce or alleviate the condition. A therapeutically effective amount can be readily determined by an attending diagnostician by the use of conventional techniques and by observing results obtained under analogous circumstances. In determining the therapeutically effective amount a number of factors are to be considered including but not limited to, the species of animal, its size, age and general health, the specific condition involved, the severity of the condition, the response of the patient to treatment, the particular compound administered, the mode of administration, the bioavailability of the preparation administered, the dose regime selected, the use of other medications and other relevant circumstances.

A preferred dosage will be a range from about 0.01 to 300 mg per kilogram of body weight per day. A more preferred dosage will be in the range from 0.1 to 100 mg per kilogram of body weight per day, more preferably from 0.2 to 80 mg per kilogram of body weight per day, even more preferably 0.2 to 50 mg per kilogram of body weight per day. A suitable dose can be administered in multiple sub-doses per day.

### Synthesis of Compounds of the Invention

The agents of the various embodiments may be prepared using the reaction routes and synthesis schemes as described below, employing the techniques available in the art using starting materials that are readily available.

The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T. W. Greene's Protective Groups in Organic Synthesis, 3^{rd} Edition, John Wiley & Sons, 1991. Alternatively, other reactions disclosed herein or known in the art will be recognized as having applicability for preparing other compounds of the various embodiments.

Reagents useful for synthesizing compounds may be obtained or prepared according to techniques known in the art.

The symbols, abbreviations and conventions in the processes, schemes, and examples are consistent with those used in the contemporary scientific literature. Specifically but not meant as limiting, the following abbreviations may be used in the examples and throughout the specification.
g (grams)
L (liters)
Hz (Hertz)
mol (moles)
RT (room temperature)
min (minutes)
MeOH (methanol)
CHCl₃ (chloroform)
DCM (dichloromethane)
DMSO (dimethylsulfoxide)
EtOAc (ethyl acetate)
mg (milligrams)
mL (milliliters)
psi (pounds per square inch)
mM (millimolar)
MHz (megahertz)
h (hours)
TLC (thin layer chromatography)
EtOH (ethanol)
CDCl₃ (deuterated chloroform)
HCl (hydrochloric acid)
DMF (N, N-dimethylformamide)
THF (tetrahydrofuran)
K₂CO₃ (potassium carbonate)
Na₂SO₄ (sodium sulfate)
RM (Reaction Mixture)

Unless otherwise indicated, all temperatures are expressed in ° C. (degree centigrade). All reactions conducted at room temperature unless otherwise mentioned.

All the solvents and reagents used are commercially available and purchased from Sigma Aldrich, Fluka, Acros, Spectrochem, Alfa Aesar, Avra, Qualigens, Merck, Rankem and Leonid Chemicals.

¹H NMR spectra were recorded on a Bruker AV 300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on single quadruple 6120 LCMS from Agilent technologies, using either atmospheric chemical ionization (APCI) or Electrospray ionization (ESI) or in the combination of these two sources.

All samples were run on SHIMADZU system with an LC-20 AD pump, SPD-M20A diode array detector, SIL-20A auto sampler.

### Synthetic Schemes

Scheme for making the certain compounds of the invention is shown in scheme 1 below.

### Intermediate 1: 5-Bromo-6-fluoro-1,3-dihydro-2H-indol-2-one

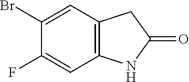 [SMILES: O=C1Cc2cc(Br)c(F)cc2N1]

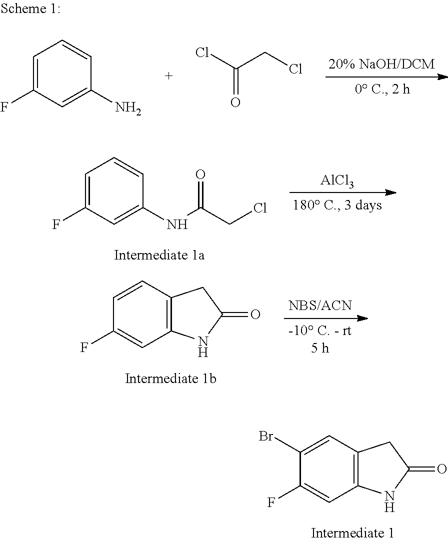

### Intermediate 1a: 2-Chloro-N-(3-fluorophenyl)acetamide

To a solution of 3-Fluoroaniline (100.0 g, 900.0 mmol) in DCM (1 L) at 0° C., 20% NaOH solution (600 mL) was added followed by addition of chloroacetylchloride (121.9 g, 1080.0 mmol) and stirred at 0° C. for 2 h. The reaction mixture was diluted with water and extracted with ethyl acetate. The organic layer was washed with 1N HCl, followed by a wash with water and then dried over anhydrous Na₂SO₄. The crude product was purified by crystallization with n-hexane to yield the title compound (140.0 g, 93.0%) as an off white solid. ¹H NMR: (DMSO-d₆, 300 MHz) δ 10.51 (s, 1H), 7.56-7.60 (d, 1H), 7.30-7.42 (m, 2H), 6.93-6.96 (d, 1H), 4.27 (s, 2H).

### Intermediate 1 b: 6-Fluoro-1,3-dihydro-2H-indol-2-one

To a 1 L RBF charged with 2-chloro-N-(3-fluorophenyl)acetamide (70.0 g, 373.1 mmol), AlCl₃ (248.7 g, 1859.9 mmol) was added and heated at 180° C. for 3 days. The reaction mixture was diluted with water and filtered through celite. The product was extracted with ethyl acetate, washed with water, and dried over anhydrous Na₂SO₄. The solvent was removed under vacuo, the obtained crude product was purified by column chromatography to yield the title compound (37.0 g, 65.61%) as an off white solid. ¹H NMR: (DMSO-d₆, 300 MHz) δ 10.51 (s, 1H), 7.18-7.22 (m, 1H), 6.69-6.76 (m, 1H), 6.60-6.63 (m, 1H), 3.45 (s, 2H).

### Intermediate 1: 5-Bromo-6-fluoro-1,3-dihydro-2H-indol-2-one

To a 6-fluoro-1,3-dihydro-2H-indol-2-one (3.0 g, 19.8 mmol) in acetonitrile (100 mL) at 0° C., NBS (4.24 g, 23.8 mmol) was added portion wise under nitrogen atmosphere and was maintained at −10° C. for 1 h. It was allowed to stir at room temperature for 5 h. The reaction mixture was diluted with water and extracted with ethyl acetate, washed with water and dried over anhydrous Na₂SO₄. The solvent was removed under vacuo to yield the crude product, which was triturated with n-hexane to yield the title compound (3.5 g, 76.76%) as a pale yellow solid.

### Intermediate 2: (2′-Hydroxybiphenyl-4-yl)boronic acid

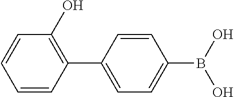 [SMILES: OB(O)c1ccc(-c2ccccc2O)cc1]

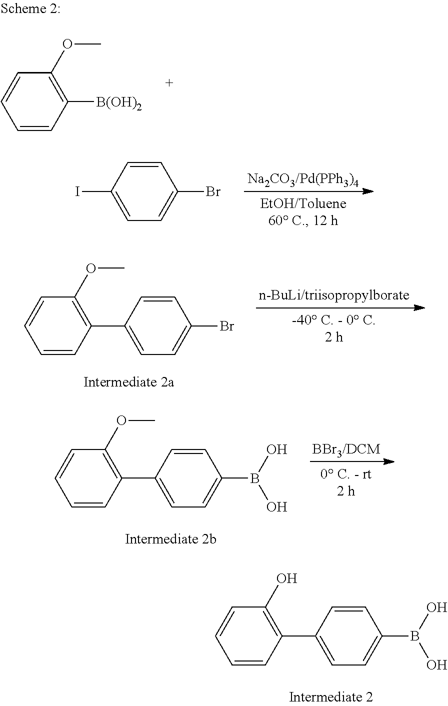 [SMILES: COc1ccccc1B(O)O ; Brc1ccc(I)cc1 ; COc1ccccc1-c1ccc(Br)cc1 ; COc1ccccc1-c1ccc(B(O)O)cc1 ; OB(O)c1ccc(-c2ccccc2O)cc1]

### Intermediate 2a: 4′-Bromo-2-methoxybiphenyl

A 500 mL sealed tube was charged with (2-methoxyphenyl)boronic acid (12.0 g, 42.4 mmol), 1-bromo-4-iodo benzene (6.44 g, 42.4 mmol) and mixture of toluene and ethanol (300 mL). To the above stirred solution 2M Na₂CO₃ solution (30 mL) was added. After degasified with nitrogen for 30 min Pd(PPh₃)₄ (0.49 g, 0.424 mmol) was added and heated at 60° C. for 12 h. The reaction mixture diluted water and extracted with ethyl acetate, the combined organic layer was washed with water and brine solution. The solvent was removed under vacuo. The product was purified by column chromatography to yield the title compound (7.0 g, 62.7%) as a yellow crystalline solid. ¹H NMR: (CDCl₃, 300 MHz) δ 7.44-7.47 (d, 2H), 7.29-7.34 (m, 2H), 7.18-7.26 (m, 2H), 6.90-6.98 (m, 2H), 3.74 (s, 3H).

### Intermediate 2b: (2′-Methoxybiphenyl-4-yl)boronic acid

To a stirred solution of 4′-Bromo-2-methoxybiphenyl (7.0 g, 26.6 mmol) in dry THF (70 mL) at −40° C., n-BuLi (12.8 mL) was added and maintained at −40° C. for 1 h. To the above solution triispropylborate (10.01 g, 53.2 mmol) was added drop wise at −40° C. and temperature was raised to 0° C. over a period for 2 h. The reaction mixture was quenched with saturated AlCl₃ solution and NaOH solution. It was washed with ether and acidified with 1N HCl solution, extracted with ethyl acetate, with water and brine solution. The solvent was dried over anhydrous Na₂SO₄, removed under vacuo and washed with n-hexane to yield the title product (4.8 g, 80.0%) as a brown solid. LCMS: (M−H)⁺=227.0.

### Intermediate 2: (2′-Hydroxybiphenyl-4-yl)boronic acid

To a stirred solution of (2′-methoxybiphenyl-4-yl)boronic acid (4.1 g, 18.0 mmol) in DCM (40 mL) at 0° C., BBr₃ (2.6 mL) was added drop wise and maintained at same temperature for 30 min and stirred at room temperature for 2 h. The reaction mixture was cooled to 0° C. This was followed by addition of methanol and the reaction mixture was stirred at room temperature 15 min. The solvent was distilled off under reduced pressure; the residue was dissolved in ethyl acetate, washed with water and brine solution. The solvent was dried over anhydrous Na₂SO₄ and concentrated under reduced pressure to yield the title product (2.7 g, 70.10%) as a brown solid. LCMS: (M−H)⁺=213.1.

### Intermediate A: 6-Fluoro-5-(2′-hydroxybiphenyl-4-yl)-1,3-dihydro-2H-indol-2-one

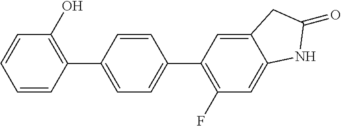 [SMILES: O=C1Cc2cc(-c3ccc(-c4ccccc4O)cc3)c(F)cc2N1]

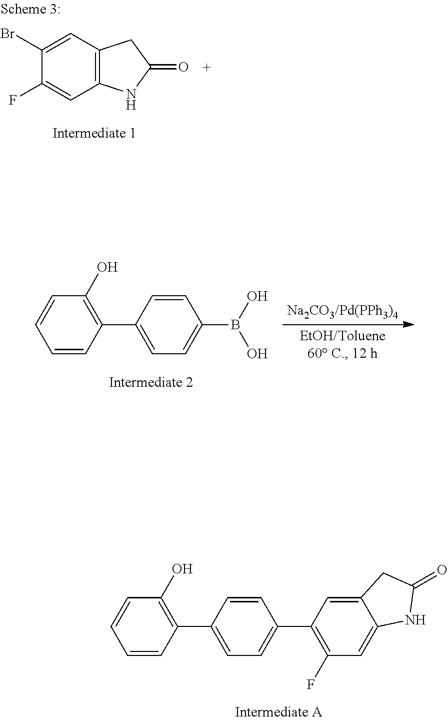 [SMILES: O=C1Cc2cc(Br)c(F)cc2N1 ; OB(O)c1ccc(-c2ccccc2O)cc1 ; O=C1Cc2cc(-c3ccc(-c4ccccc4O)cc3)c(F)cc2N1]

To a solution of Intermediate 1 (0.92 g, 4.0 mmol) and Intermediate 2 (1.0 g, 4.7 mmol) potassium phosphate tribasic (2.1 g, 9.9 mmol) in 1,4-dioxane (7 mL) and water (3 mL) was added under argon atmosphere. This was followed by addition of Pd(PPh₃)₄(0) (0.23 g 0.2 mmol) and heated at 90° C. overnight. The reaction mixture was partitioned between water and ethyl acetate. The combined organic layer was dried over anhydrous Na₂SO₄ and concentrated under vacuo. The obtained crude product was purified by triruration with n-hexane and diethyl ether to yield the title product (0.4 g, 31.3%) as beige solid. LCMS: (M−H)⁺=318.1.

### Intermediate 3: 5-Bromo-6-chloro-1,3-dihydro-2H-indol-2-one

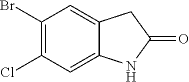 [SMILES: O=C1Cc2cc(Br)c(Cl)cc2N1]

To a solution of 6-chlorooxiindole (5.0 g, 29.8 mmol) in acetonitrile (50 mL) at −10° C., NBS (5.8 g, 32.8 mmol) was added. The reaction mixture was stirred at room temperature for 3 h. The reaction was monitored by LC-MS. The reaction mixture was evaporated to dryness and extracted with ethyl acetate. The ethyl acetate layer was washed with water and dried over anhydrous Na₂SO₄ and concentrated. This was further purified by trituration with n-hexane to yield title compound (7.0 g, 95.8%) as a brown solid. LCMS: (M−H)⁺=244.9; ¹H NMR: (DMSO-d₆, 300 MHz) δ 10.61 (s, 1H), 7.56 (s, 1H), 6.98 (s, 1H), 3.51 (s, 2H).

### Intermediate B: 6-Chloro-5-(2′-hydroxybiphenyl-4-yl)-1,3-dihydro-2H-indol-2-one

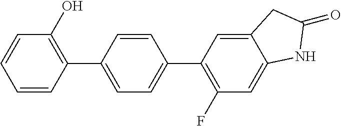 [SMILES: O=C1Cc2cc(-c3ccc(-c4ccccc4O)cc3)c(F)cc2N1]

The Intermediate B was synthesized by using intermediate 2 (2.6 g, 1.0 eq) and Intermediate 3 (3.0 g, 1.0 eq) by following the similar procedure as described in intermediate A. It was obtained as a pale white solid 2.6 g, 6.74%).

### Intermediate 4: Methyl 5-formyl-2-methylbenzoate

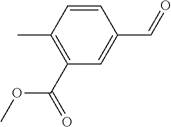

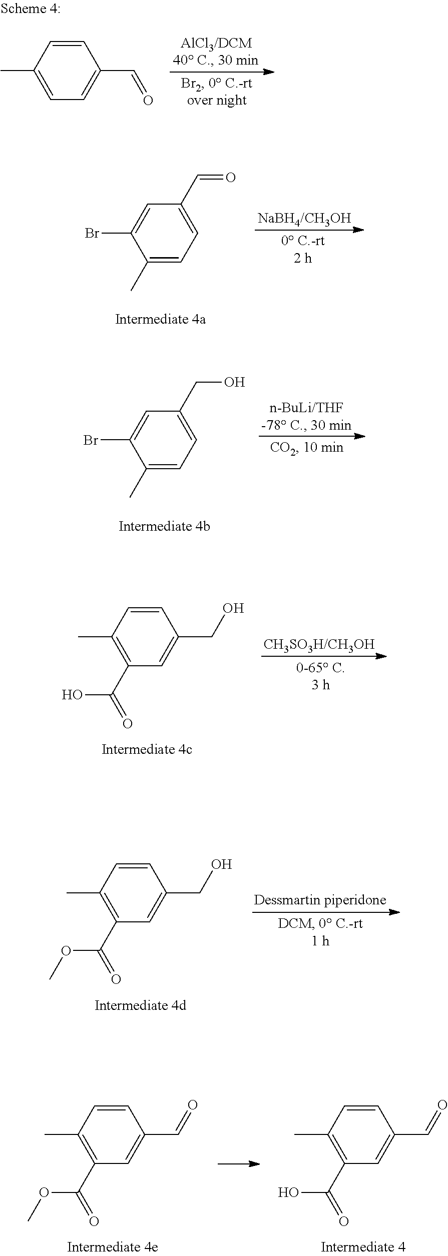

### Intermediate 4a: 3-Bromo-4-methylbenzaldehyde

To a stirred solution of 4-methylbenzaldehyde (20.0 g, 166.5 mmol) in DCM (150 mL) at 0° C. AlCl₃ (26.5 g, 198.7 mmol) was added portion wise. It was heated at 40° C. for 30 min, then Br₂ (31.9 g, 199.6 mmol) in DCM (50 mL) was added drop wise at 0° C. and stirred at room temperature over night. The reaction mixture was diluted with ice water and extracted with DCM. The organic layer was washed with water and brine solution dried over anhydrous Na₂SO₄ and concentrated under vacuo. The crude product was purified by column chromatography to yield the title compound (20 g, 60.36%) as yellow oil.

### Intermediate 4b: (3-Bromo-4-methylphenyl) methanol

To a stirred solution of 3-bromo-4-methylbenzaldehyde (10.0 g, 50.2 mmol) in dry methanol (60 mL) at 0° C., NaBH₄ (2.3 g, 36.0 mmol) was added portion wise and stirred at room temperature for 2 h. The reaction mixture was quenched with ice water, extracted with ethyl acetate; the organic layer was washed with water and brine solution, dried over anhydrous Na₂SO₄ and concentrated. The product was purified by column chromatography to yield title compound (5.0 g, 49.60%) as yellow oil.

### Intermediate 4c: 5-(Hydroxymethyl)-2-methylbenzoic acid

To a stirred solution of (3-bromo-4-methylphenyl) methanol (5.5 g, 27.4 mmol) in THF (50 mL) at −78° C., n-BuLi [(18.6 g, 2.5 eq (2.5M soln) was added and the solution was stirred at same temperature for 30 min. Then, CO₂ gas was purged (generated from dry ice) for about 10 min. The reaction mixture was quenched with ammonium chloride and acidified with 1N HCl, extracted with ethyl acetate, the organic layer was washed with water, brine solution. Then, it was dried over anhydrous Na₂SO₄ and concentrated under reduced pressure and washed with n-hexane to obtain the title compound (4.0 g, 87.85%) as an off white solid. LCMS: 165.0 (M−H)⁺; ¹H NMR: (CDCl₃, 300 MHz) δ 12.67 (s, 1H), 7.79 (s, 1H), 7.36-7.38 (d, 1H), 7.23-7.25 (d, 1H), 5.23 (s, 1H), 4.49 (s, 2H), 2.50 (s, 3H).

### Intermediate 4d: Methyl 5-(hydroxymethyl)-2-methylbenzoate

To a stirred solution of 5-(hydroxymethyl)-2-methylbenzoic acid (4.0 g, 24.1 mmol) in methanol (40 mL) at 0° C., methanesulfonic acid (2.3 g, 23.9 mmol) was added and the solution was heated at 65° C. for 3 h. After cooling, the solvent was evaporated and the residue was extracted with ethyl acetate. The combined organic layer was washed with water, brine, dried over anhydrous Na₂SO₄ and concentrated under reduced pressure. The product was purified by chromatography to yield the title product (3.5 g, 80.50%) as yellow oil. ¹H NMR: (DMSO-d₆, 300 MHz) δ 7.90 (s, 1H), 7.39-7.42 (d, 1H), 7.23-7.26 (d, 1H), 4.69 (s, 2H), 3.89 (s, 3H), 2.59 (s, 3H).

### Intermediate 4e: Methyl 5-formyl-2-methylbenzoate

To a stirred solution of methyl 5-(hydroxymethyl)-2-methylbenzoate (3.5 g, 19.4 mmol) in DCM (15 mL) at 0° C., dessmartin periodinane (12.3 g, 33.0 mmol) was added portion wise and stirred at same temperature for 10 min and continued to be stirred at room temperature for 1 h. The reaction mixture was diluted with water, extracted with ethyl acetate; the organic layer was washed with water, brine solution dried over anhydrous Na₂SO₄, filtered and concentrated. It was purified by combiflash to yield the title product (2.65 g, 76.80%) as a white solid. LCMS: 179.1 (M+H)⁺; ¹H NMR: (CDCl₃, 300 MHz) δ 10.01 (s, 1H), 8.42 (s, 1H), 7.91-7.93 (d, 1H), 7.42-7.44 (d, 1H), 3.94 (s, 3H), 2.70 (s, 3H).

### Intermediate 4: 5-formyl-2-methylbenzoic acid

Intermediate 4e was treated with 1N NaOH in EtOH/THF/H2O (7:2:1) to afford title compound.

### Intermediate 5: (5-Formyl-2-methylphenyl) acetic acid

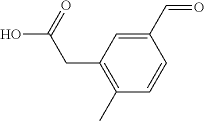

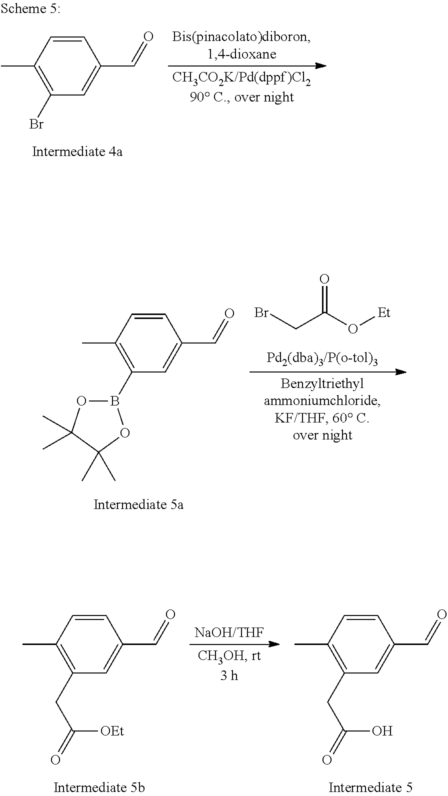

### Intermediate 5a: 4-Methyl-3-(4,4,5,5-tetramethyl-1,3,2-dioxaborolan-2-yl)benzaldehyde

To a stirred solution of intermediate 4a (3.0 g, 15.1 mmol) in 1,4 dioxone (30 mL), bis(pinocolate)diboran (4.5 g, 17.7 mmol) and potassium acetate (2.98 g, 30.4 mmol) was added. The solution was then degasified with nitrogen for 20 min. Then, Pd(dppf)Cl₂ (0.62 g, 0.75 mmol) was added. It was heated at 90° C. over night. The reaction mixture was diluted with water and extracted with ethyl acetate; the organic layer was washed with water, brine solution and concentrated. The product was purified by combiflash to yield the title product (3.4 g, 91.39%) as a yellow semi solid. LCMS: (M+H)⁺=247.2; ¹H NMR: (CDCl₃, 300 MHz) δ 9.99 (s, 1H), 8.26 (s, 1H), 7.82-7.85 (dd, 1H), 7.26-7.33 (m, 1H), 2.62 (s, 3H), 1.37 (s, 12H).

### Intermediate 5b: Ethyl (5-formyl-2-methylphenyl) acetate

A 500 mL sealed tube was charged with of 4-methyl-3-(4,4,5,5-tetramethyl-1,3,2-dioxaborolan-2-yl)benzaldehyde (3.4 g, 13.8 mmol), tris(dibenzylideneacetone)-dipalladium (0) (0.38 g, 0.41 mmol), tris(o-tolyl)phosphino (0.38 g, 1.24 mmol), benzyltriethylammoniumchloride (0.38 g, 1.38 mmol), potassium fluoride 2.4 g, 41.3 mmol) and dry THF (34 mL). Then, it was degasified with nitrogen for about 20 minfollowed by addition of ethyl bromoacetate (3.46 g, 20.7 mmol). Then, reaction mixture was heated at 60° C. over night. The reaction mixture was diluted with water and extracted with ethyl acetate, the organic layer was washed with water, brine and concentrated. The product was purified by combiflash to yield the title product (1.1 g, 38.41%) as a yellow liquid. LCMS: (M+H)⁺=207.1; ¹H NMR: (CDCl₃, 300 MHz) δ 9.89 (s, 1H), 7.63-7.65 (d, 2H), 7.27-7.30 (d, 1H), 4.07-4.1 (q, 2H), 3.64 (s, 2H), 2.33 (s, 3H), 1.17-1.217 (t, 3H).

### Intermediate 5: (5-Formyl-2-methylphenyl)acetic acid

To a stirred solution of compound ethyl (5-formyl-2-methylphenyl) acetate (3.5 g, 16.9 mmol) in THF (6 mL) and methanol (6 mL), sodium hydroxide solution (2.03 g, 50.8 mmol) was added and stirred at room temperature for 3 h. The solvent was removed under reduced pressure; the obtained residue was washed with ethyl acetate. The aqueous layer was acidified with 1N HCl and extracted with ethyl acetate. It was then washed with water and brine solution, dried over anhydrous Na₂SO₄ and concentrated under vacuo. The obtained crude product was purified by column chromatography to yield title compound (2.55 g, 82.78%) as an off white solid. LCMS: (M+H)⁺=179.1

### Intermediate 6: 4-Formylphenylacetic acid

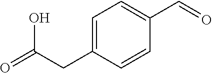 [SMILES: O=Cc1ccc(CC(=O)O)cc1]

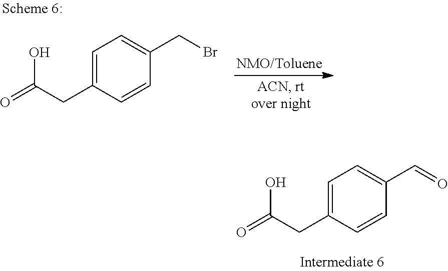 [SMILES: O=C(O)Cc1ccc(CBr)cc1 ; O=Cc1ccc(CC(=O)O)cc1]

To a stirred solution of [4-(bromomethyl) phenyl]acetic acid (1.0 g, 4.4 mmol) in acetonitrile (15 mL) and toluene (7.5 mL), N-Methylmorpholine N-oxide (1.5 g, 12.81 mmol) was added and stirred at room temperature for over night. The reaction mixture was concentrated. The residue was diluted with water and extracted with ethyl acetate; the organic layer was washed with water, brine solution, dried over anhydrous Na₂SO₄ and concentrated. The product was purified by combiflash to yield the title product (0.3 g, 41.5%) as a white solid.

### Intermediate 7: 3-Formylphenylacetic acid

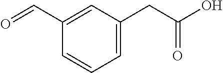 [SMILES: O=Cc1cccc(CC(=O)O)c1]

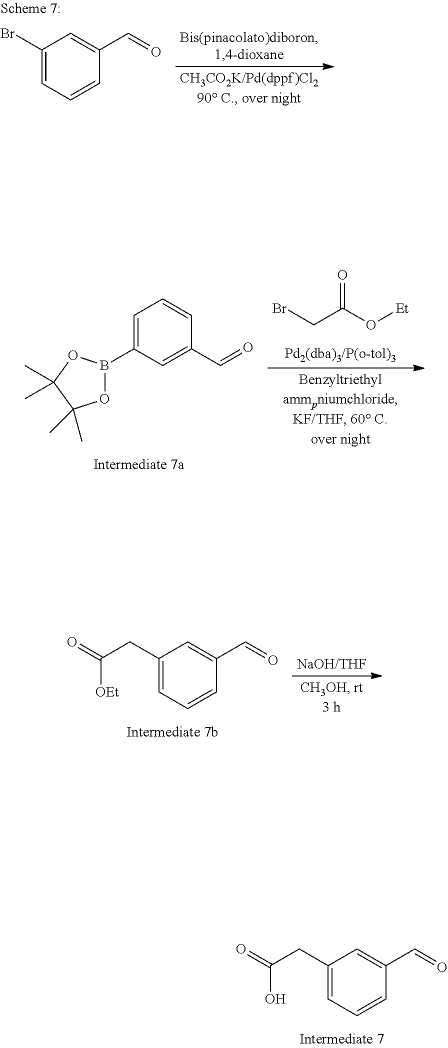 [SMILES: O=Cc1cccc(Br)c1 ; CC1(C)OB(c2cccc(C=O)c2)OC1(C)C ; CCOC(=O)CBr ; CCOC(=O)Cc1cccc(C=O)c1 ; O=Cc1cccc(CC(=O)O)c1]

### Intermediate 7a: 3-(4,4,5,5-Tetramethyl-1,3,2-dioxaborolan-2-yl)benzaldehyde

A 500 mL sealed tube was charged with 3-bromobenzaldehyde (9.25 g, 50.0 mmol), potassium acetate (9.8 g, 1.3 mmol), bis(pinacolato)diboran (15.3 g, 60.3 mmol) in 1,4-dioxane (50 mL) and was degasified with nitrogen for 15 min. Then, Pd(dppf)Cl₂ (0.82 g, 31.7 mmol) was added to it and the reaction mixture thus obtained was heated at 90° C. over night. The reaction mixture was filtered through celite and the filtrate was partitioned between water and ethyl acetate. The combined organic layer was dried over anhydrous Na₂SO₄ and evaporated. The product was purified by column chromatography to yield the title product (11.5 g, 99.102%) as a colorless liquid. LCMS: (M+H)⁺=233.2; ¹H NMR: (DMSO-d₆, 300 MHz) δ 10.06 (s, 1H), 8.21 (s, 1H), 8.01-8.04 (d, 1H), 7.96-7.99 (d, 1H), 7.61-7.66 (t, 1H), 1.33 (s, 12H).

### Intermediate 7b: Ethyl (3-formylphenyl) acetate

A 500 mL sealed tube was charged with 3-(4,4,5,5-tetramethyl-1,3,2-dioxaborolan-2-yl)benzaldehyde (11.5 g, 49.6 mmol), potassium fluoride (8.7 g, 149.7 mmol), tris(o-tolyl)phosphino (1.37 g, 4.5 mmol), benzyltriethyl ammonium chloride (1.14 g, 5.0 mmol) and Pd₂(dba)₃ (1.37 g, 1.50 mmol). It was then degasified for 10 min under nitrogen atmosphere after which ethylbromoacetate (12.4 g, 74.3 mmol) in THF (50 mL) was added to it before stirring the tube at 60° C. over night. The reaction mixture was diluted with water and extracted with ethyl acetate, dried over anhydrous Na₂SO₄ and concentrated. The product was purified by column chromatography to yield the title product (5.2 g, 54.54%) as a pale yellow liquid. LCMS: (M+H)⁺=192.1; ¹H NMR: (DMSO-d₆, 300 MHz) δ 10.01 (s, 1H), 7.81-7.84 (m, 2H), 7.54-7.65 (m, 2H), 4.06-4.13 (q, 2H), 3.81 (s, 2H), 1.17-1.22 (t, 3H).

### Intermediate 7: 3-Formylphenylacetic acid

To a solution of ethyl (3-formylphenyl)acetate (0.3 g, 1.3 mmol) in methanol (3 mL), LiOH (0.19 g, 5.0 eq) in water (1 mL) was added and stirred at room temperature for 2 h. The reaction mixture was concentrated, the obtained residue was diluted with water and extracted with ethyl acetate; the organic layer was washed with water, brine solution, over anhydrous Na₂SO₄ and concentrated. It was purified by column chromatography to yield the title product (0.07 g, 26.6%) as a white solid. LCMS: (M−H)⁺=163.0

### Intermediate 8: Ethyl (5-formyl-2-methoxyphenyl)acetate

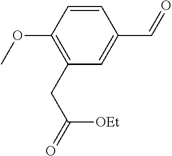 [SMILES: CCOC(=O)Cc1cc(C=O)ccc1OC]

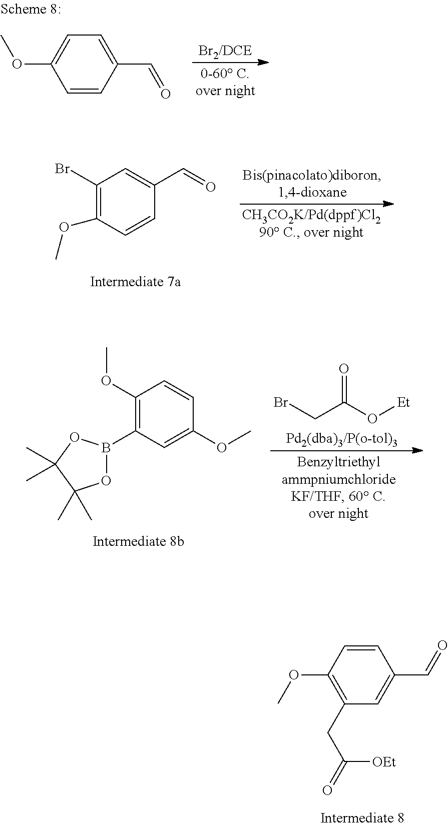

### Intermediate 8a: 3-Bromo-4-methoxybenzaldehyde

To a stirred solution of 4-methoxybenzaldehyde (30.0 g, 220.4 mmol) was taken in DCE (300 mL), at 0° C. was added bromine (38.7 g, 242.2 mmol) drop wise. It was heated at 60° C. for over night. The reaction mixture was quenched with ice water, and then extracted with ethyl acetate. The organic layer was washed with sodium thiosulphate, water and brine solution, over anhydrous Na₂SO₄ and concentrated. The product was purified by column chromatography to yield the title product (20.0 g, 42.1%) as an off white solid.

### Intermediate 8b: 4-Methoxy-3-(4,4,5,5-tetramethyl-1,3,2-dioxaborolan-2-yl)benzaldehyde

To a stirred solution of intermediate 8a (9.0 g, 41.9 mmol), 1,4 dioxane (90 mL), bis(pinocalato)diboran (12.7 g, 50.0 mmol) and potassium acetate (8.28 g, 2.0 eq) was added and was degasified using nitrogen. Then, Pd(dppf)Cl₂ (1.7 g, 0.05 eq) was added and was heated at 90° C. over night. The reaction mixture was diluted with water and extracted with ethyl acetate; the organic layer was washed with water, brine solution, dried over anhydrous Na₂SO₄ and concentrated. The product was purified by combi-flash to yield the title product (9.0 g, 73.76%) as an off white solid. LCMS: (M+H)+=263.2; ¹H NMR: (CDCl₃, 300 MHz) δ 9.83 (s, 1H), 8.13-8.14 (d, 1H), 7.87-7.91 (m, 1H), 1 6.89-6.92 (d, 1H), 3.85 (s, 3H), 1.30 (s, 12H).

### Intermediate 8: Ethyl (5-formyl-2-methoxyphenyl)acetate

To a 500 mL sealed tube charged with intermediate 8b (9.0 g, 34.3 mmol) in dry THF (90 mL), Pd₂(dba)₃(0) (0.94 g, 0.03 eq), tris(o-tolyl)phosphino (0.94 g, 0.09 eq), benzyltriethylammoniumchloride (0.78 g, 0.1 eq) and potassium fluoride (5.98 g, 3.0 eq) was added and the tube was then degasified with nitrogen. This was followed by addition of ethyl bromoacetate (8.6 g, 51.5 mmol) and was again degasified for 10 min. Then, it was heated at 60° C. over night. The reaction mixture was diluted with water and extracted with ethyl acetate, the organic layer was washed with water, brine solution, dried over anhydrous Na₂SO₄ and concentrated. The product was purified by combi-flash to yield the title product (3.5 g, 45.77%) as a yellow liquid, LCMS: (M+H)+=223.1; ¹H NMR: (CDCl₃, 300 MHz) δ 9.80 (s, 1H), 7.73-7.76 (m, 2H), 6.90-6.93 (d, 1H), 4.06-4.13 (q, 2H), 3.84 (s, 3H), 3.59 (s, 2H), 1.16-1.21 (t, 3H).

### Intermediate 9: Ethyl (3-acetylphenyl)acetate

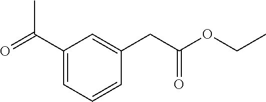

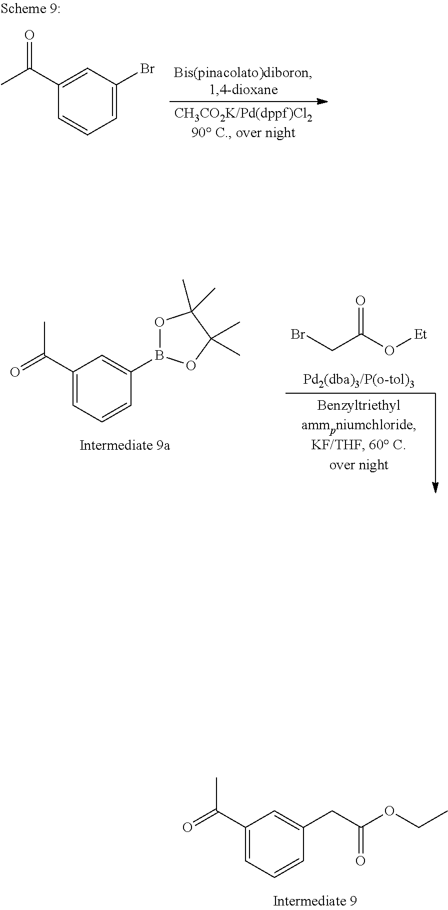

### Intermediate 9a: 1-[3-(4,4,5,5-Tetramethyl-1,3,2-dioxaborolan-2-yl)phenyl] ethanone

To a solution of 1-(3-bromophenyl)ethanone (1.0 g, 5.0 mmol) in 1,4-dioxane (5 mL), bis(pinacolato)diboron (1.53 g, 6.0 mmol) and potassium acetate (0.32 g, 3.3 mmol) was added. The reaction mixture was degasified with nitrogen, then Pd(dppf)Cl₂ (0.026 g, 0.02 eq) was added and the reaction mixture was maintained at 90° C. over night. After cooling, the reaction mixture was diluted with water and extracted with ethyl acetate anhydrous Na₂SO₄ and concentrated. It was then purified by column chromatography to yield the title product (1.14 g, 92.6%) as a pale yellow oil. ¹H NMR: (CDCl₃, 300 MHz) δ 8.29 (s, 1H), 7.91-8.01 (m, 2H), 7.38-7.43 (t, 1H), 2.57 (s, 3H), 1.29 (s, 12H).

### Intermediate 9: (3-Acetylphenyl)acetate

A 100 mL sealed tube was charged with 1-[3-(4,4,5,5-tetramethyl-1,3,2-dioxaborolan-2-yl)phenyl] ethanone (0.7 g, 2.8 mmol), benzyltriethylammonium chloride (0.065 g, 0.29 mmol), potassium fluoride (0.49 g, 8.5 mmol) and tris(o-tolyl)phosphino (0.077 g, 0.25 mmol) and THF (5 mL). To the above solution Pd(dppf)Cl₂ (0.078 g, 0.1 mmol) with THF (5 mL) was added. The reaction mixture was degasified with nitrogen, before addition of ethyl bromoacetate (0.87 g, 1.5 eq) and the reaction mixture is then heated at 60° C. over night. After cooling, the reaction mass was partitioned between water and ethyl acetate. The combined organic layer was dried over anhydrous Na₂SO₄ and concentrated. The product was purified by combiflash to yield the title product (0.3 g, 51.28%) as pale yellow oil. LCMS: (M+H)⁺=207.1

### Intermediate 10: (3-Formylphenoxy)acetic acid

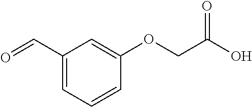 [SMILES: O=Cc1cccc(OCC(=O)O)c1]

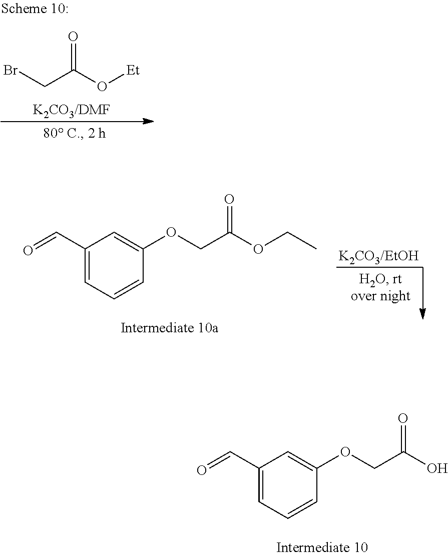

### Intermediate 10a: Ethyl (3-formylphenoxy) acetate

To a solution of 3-hydroxybenzaldehyde (5.0 g, 40.9 mmol) in DMF (30 mL), ethyl bromoacetate (8.75 g, 52.4 mmol) and potassium carbonate (7.2 g, 52.1 mmol) was added and heated at 80° C. for 2 h. After cooling, the reaction mixture was quenched with ice cold water and extracted with diethyl ether. The combined organic layer was washed with water and dried over anhydrous Na₂SO₄ and concentrated. The product was purified by combiflash to yield the title product (7.0 g, 84.13%).

### Intermediate 10: (3-Formylphenoxy)acetic acid

To a solution of ethyl (3-formylphenoxy) acetate (0.5 g, 2.4 mmol) in ethanol (3 mL) and water (3 mL) potassium carbonate (1.0 g, 7.2 mmol) was added and stirred at room temperature over night. The reaction mixture was partitioned between water and ether. The aqueous layer was acidified with 1N HCl and the extracted with ethyl acetate. The combined organic layer was dried over anhydrous Na₂SO₄ and concentrated under vacuo to yield the title product (0.35 g, 81.0%) as an off-white solid.

### Intermediate 11: 4-Formyl-1-methyl-1H-pyrrole-2-carboxylic acid

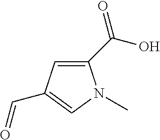

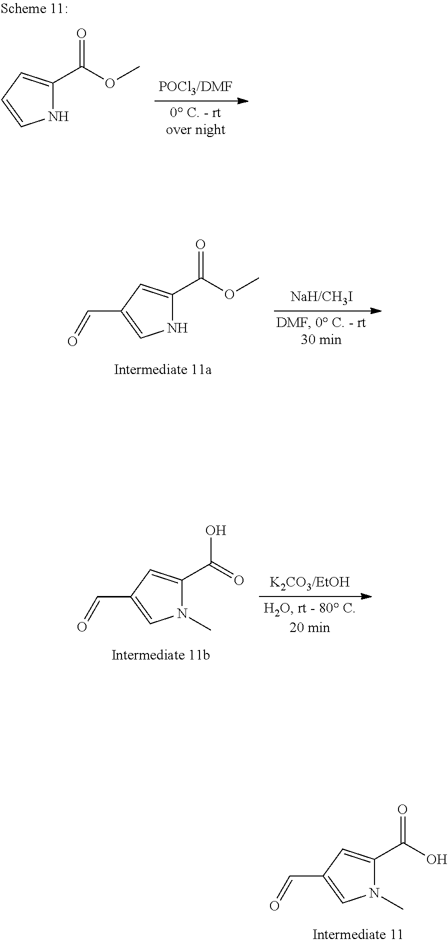

### Intermediate 11a: Methyl 4-formyl-1H-pyrrole-2-carboxylate

To the stirred solution of methyl 1H-pyrrole-2-carboxylate (0.9 g, 7.19 mmol) in DMF (4.2 g, 57.52 mmol) at 0° C., POCl₃ (5.5 g, 35.96 mmol) was added. The reaction mixture was stirred at 10° C. for 30 min and at room temperature over night. The reaction mixture was quenched with sodium hydroxide solution and extracted using ethyl acetate dried over anhydrous Na₂SO₄. The crude product was purified by column chromatography to yield the title compound polar spot (0.53 g, 48.18%) as a pale yellow solid. LCMS: (M−H)⁺=152.1; ¹H NMR: (DMSO-d₆, 300 MHz) δ 12.73 (s, 1H), 7.76 (s, 1H), 7.82-7.83 (d, 1H), 7.14 (s, 2H), 3.81 (s, 3H).

### Intermediate 11 b: Methyl 4-formyl-1-methyl-1H-pyrrole-2-carboxylate

To a solution of methyl 4-formyl-1H-pyrrole-2-carboxylate (0.15 g, 098 mmol) in DMF (4 mL) at 0° C. NaOH (0.047 g (95%), 1.96 mmol) was added and stirred at 0° C. for 15 min. Then methyl iodide (0.208 g, 1.47 mmol) was added and stirred at room temperature for 30 min. The reaction mixture was quenched with water and extracted using ethyl acetate dried over anhydrous Na₂SO₄ and concentrated to yield the title compound (0.16 g, 95.72%) as a pale brown solid. LCMS: (M+H)⁺=168.1; ¹H NMR: (DMSO-d₆, 300 MHz) δ 9.71 (s, 1H), 7.91 (s, 2H), 7.22-7.23 (d, 2H), 3.92 (s, 3H), 3.78 (s, 3H).

### Intermediate 11: 4-Formyl-1-methyl-1H-pyrrole-2-carboxylic acid

To a solution of methyl 4-formyl-1-methyl-1H-pyrrole-2-carboxylate (0.16 g, 0.96 mmol) in ethanol (5 mL), potassium carbonate (0.27 g, 1.92 mmol) was added in water (1 mL) and stirred at room temperature for 15 min and heated at 50° C. for 20 min. The reaction mixture was concentrated and acidified with 1N HCl and extracted using ethyl acetate, dried over anhydrous Na₂SO₄ and concentrated to yield the title compound (0.14 g, 95.51%) as a pale yellow solid. LCMS: (M−H)⁺=152.1; ¹H NMR: (DMSO-d₆, 300 MHz) δ 12.76 (s, 1H), 9.69 (s, 1H), 7.86-7.87 (d, 1H), 7.16-7.17 (d, 1H), 3.91 (s, 3H).

### Intermediate 12: 5-Formyl-1-methyl-1H-pyrrole-2-carboxylic acid

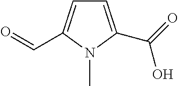

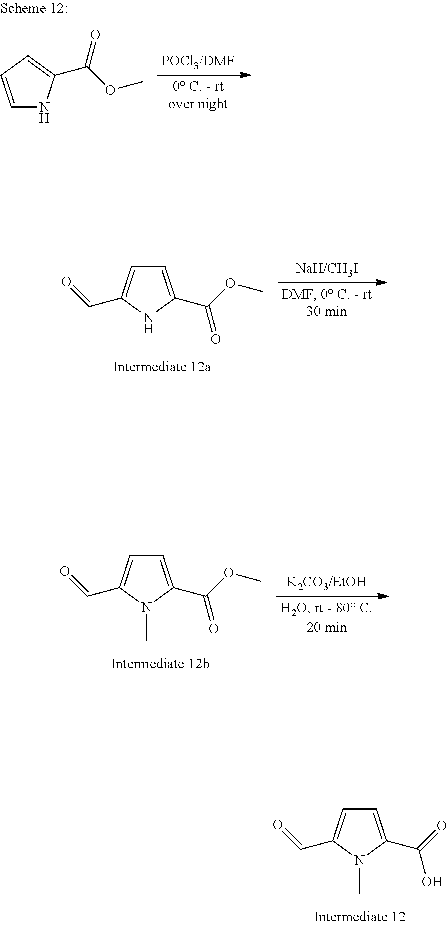

### Intermediate 12a: Methyl 5-formyl-1H-pyrrole-2-carboxylate

It has been synthesized from methyl 1H-pyrrole-2-carboxylate by following the same procedure described as in intermediate 11a, to yield the title compound (0.5 g, 45.45%) as a pale yellow solid. LCMS: (M−H)⁺=152.1.

### Intermediate 12b: Methyl 5-formyl-1-methyl-1H-pyrrole-2-carboxylate

To the solution of methyl 5-formyl-1H-pyrrole-2-carboxylate (0.15 g, 0.98 mmol) in DMF (4 mL) at 0° C. NaH (0.047 g (95%), 1.96 mmol) was added and stirred at same temperature for 15 min. To the above solution methyl iodide (0.208 g, 1.47 mmol) was added and stirred at room temperature for 30 min. The reaction mixture was quenched with water and extracted with ethyl acetate, dried over anhydrous Na₂SO₄ and concentrated to yield the title compound (0.16 g, 95.72%) as a pale yellow solid. LCMS: (M+H)⁺=168.1; ¹H NMR: (DMSO-d₆, 300 MHz) δ 9.76 (s, 1H), 7.05-7.06 (d, 1H), 6.92-6.94 (d, 1H), 4.17 (s, 3H), 3.82 (s, 3H).

### Intermediate 12: 5-Formyl-1-methyl-1H-pyrrole-2-carboxylic acid

To the solution of methyl 5-formyl-1-methyl-1H-pyrrole-2-carboxylate (0.16 g, 0.96 mmol) in ethanol (5 mL) potassium carbonate (0.265 g, 1.92 mmol) in water (1 mL) was added and stirred at room temperature for 15 min and at 50° C. for 20 min. The reaction mixture was concentrated and acidified with 1N HCl and extracted using ethyl acetate, dried over anhydrous Na₂SO₄ and concentrated. The crude product was purified by column chromatography to yield the title compound as a colorless solid (0.108 g, 73.67%). LCMS: (M−H)⁺=152.0; ¹H NMR: (DMSO-d₆, 300 MHz) δ 13.17 (s, 1H), 9.74 (s, 1H), 7.02-7.04 (d, 1H), 6.87-6.89 (d, 1H), 4.17 (s, 3H).

### Intermediate 13: 5-Formyl-1H-pyrrole-2-carboxylic acid

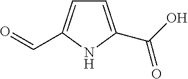

To a solution of intermediate 12a (0.1 g, 0.7 mmol) in mixture of solvents methanol (1 mL) and THF (1 mL), NaOH (0.13 g, 3.25 mmol) was added and stirred at 0° C. for 3 h. The solvent was removed under reduced pressure; salt was washed with diethyl ether to remove impurities. It was acidified with 1N HCl, extracted with ethyl acetate. The combined organic layer was dried and evaporated to yield title product (0.080 g, 88.0%) as beige solid. ¹H NMR: (DMSO-d₆, 300 MHz) δ 13.08 (s, 1H), 12.89 (s, 1H), 9.69 (s, 1H), 6.94 (s 1H), 6.84 (s 1H).

### Intermediate 14: 1-Methyl-1H-pyrrole-3-carbaldehyde

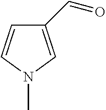

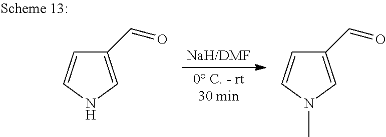

To a suspension of NaH (0.036 g, 1.5 mmol) in DMF (1 mL) at 0° C., a solution of 1H-pyrrole-3-carbaldehyde (0.1 g, 1.05 mmol) in DMF (1 mL) was added and stirred at room temperature for 30 min. Then, methyl iodide (0.46 g, 3.2 mmol) was added to the above solution at 0° C. and stirred at room temperature for 1 h. The reaction mixture was quenched with ice and extracted with ethyl acetate; organic layer was washed with water and brine, dried and concentrated. The crude product was purified by combi-flash to yield the title compound (0.04 g, 35.0%) as a colourless oil. ¹H NMR: (CDCl₃, 300 MHz) δ 9.66 (s, 1H), 7.18-7.21 (m, 1H), 6.56-6.57 (s, 2H), 3.65 (s 3H).

### Intermediate 15: Ethyl 3-formyl-1-methyl-1H-pyrazole-5-carboxylate

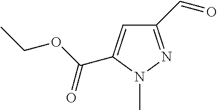

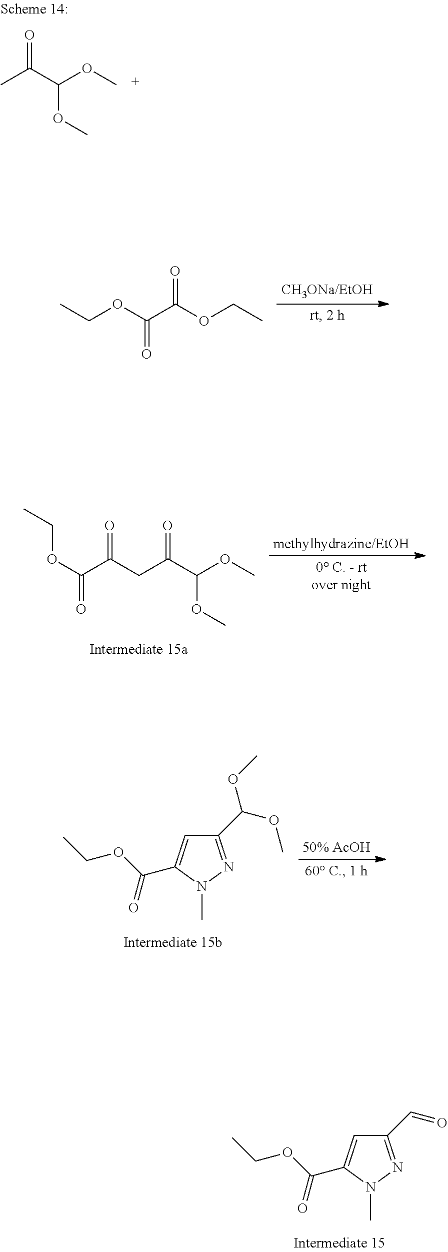

### Intermediate 15a: Ethyl 5,5-dimethoxy-2,4-dioxopentanoate

To a solution of 1,1-dimethoxypropan-2-one (1.0 g, 8.0 mmol) and diethyl ethanedioate (1.48 g, 10.0 mmol) in dry ethanol, freshly prepared sodium ethoxide solution in ethanol was added at room temperature and stirred for 2 h. The reaction mixture was concentrated and the obtained title product (4.0 g, 81.0%), which was used as such for the next step.

### Intermediate 15b: Ethyl 3-(dimethoxymethyl)-1-methyl-1H-pyrazole-5-carboxylate

To a solution of ethyl 5,5-dimethoxy-2,4-dioxopentanoate (4.0 g, 18.3 mmol) in ethanol (3 mL) at 0° C. methyl hydrazine (0.8 g, 17.4 mmol) was added and the resulting mixture was stirred at room temperature over night. The reaction mixture was diluted with diethyl ether, filtered and subsequently washed with ether and concentrated. The crude product was purified by combi-flash to yield the title compound (1.0 g, 23.0%) as pale brown oil.

### Intermediate 15: Ethyl 3-formyl-1-methyl-1H-pyrazole-5-carboxylate

A solution of ethyl 3-(dimethoxymethyl)-1-methyl-1H-pyrazole-5-carboxylate (0.1 g, 0.44 mmol) in 50% aqueous acetic acid (2 mL) were heated at 60° C. for 1 h. The reaction mixture was concentrated under vacuo and the obtained crude product was purified by chromatography to yield the title compound (0.04 g, 50.13%) as a white solid. LCMS: (M+H)⁺=183.1.

### Intermediate 16: 4-Formyl-1,3-thiazole-2-carboxylic acid

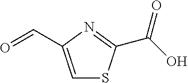

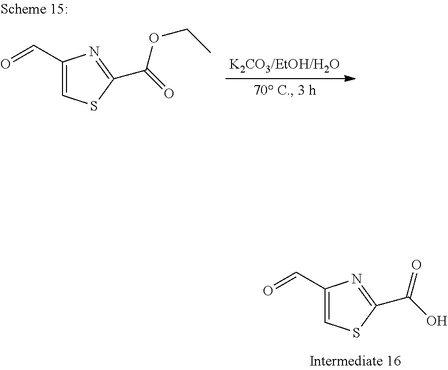

To a solution of ethyl 4-formyl-1,3-thiazole-2-carboxylate (0.04 g, 0.2 mmol) in ethanol (2 mL), aqueous potassium carbonate solution in water (1 mL) was added and the resulting mixture was heated at 70° C. for 3 h. The reaction mixture was concentrated and neutralized with 1 N HCl and then extracted with ethyl acetate. The organic layer was washed with water and brine, dried and concentrated to yield the title compound (0.018 g, 57.0%) as a pale brown solid. ¹H NMR: (CDCl₃, 300 MHz) δ 14.38-14.49 (br, 1H), 10.01 (s, 1H), 8.96 (s, 1H).

### Intermediate 17: Ethyl (5-formyl-1-methyl-1H-pyrrol-3-yl)acetate

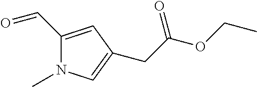

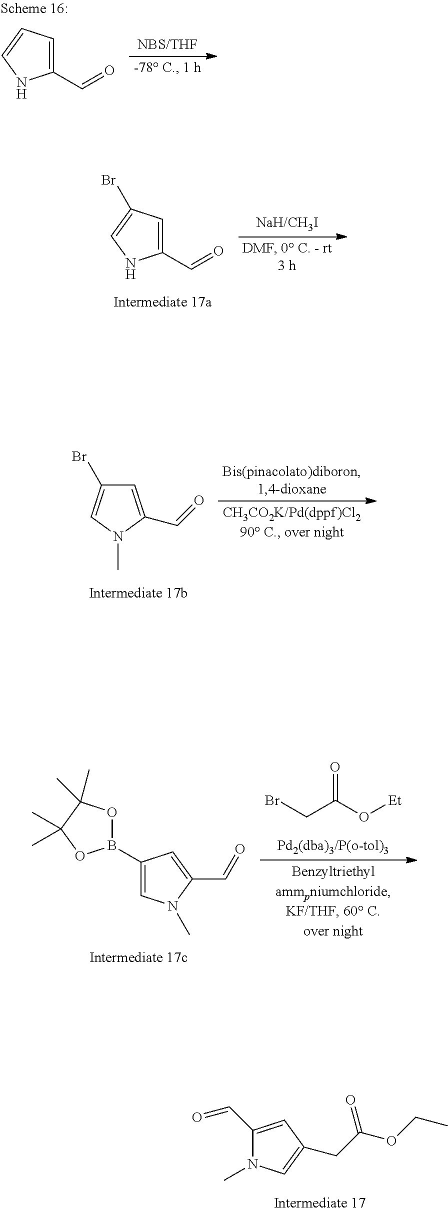

### Intermediate 17a: 4-Bromo-1H-pyrrole-2-carbaldehyde

To a 100 mL 3-neck RB flask charged with 1H-pyrrole-2-carbaldehyde (1.0 g, 10.5 mmol) in THF (20 mL) at −78° C., NBS (1.87 g, 10.53 mmol) in THF (20 mL) was added slowly and then the flaskw as maintained at the same temperature for 1 h. The reaction mixture was diluted with hexane and water mixture, the organic layer was decanted. The organic layer was dried over anhydrous Na₂SO₄ and then concentrated at low temperature under vacuo. It was recrystallized with n-hexane to yield the title compound (1.0 g, 54.0%) as a pale pink solid.

### Intermediate 17b: 4-Bromo-1-methyl-1H-pyrrole-2-carbaldehyde

To a 100 mL RB flask charged with NaH (0.28 g, 11.49 mmol) and DMF (5 mL) at 0° C., 4-bromo-1H-pyrrole-2-carbaldehyde (1.0 g, 5.75 mmol) in DMF (5.0 mL) was added drop wise over a period of 20 min. After 10 min, methyl iodide (2.45 g, 3.0 eq) was added and stirred at room temperature and the progress of reaction was monitored by TLC. The reaction mixture was quenched with ice and extracted with ethyl acetate. The organic layer was washed with water and brine, dried and concentrated to yield the title compound (0.25 g, 23.0%) as a colourless solid. ¹H NMR: (CDCl₃, 300 MHz) δ 9.43 (s, 1H), 6.80-6.82 (d, 2H), 3.87 (s, 3H).

### Intermediate 17c: 1-Methyl-4-(4,4,5,5-tetramethyl-1,3,2-dioxaborolan-2-yl)-1H-pyrrole-2-carbaldehyde

A sealed tube was charged with 4-bromo-1-methyl-1H-pyrrole-2-carbaldehyde (0.24 g, 1.28 mmol) and 1,4 dioxane (2 mL). To the above stirred solution bis(pinocolate)-diboran (0.39 g, 1.53 mmol) and potassium acetate (0.25 g, 2.55 mmol) was added and the tube was degasified with nitrogen after which Pd(dppf)Cl₂ (0.21 g, 0.0.26 mmol) was added and heated at 90° C. for over night. The reaction mixture was diluted with water and extracted with ethyl acetate; the organic layer was washed with water, brine solution and concentrated. The product was purified by combiflash to yield the title product (0.11 g, 36.0%) as brown oil. LCMS: (M+H)⁺=236.0; ¹H NMR: (CDCl₃, 300 MHz) δ 9.49 (s, 1H), 7.19 (s, 2H), 3.88 (s, 3H), 1.24 (s, 12H).

### Intermediate 17: Ethyl (5-formyl-1-methyl-1H-pyrrol-3-yl)acetate

A 100 mL sealed tube was charged with 1-methyl-4-(4,4,5,5-tetramethyl-1,3,2-dioxaborolan-2-yl)-1H-pyrrole-2-carbaldehyde (0.11 g, 0.47 mmol) and dry THF (3 mL). To the above solution Pd₂(dba)₃ (0.013 g, 0.041 mmol)o-tolyl)phosphino (0.013 g, 0.042 mmol), benzyltriethylammonium chloride (0.011 g, 0.047 mmol) and potassium fluoride (0.081 g, 1.4 mmol) was added. Then, ethyl bromoacetate (0.12 g, 0.7 mmol) was added and the resulting mixture was again degasified for 10 min. Then, it was heated at 75° C. over night. The reaction mixture was diluted with water and extracted with ethyl acetate, the organic layer was washed with water, brine and concentrated. The product was purified by preparative-TLC to yield the title compound (0.015 g, 15.0%) as brown oil. LCMS: (M+H)⁺=196.0

### Intermediate 18: 4-Formyl-1H-pyrrole-2-carboxylic acid

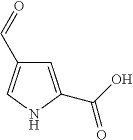

The intermediate 18 was synthesized from intermediate 11a by following the similar procedure described for intermediate 16 (0.065 g, 72.2%) as a beige solid. ¹H NMR: (DMSO-d₆, 300 MHz) δ 12.83 (s, 1H), 12.54 (s, 1H), 9.75 (s, 1H), 7.77-7.78 (m, 1H), 7.07 (s, 1H).

### Intermediate 19: (2′-Hydroxybiphenyl-3-yl)boronic acid

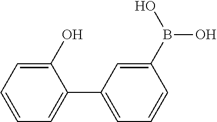 [SMILES: OB(O)c1cccc(-c2ccccc2O)c1]

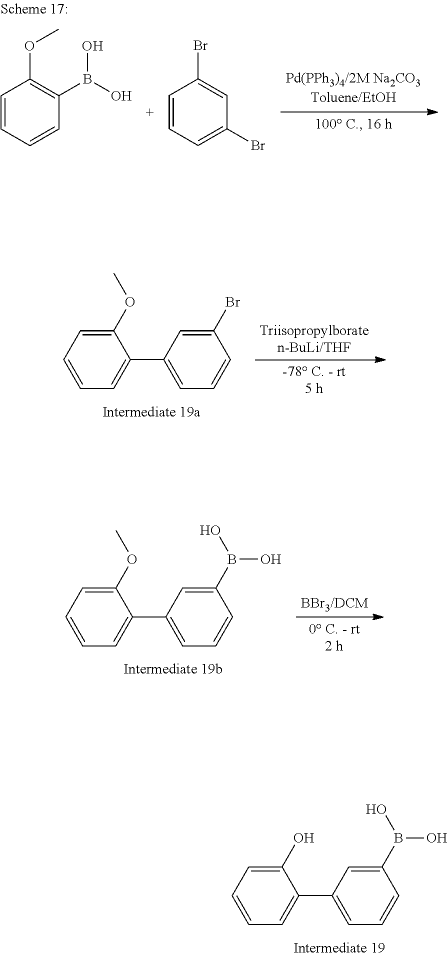 [SMILES: COc1ccccc1B(O)O ; Brc1cccc(Br)c1 ; COc1ccccc1-c1cccc(Br)c1 ; COc1ccccc1-c1cccc(B(O)O)c1 ; OB(O)c1cccc(-c2ccccc2O)c1]

### Intermediate 19a: 3′-Bromo-2-methoxybiphenyl

A 500 mL sealed tube was charged with 2-methoxyboronic acid (5.0 g, 1.0 eq), and 1,3-dibrormobenzene (7.76 g, 1.0 eq). To the stirred solution 2M Na₂CO₃ solution (153.0 mL), mixture of toluene (20 mL) and ethanol (20 mL) was added and the resulting solution was degasified with nitrogen for about 30 min. Then, Pd(PPh₃)₄ (1.9 g, 0.05 eq) was added and once again de-gasified with nitrogen and resulting mixture was refluxed at 80° C. over night. The reaction mixture was filtered through celite and the obtained crude product was purified by combiflash to obtain title product (5.0 g, 57.76%) as a gummy gel mass.

### Intermediate 19b: (2′-Methoxybiphenyl-3-yl)boronic acid

To a stirred solution of 3′-Bromo-2-methoxybiphenyl (3.0 g, 1.0 eq) in dry THF (100 mL) at −78° C., n-BuLi 15.2 mL (2.5M, 2.0 eq) was added drop wise under nitrogen atmosphere and stirred at the same temp for 1 h. Then, a solution of triisopropyl borate (8.8 mL, 2.0 eq) was added drop wise and the stirring was continued at room temperature for 1 h. The reaction mixture was diluted with water and extracted with ethyl acetate; concentrated to yield the title product (3.0 g, 69.24%) as an off white solid, which was taken as such for next step.

### Intermediate 19: (2′-Hydroxybiphenyl-3-yl)boronic acid

In a 100 mL two necked RB flask fitted with magnetic stirrer BBr₃ (0.45 mL, 4.6 mmol, 1.5 eq) was added to a −5° C. cooled solution of (2′-methoxybiphenyl-3-yl)boronic acid (0.7 g, 3.06 mmol, 1.0 eq) in DCM (10 mL), under nitrogen atmosphere for about 20 min. The reaction mixture was stirred with methanol, concentrated and crude portioned between water and ethyl acetate, the organic layer was washed brine solution and dried. The organic layer was concentrated under reduced pressure. It was purified by crystallization with hexane to yield the title product (0.284 g, 42.80%) as an off-white solid.

### Intermediate C: 5-(Biphenyl-4-yl)-6-chloro-1,3-dihydro-2H-indol-2-one

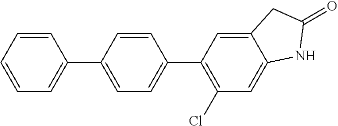 [SMILES: O=C1Cc2cc(-c3ccc(-c4ccccc4)cc3)c(Cl)cc2N1]

### Intermediate 20a: 4-Bromobiphenyl

This intermediate has been synthesized from 1,4-dibromo benzene (5.0 g, 1.0 eq) and phenyl boronic acid (2.6, 1.0 eq) by following the similar procedure for Intermediate 2a. It was obtained (3.3 g, 67.3%) as a white solid. ¹H NMR: (CDCl₃, 300 MHz) δ 7.47-7.51 (m, 4H), 7.37-7.40 (d, 3H), 7.29-7.35 (m, 2H).

### Intermediate 20: Biphenyl-4-ylboronic acid

This intermediate has been synthesized from 1, 4-dibromobenzene (3.3 g, 1.0 eq) and triisopropylborate (2.0 eq) by following the similar procedure for intermediate 2b. It was obtained (1.3 g, 46.59%) as a white solid. ¹H NMR: (DMSO-d₆, 300 MHz) δ 8.08 (br, 2H), 7.87-7.89 (d, 2H), 7.68-7.70 (d, 2H), 7.62-7.65 (d, 2H), 7.45-7.50 (t, 2H), 7.35-7.39 (t, 1H).

### Intermediate C: 5-(Biphenyl-4-yl)-6-chloro-1,3-dihydro-2H-indol-2-one

This intermediate has been synthesized from intermediate 3 (0.3 g, 1.0 eq) and intermediate 20a (0.25 g, 1.2 eq) by following the similar procedure for intermediate A. It was obtained (0.32 g, 83.55%) as beige solid. LCMS: (M+H)⁺=320.1; ¹H NMR: (CDCl₃, 300 MHz) δ 10.29 (s, 1H), 7.62-7.65 (d, 4H), 7.42-7.48 (m, 4H), 7.32-7.37 (m, 1H), 6.70 (s, 1H), 3.48 (s, 2H).

### Representative Procedures

### Example 1

### (5-{(E/Z)-[6-Chloro-5-(2′-hydroxybiphenyl-4-yl)-2-oxo-1,2-dihydro-3H-indol-3-ylidene]methyl}-2-methylphenyl)acetic acid

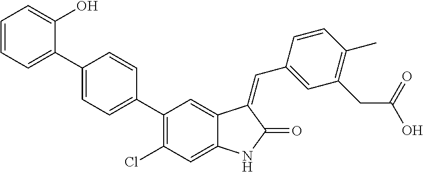 [SMILES: Cc1ccc(/C=C2\C(=O)Nc3cc(Cl)c(-c4ccc(-c5ccccc5O)cc4)cc32)cc1CC(=O)O]

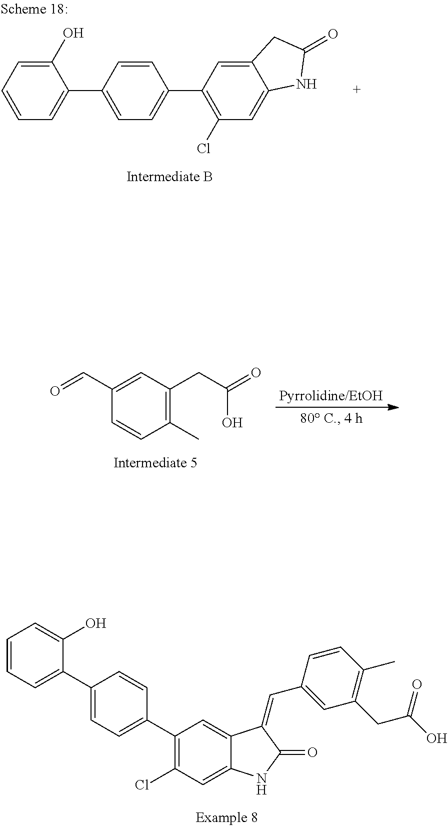 [SMILES: O=C1Cc2cc(-c3ccc(-c4ccccc4O)cc3)c(Cl)cc2N1 ; Cc1ccc(C=O)cc1CC(=O)O ; Cc1ccc(/C=C2\C(=O)Nc3cc(Cl)c(-c4ccc(-c5ccccc5O)cc4)cc32)cc1CC(=O)O]

To a solution of intermediate B (1.0 eq) and intermediate 5 (1.0 eq) in ethanol (4 mL), pyrrolidine (2.0 eq) was added and heated to 80° C. for 4 h. The reaction mixture was concentrated; the obtained residue was partitioned between water and ethyl acetate. The combined organic layer was washed with water and dried over anhydrous Na₂SO₄ The solvent was removed under vacuo, the obtained crude product, which was purified by column chromatography to yield the title compound (0.018 g, 12.10%) as a yellow solid. LCMS: (M−H)⁺=494.0

### Example 12

### (5-{(E/Z)-[6-Chloro-5-(2′-hydroxybiphenyl-4-yl)-2-oxo-1,2-dihydro-3H-indol-3-ylidene]methyl}-2-hydroxyphenyl)acetic acid

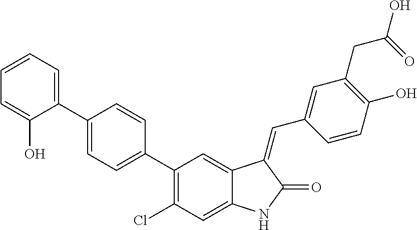 [SMILES: O=C(O)Cc1cc(/C=C2\C(=O)Nc3cc(Cl)c(-c4ccc(-c5ccccc5O)cc4)cc32)ccc1O]

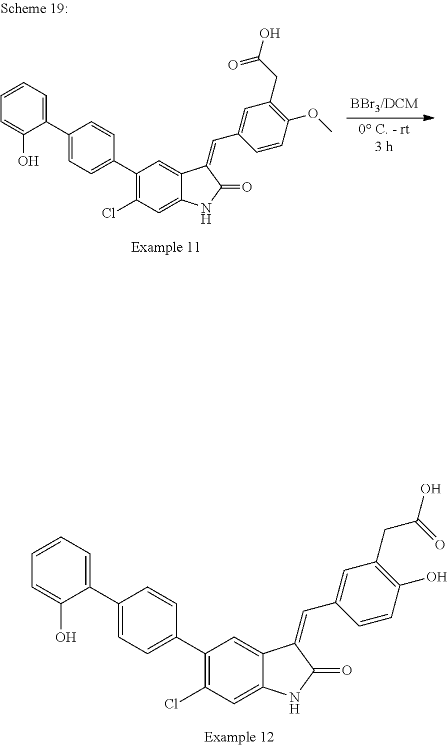 [SMILES: COc1ccc(/C=C2\C(=O)Nc3cc(Cl)c(-c4ccc(-c5ccccc5O)cc4)cc32)cc1CC(=O)O ; O=C(O)Cc1cc(/C=C2\C(=O)Nc3cc(Cl)c(-c4ccc(-c5ccccc5O)cc4)cc32)ccc1O]

To a stirred solution of Example 11 (0.05 g, 0.09 mmol) in DCM (5 mL) at 0° C., BBr₃ (0.122 g, 0.48 mmol) was added drop wise and maintained at same temperature for 30 min and stirred at room temperature for 5 h. The reaction mixture was quenched with ice and extracted with ethyl acetate and washed with water and brine solution and dried and concentrated. The obtained crude product was purified by column chromatography to yield the title product (2.0 mg, 4.1%) as a yellow solid. LC-MS: (M+H)⁺=496.0.

### Example 21

### Sodium Salt

Sodium 3-{(E/Z)-[6-chloro-5-(2′-hydroxybiphenyl-4-yl)-2-oxo-1,2-dihydro-3H-indol-3-ylidene]methyl}-1-methyl-1H-pyrazole-5-carboxylate A 25 mL RB flask was charged with 3-{(Z)-[6-chloro-5-(2′-hydroxybiphenyl-4-yl)-2-oxo-1,2-dihydro-3H-indol-3-ylidene]methyl}-1-methyl-1H-pyrazole-5-carboxylic acid (25.7 mg, 1.0 eq) in MeOH (0.5 mL) and THF (0.3 mL) mixture. To the above stirred solution NaOH solution (1M solution, 1,0 eq) was added drop wise at 0° C. Once the colour of the resulting mixture changed from yellow to brick red, the mixture was stirred at room temperature for 10 min. The reaction mixture was concentrated at vacuo. The obtained gummy material was evaporated along with methanol. The product was recrystallized with EtOAc and n-Hexane to title compound (0.015 g, 57.0%) as a brick red coloured solid.

### Example 24

### (3Z)-6-Fluoro-5-(2′-hydroxybiphenyl-4-yl)-3-(1-{3-[2-oxo-2-(pyrrolidin-1-yl)ethyl]phenyl}ethylidene)-1,3-dihydro-2H-indol-2-one

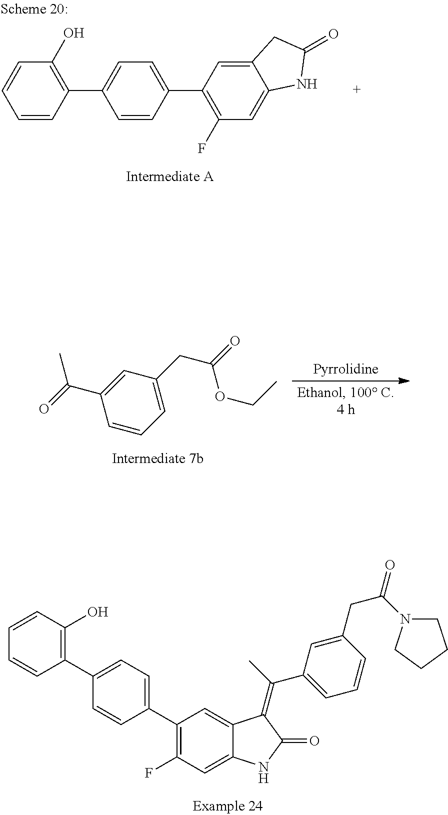 [SMILES: O=C1Cc2cc(-c3ccc(-c4ccccc4O)cc3)c(F)cc2N1 ; CCOC(=O)Cc1cccc(C(C)=O)c1 ; C/C(=C1/C(=O)Nc2cc(F)c(-c3ccc(-c4ccccc4O)cc3)cc21)c1cccc(CC(=O)N2CCCC2)c1]

To a solution of intermediate A and intermediate 7b (0.077 g, 0.4 mmol) in ethanol (3 mL), pyrrolidine (0.053 g, 0.7 mmol) was added and the reaction mixture was stirred at 80° C. over night. The reaction mixture was concentrated to dryness; obtained residue was acidified with 1N HCl, extracted with ethyl acetate. The combined organic layer was washed with water and dried over anhydrous Na₂SO₄. The solvent was removed under vacuo, the obtained crude product was purified by preparative HPLC to yield the title compound (0.003 g) as a pale brown solid. LCMS: (M−N+530.3; ¹H NMR (300 MHz, DMSO-d₆): δ 10.82 (s, 1H), 9.56 (s, 1H), 7.46-7.51 (m, 3H), 7.34-7.36 (m, 1H), 7.23-7.26 (m, 3H), 7.14-7.15 (d, 1H), 7.13 (br, 1H), 6.85-6.95 (m, 3H), 6.68-6.72 (d, 1H), 6.04-6.07 (d, 1H), 3.69 (s, 2H), 3.19-3.21 (d, 3H), 2.71 (s, 8H).

TABLE 1
Examples 1 to 24 were prepared by adopting Scheme 18.

| | | Interme- | |
|---|---|---|---|
| SI. No. | Structure | diates | Physical Data |
| 1 | 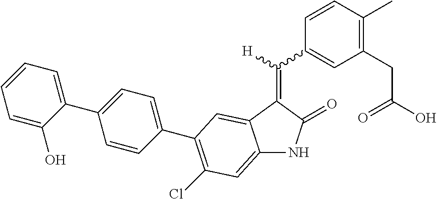 | B and 5 | LCMS: 494.0 (M − H)⁺ HPLC Rt: 15.80 and 16.75 min (Mixture) |
| 2 | 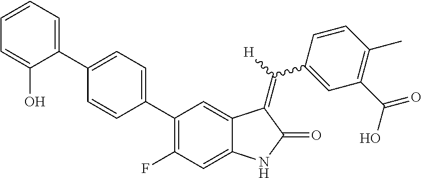 | A and 4 | LCMS: 464.1 (M + H)⁺ HPLC Rt: 14.62 and 15.31 min (Mixture) |
| 3 | 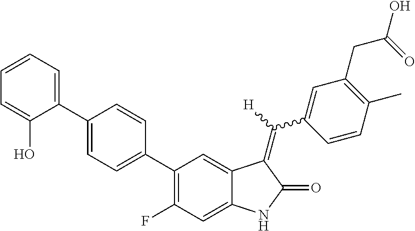 | A and 5 | LCMS: 478.0 (M − H)⁺ HPLC Rt: 15.14 min |
| 4 | 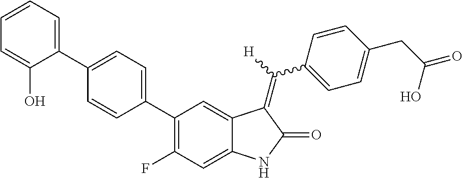 | A and 6 | LCMS: 464.1 (M − H)⁺ |
| 5 | 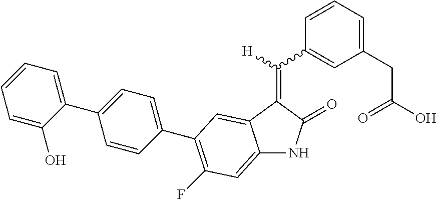 | A and 7 | LCMS: 464.0 (M − H)⁺ HPLC Rt: 15.62 min |
| 6 | 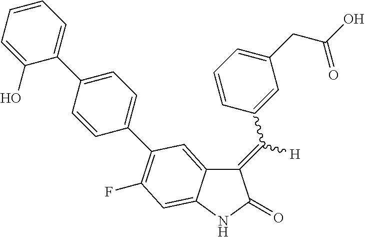 | A and 7 | LCMS: 464.1 (M − H)⁺ HPLC Rt: 14.74 min |
| 7 | 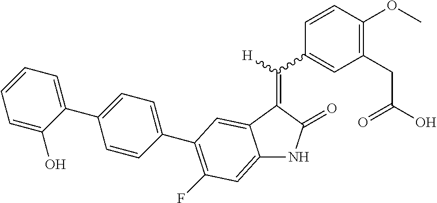 | A and 8 | LCMS: 494.1 (M − H)⁺ HPLC Rt: 14.78 and 15.78 min (Mixture) |
| 8 | 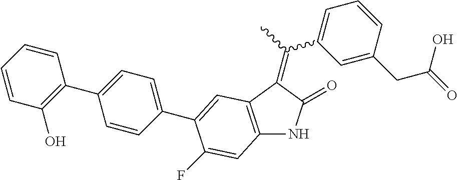 | A and 9 | LCMS: 478.1 (M − H)⁺ HPLC Rt: 15.20 min |
| 9 | 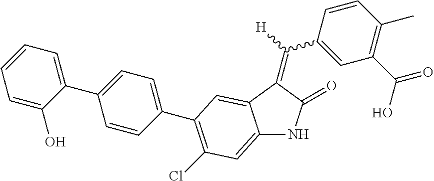 | B and 4 | LCMS: 480.0 (M − H)⁺ HPLC Rt: 16.04 and 16.91 min (Mixture) |
| 10 | 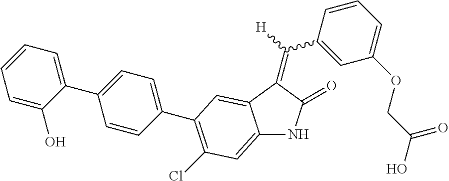 | B and 10 | LCMS: 496.0 (M − H)⁺ HPLC Rt: 15.01 and 16.19 min (Mixture) |
| 11 | 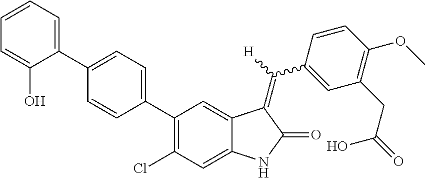 | B and 8 | LCMS: 510.0 (M− H)⁺ HPLC Rt: 15.23 and 16.27 min (Mixture) |
| 12 | 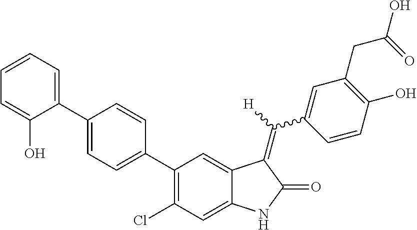 | B and 8 | LCMS: 496.0 (M − H)⁺ HPLC Rt: 13.84 and 14.90 min (Mixture) |
| 13 | 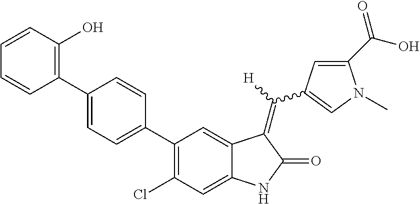 | B and 11 | LCMS: 469.0 (M − H)⁺ HPLC Rt: 14.42 and 15.63 min (Mixture) |
| 14 | 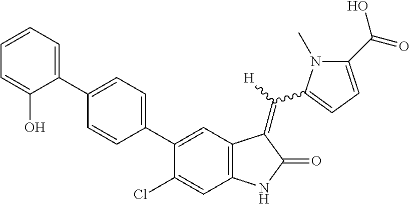 | B and 12 | LCMS: 469.0 (M − H)⁺ HPLC Rt: 14.79 and 15.78 min (Mixture) |
| 15 | 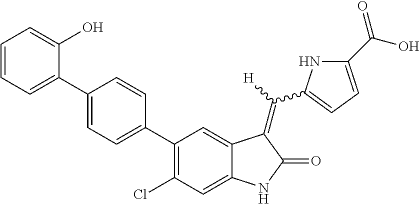 | B and 13 | LCMS: 455.0 (M − H)⁺ HPLC Rt: 16.36 min |
| 16 | 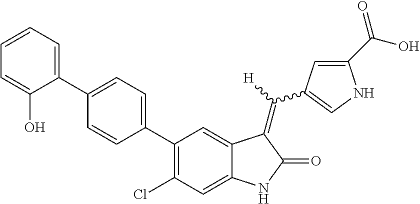 | B and 18 | LCMS: 455.0 (M − H)⁺ HPLC Rt: 13.51 and 14.64 min (Mixture) |
| 17 | 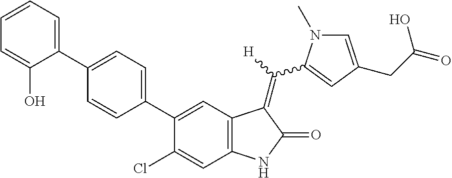 | B and 17 | LCMS: 483.0 (M − H)⁺ HPLC Rt: 14.24 and 15.19 min (Mixture) |
| 18 | 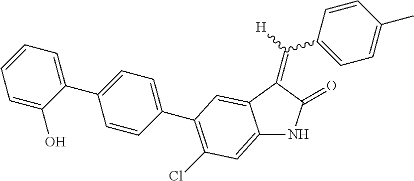 | B and 4-methyl- benzal- dehyde | LCMS: 438.0 (M + H)⁺ HPLC Rt: 18.72 and 19.69 min (Mixture) |
| 19 | 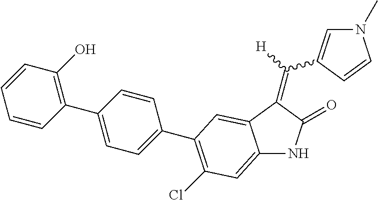 | B and 14 | LCMS: 427.0 (M + H)⁺ HPLC Rt: 16.10 and 17.16 min (Mixture) |
| 20 | 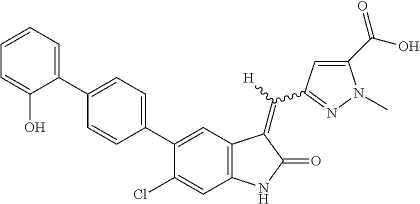 | B and 15 | LCMS: 470.1 (M − H)⁺ HPLC Rt: 13.14 min |
| 21 | 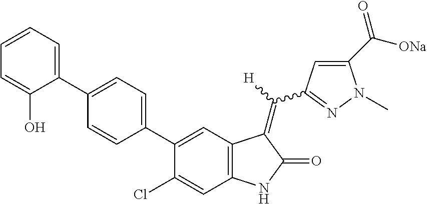 | B and 15 | LCMS: 470.0 (M-22 − H)⁺ HPLC Rt: 13.16 and 14.49 min (Mixture) |
| 22 | 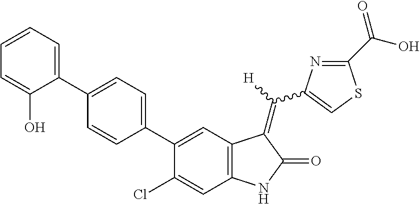 | B and 16 | LCMS: 473.2 (M − H)⁺ HPLC Rt: 14.63 min |
| 23 | 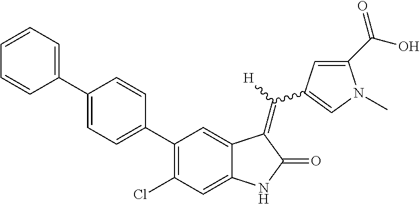 | C and 11 | LCMS: 453.0 (M − H)⁺ HPLC Rt: 16.77 and 18.42 min |
| 24 | 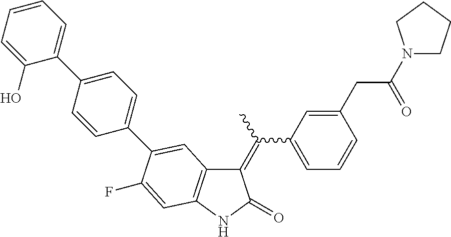 | A and 7b | LCMS: 531.3 (M − H)⁺ HPLC Rt: 15.81 min |

### Intermediate 21a: Methyl 1-ethyl-4-formyl-1H-pyrrole-2-carboxylate

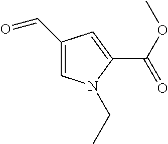

This intermediate has been synthesized from intermediate 11a (1.0 g, 6.5 mmol) and C₂H₅I (1.52 mL, 1.5 eq) by using the similar procedure described for intermediate 11b. It was obtained (0.8 g, 67.69%) as a red colour solid. ¹H NMR: (CDCl₃, 300 MHz) δ 9.70 (s, 1H), 7.40 (s, 1H), 7.30 (s, 1H), 4.31-4.38 (q, 2H), 3.78 (s, 3H), 1.36-1.41 (t, 3H).

### Intermediate 21: 1-Ethyl-4-formyl-1H-pyrrole-2-carboxylic acid

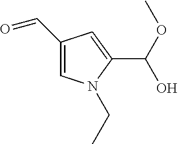

This intermediate has been synthesized from intermediate 21a (1.0 g, 6.5 mmol) by using the similar procedure described for intermediate 11. It was obtained (0.1 g, 35.29%) as a pale yellow colour solid. ¹H NMR: (DMSO-d₆, 300 MHz) δ 12.76 (s, 1H), 9.70 (s, 1H), 7.93-7.94 (d, 1H), 7.18-7.19 (d, 1H), 4.33-4.40 (q, 2H), 1.30-1.35 (t, 3H).

### Intermediate 22: (2′-{[2-(Trimethylsilyl)ethoxy]methoxy}biphenyl-4-yl)boronic acid

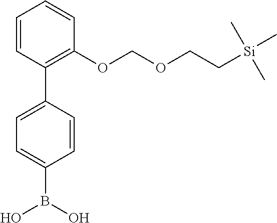 [SMILES: C[Si](C)(C)CCOCOc1ccccc1-c1ccc(B(O)O)cc1]

To a stirred solution of NaH (0.335 g, 14.0 mmol) in dry THF (20 mL) and dry DMF (10 mL) at 0° C., (2′-hydroxybiphenyl-4-yl)boronic acid (1.17 g, 5.5 mmol) in dry THF (5 mL) was added under nitrogen atmosphere. After 30 min, 2-(chloromethoxy)ethylytrimethyl)silane (2.32 g, 13.9 mmol) was added slowly and allowed to stir at 0° C. to room temperature for 1 h. The reaction mixture was quenched with ice and extracted by using ethyl acetate. The organic layer was washed with water and dried over anhydrous Na₂SO₄. The organic layer was concentrated under vacuo. The obtained crude product was purified by combi-flash to yield the title compound (0.55 g, 29.09%) as a colourless liquid.

### Intermediate 23: 6-Chloro-5-(2′-{[2-(trimethylsilyl)ethoxy]methoxy} biphenyl-4-yl)-1,3-dihydro-2H-indol-2-one

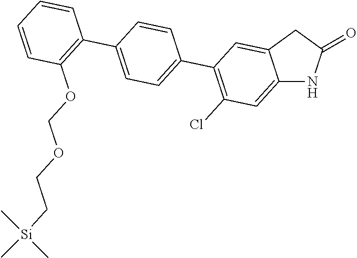 [SMILES: C[Si](C)(C)CCOCOc1ccccc1-c1ccc(-c2cc3c(cc2Cl)NC(=O)C3)cc1]

To a 100 mL sealed tube charged with intermediate 3 (0.367 g, 1.6 mmol), intermediate 22 (0.55 g, 1.6 mmol), potassium phosphate tribasic (0.89 g, 4.0 mmol) 1,2-dimethoxy-ethane (6 mL) and water (2 mL). The reaction mixture was degasified with nitrogen for 15 min, Pd (PPh₃)₄ (0.09 g, 0.079 mmol) was added and heated at 90° C. for 2 days. The reaction mixture was quenched with 1N HCl and extracted by using ethyl acetate. The combined organic layer was dried and concentrated. The product was purified by combi-flash to yield the title compound (0.46 g, 62.5%) as a pale white solid.

### Intermediate 24: (3Z)-6-Chloro-3-[(1-methyl-1H-pyrrol-3-yl)methylidene]-5-(2′-{[2-(trimethylsilyl)ethoxy]methoxy}biphenyl-4-yl)-1,3-dihydro-2H-indol-2-one

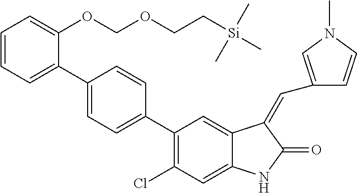 [SMILES: Cn1ccc(/C=C2\C(=O)Nc3cc(Cl)c(-c4ccc(-c5ccccc5OCOCC[Si](C)(C)C)cc4)cc32)c1]

To a solution of intermediate 23 (0.25 g, 0.5 mmol) and 1-methyl-1H-pyrrole-3-carbaldehyde (0.058 g, 0.5 mmol) in ethanol (3 mL), pyrrolidine (0.07 g, 2.0 eq) was added and stirred at room temperature and the resulting mixture was stirred at room temperature for 30 min. The reaction was quenched with ice and extracted with ethyl acetate, organic layer was washed with water and brine, dried over anhydrous Na₂SO₄ and concentrated to yield the title compound (0.27 g, 96.9%) as a yellow solid.

### Intermediate 25: Ethyl [(3Z)-6-chloro-3-[(1-methyl-1H-pyrrol-3-yl)methylidene]-2-oxo-5-(2′-{[2-(trimethylsilyl)ethoxy]methoxy}biphenyl-4-yl)-2,3-dihydro-1H-indol-1-yl]acetate

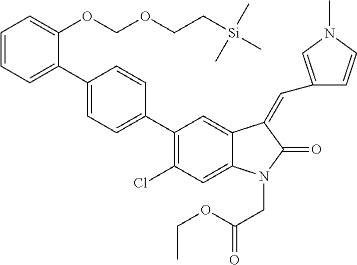 [SMILES: CCOC(=O)CN1C(=O)/C(=C\c2ccn(C)c2)c2cc(-c3ccc(-c4ccccc4OCOCC[Si](C)(C)C)cc3)c(Cl)cc21]

To a suspension of NaH (0.017 g, 0.71 mmol) in DMF (2 mL), a solution of intermediate 24 (0.27 g, 0.48 mmol) in DMF (1 mL) was added at 0° C. Then the resulting suspension was stirred at room temperature for 30 min, at 0° C. before adding ethyl bromoacetate (0.08 g, 0.5 mmol) to it after which it was further stirred at room temperature for 1 h. The reaction mixture was quenched with ice and extracted by using ethyl acetate. The organic layer was washed with water and dried over anhydrous Na₂SO₄. The organic layer was concentrated under vacuo. The product was purified by combi-flash to yield the title compound (0.3 g, 93.2%) as a yellow solid.

### Intermediate 26: Ethyl {(3Z)-6-chloro-5-(2′-hydroxybiphenyl-4-yl)-3-[(1-methyl-1H-pyrrol-3-yl)methylidene]-2-oxo-2,3-dihydro-1H-indol-1-yl}acetate

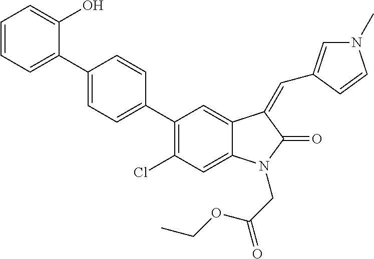 [SMILES: CCOC(=O)CN1C(=O)/C(=C\c2ccn(C)c2)c2cc(-c3ccc(-c4ccccc4O)cc3)c(Cl)cc21]

To a solution of intermediate 25 (0.2 g, 0.31 mmol) in THF (5 mL), conc. HCl was added and stirred at room temperature for 1 h. The reaction mixture was quenched with ice and extracted with ethyl acetate; the organic layer was washed with water and dried over anhydrous Na₂SO₄. The organic layer was concentrated under vacuo. The product was purified by combi-flash to yield the title compound (0.79 g, 100.0) as brown oil.

### Example 27

### {(3E/32)-6-Chloro-5-(2′-hydroxybiphenyl-4-yl)-3-[(1-methyl-1H-pyrrol-3-yl)methylidene]-2-oxo-2,3-dihydro-1H-indol-1-yl}acetic acid

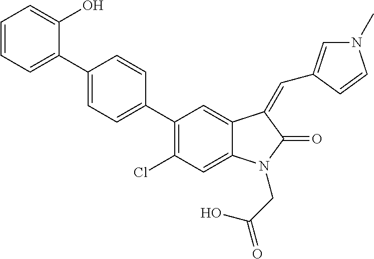 [SMILES: Cn1ccc(/C=C2\C(=O)N(CC(=O)O)c3cc(Cl)c(-c4ccc(-c5ccccc5O)cc4)cc32)c1]

To a solution of intermediate 26 (0.2 g, 0.4 mmol) in THF (5 mL), aqueous NaOH (dissolved in 1 mL of water) (0.04 g, 3.0 eq) added and the reaction mixture was stirred at room temperature for 1 h. The reaction mixture was concentrated and the obtained salt was washed with ether, and diluted with water and then neutralized with 1N HCl, then extracted with ethyl acetate, organic layer was washed with water and brine, dried over anhydrous Na₂SO₄ and concentrated. The product was purified by preparative-TLC to yield the title compound (0.02 g, 10.0%) as a yellow solid.

TABLE 2
Examples 25 to 29 were prepared by methods analogous to that described for Example 25.

| | | Interme- | |
|---|---|---|---|
| SI. No. | Structure | diates | Physical Data |
| 25 | 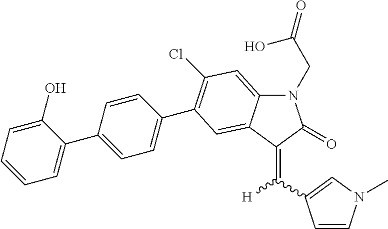 | 23 and 14 | LCMS: 485.0 (M + H)⁺ HPLC Rt: 15.57 and 15.59 min (Mixture) |
| 26 | 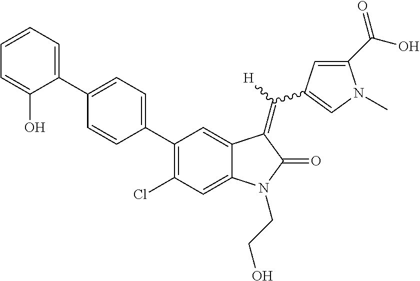 | 23 and 11 | LCMS: 515.0 (M + H)⁺ HPLC Rt: 14.11 and 15.33 min (Mixture) |
| 27 | 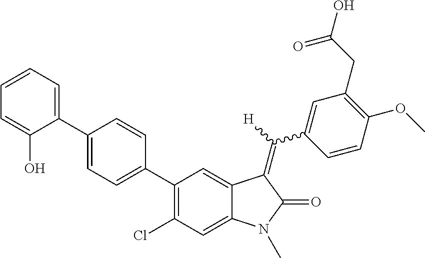 | 23 and 8 | LCMS: 524.0 (M − H)⁺ HPLC Rt: 17.16 and 18.20 min (Mixture) |
| 28 | 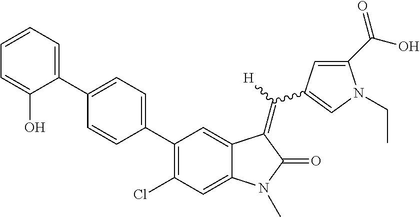 | 23 and 21 | LCMS: 497.1 (M − H)⁺ HPLC Rt: 17.16 and 18.46 min (Mixture) |
| 29 | 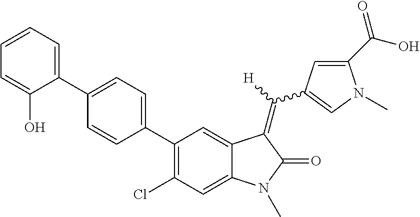 | 23 and 11 | LCMS: 483.0 (M − H)⁺ HPLC Rt: 16.45 and 17.74 min (Mixture); ¹H NMR (DMSO, 300 MHz, mixture of E/Z): δ 12.61 (s, 1H), 9.61-9.69 (m, 1H), 8.23 (m, 1H), 7.16- 7.95 (m, 10H), 6.88-6.99 (m, 2H), 3.90-3.94 (m, 3H), 3.26-3.28 (m, 3H) |

### Intermediate 29: Ethyl {5-[(E/Z)-(6-fluoro-5-iodo-2-oxo-1,2-dihydro-3H-indol-3-ylidene)methyl]-2-methylphenyl}acetate

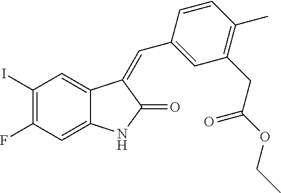 [SMILES: CCOC(=O)Cc1cc(/C=C2\C(=O)Nc3cc(F)c(I)cc32)ccc1C]

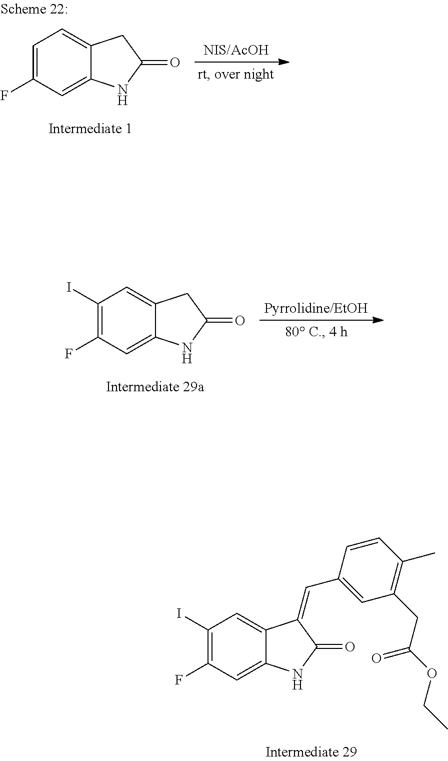 [SMILES: O=C1Cc2ccc(F)cc2N1 ; O=C1Cc2cc(I)c(F)cc2N1 ; CCOC(=O)Cc1cc(/C=C2\C(=O)Nc3cc(F)c(I)cc32)ccc1C]

### Intermediate 29a: 6-Fluoro-5-iodo-1,3-dihydro-2H-indol-2-one

To a solution of Intermediate 1 (3.0 g, 19.8 mmol) in acetic acid (60 mL, 1048.0 mmol), N-Iodo succinimide (4.47 g, 19.9 mmol) was added portion wise. It was allowed to stir at room temperature over night. The reaction mixture was diluted with water and extracted with ethyl acetate. The organic layer was washed with sodium thiosulphate solution, water and dried over anhydrous Na₂SO₄. The organic layer was concentrated under vacuo and the obtained crude product was purified to yield the title (2.0 g, 36.36%). LCMS: (M−H)⁺=275.9; ¹H NMR: (DMSO-d₆, 300 MHz) δ 10.60 (s, 1H), 7.57-7.59 (d, 1H), 6.70-6.73 (d, 1H), 3.46 (s, 2H).

### Intermediate 29: Ethyl {5-[(E/Z)-(6-fluoro-5-iodo-2-oxo-1,2-dihydro-3H-indol-3-ylidene)methyl]-2-methylphenyl}acetate

This intermediate was synthesized from intermediate 29a (1.3 g, 4.7 mmol) and intermediate 5b (1.2 g, 5.6 mmol) by using the similar procedure described for intermediate 24. It was obtained as a yellow solid (1.1 g, 51.06%). LCMS: (M−H)⁺=464.0

### Example 30

### AM_A_317

### (5-{(E/Z)-[6-Fluoro-5-(2′-hydroxybiphenyl-3-yl)-2-oxo-1, 2-dihydro-3H-indol-3-ylidene]methyl}-2-methylphenyl)acetic acid

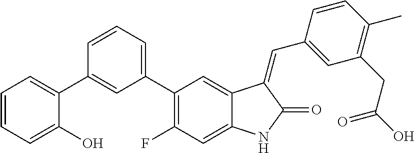 [SMILES: Cc1ccc(/C=C2\C(=O)Nc3cc(F)c(-c4cccc(-c5ccccc5O)c4)cc32)cc1CC(=O)O]

This compound has been synthesized by using intermediate 19 and intermediate 29 by following the similar procedure as described in intermediate A.

TABLE 3
Examples 30 was prepared by methods described above

| | | | Physical |
|---|---|---|---|
| SI. No. | Structure | Intermediates | Data |
| 30 | 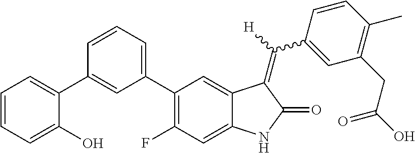 | 19 and 29 | LCMS: 480.1 (M + H)⁺ HPLC Rt: 15.25 min |

### Intermediate 32: 5-Bromo-6-chloro-1,3-dihydro-2H-pyrrolo[3,2-b]pyridin-2-one

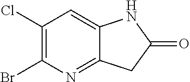 [SMILES: O=C1Cc2nc(Br)c(Cl)cc2N1]

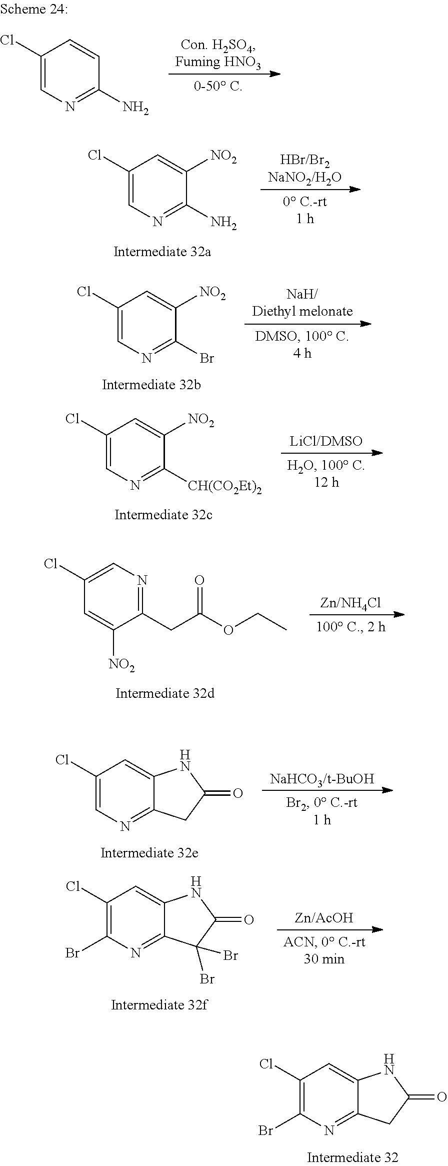

### Intermediate 32a: 5-Chloro-3-nitropyridin-2-amine

To a 250 mL 3 necked RK flask charged with conc.H₂SO₄ (30 mL) at 0° C., 5-chloropyridin-2-amine (12.5 g) was added portion wise and solution was allowed to stir for 1 h and heated at 50° C. to dissolve starting material completely. Then, conc. HNO₃ (8 mL) was added drop wise to the resulting solution through addition funnel. The reaction was monitored at every 10 minutes. After completion, 40% sodium hydroxide solution (PH=6-7) was added, the product was extracted with ethyl acetate, washed with water, and dried over anhydrous Na₂SO₄. The solvent was removed under vacuo to yield the product (11.0 g, 51.0%) as a pale green solid. LCMS: (M−H)⁺=172.9

### Intermediate 32b: 2-Bromo-5-chloro-3-nitropyridine

To a solution of intermediate 32a (10.0 g, 57.6 mmol) in HBr [(31.0 mL (100%), 286.4 mmol)] at 0° C., sodium nitrite (13.8 g, 199.9 mmol) was added drop wise. To the stirred solution Br₂ (10.0 mL, 197.1 mmol) in water was added and stirred at room temperature for 1 h. The reaction mixture was basified with NaHCO₃ solution (PH=7) and extracted with ethyl acetate, washed with water, and dried over anhydrous Na₂SO₄. The solvent was removed under vacuo to yield the title product (10.0 g, 73.0%) as a yellow solid.

### Intermediate 32c: Diethyl (5-chloro-3-nitropyridin-2-yl)propanedioate

To a solution of sodium hydride [(1.52 g, 63.17 mmol (95%)] in DMSO (20.0 mL) at 0° C., diethylmalonate (10.11 g, 63.17 mmol) was added and kept for reflux at 100° C. for 1 h. The reaction mixture was cooled to room temperature before drop wise addition of intermediate 32b (10.0 g, 42.11 mmol) in DMSO (20 mL). The resulting mixture was refluxed at 100° C. for 3 h. The reaction mixture was quenched with ice water and extracted by using Ethyl acetate washed with water, and dried over anhydrous Na₂SO₄. The solvent was removed under vacuo to yield the title compound (10.0 g, 75.00%) as a brown oily product. LCMS: (M−H)⁺=315.0

### Intermediate 32d: Ethyl (5-chloro-3-nitropyridin-2-yl) acetate

To a solution of intermediate 32c (10.0 g, 31.65 mmol) in DMSO (15 mL), Lithium chloride (4.02 g, 94.93 mmol) solution in water (5 mL) was added and stirred for over night at 100° C. The reaction mixture was quenched with ice water and extracted by using ethyl acetate washed with water, and dried over anhydrous Na₂SO₄. The solvent was removed under vacuo. The crude product was purified by combi-flash to yield the title product (5.2 g, 67.26%) as a pale orange solid. LCMS: (M−H)⁺=243.0; ¹H NMR: (CDCl₃, 300 MHz) δ 8.68-8.69 (d, 1H), 8.36-8.37 (d, 1H), 4.24 (s, 2H), 4.09-4.16 (s, 2H), 1.17-1.24 (t, 3H).

### Intermediate 32e: 6-Chloro-1,3-dihydro-2H-pyrrolo[3,2-b]pyridin-2-one

To a solution of intermediate 32d (5.0 g, 20.43 mmol) in ethanol/water [20 mL, (4:1) mixture], was added ammonium chloride (8.7 g, 163.5 mmol) and zinc powder (6.7 g, 102.2 mmol) and stirred at 100° C. for 2 days. The reaction mixture was filtered and concentrated. The compound was extracted with ethyl acetate washed with water, brine. The organic layer dried over Na₂SO₄, concentrated under vacuo. The crude product was purified by purified by column chromatography to yield the title compound (1.8 g, 52.0%). LCMS: (M+H)⁺=169.0

### Intermediate 32f: 3,3,5-Tribromo-6-chloro-1,3-dihydro-2H-pyrrolo[3,2-b]pyridin-2-one

To a solution of intermediate 32e (1.8 g, 10.7 mmol) in t-butanol (30 mL) at 0° C. saturated solution of NaHCO₃ was added and stirred for 10 min. To the reaction mixture Br₂ (10.27 g, 64.3 mmol) was added and stirred at room temperature for 1 h. The reaction mixture was quenched with ice water and extracted by using ethyl acetate washed with water, and dried over anhydrous Na₂SO₄. The solvent was removed under vacuo to yield the title product (4.33 g, 100.0%) as an orange solid.

### Intermediate 32: 5-Bromo-6-chloro-1,3-dihydro-2H-pyrrolo[3,2-b]pyridin-2-one

To a solution of intermediate 32f (0.4 g, 0.987 mmol) in acetonitrile (7 mL), acetic acid (3 mL) and zinc powder (0.645 g) was added at 0° C. and stirred at room temperature for 30 min. The reaction mixture was diluted with water and extracted with ethyl acetate. The organic layer was washed with water and dried over anhydrous Na₂SO₄. The crude product was purified by column chromatography to yield title compound (0.15 g, 60.6%) as pale yellow solid. LCMS: (M−H)⁺=246.7

### Intermediate G: 6-Chloro-5-(2′-hydroxybiphenyl-4-yl)-1,3-dihydro-2H-pyrrolo[3,2-b]pyridin-2-one

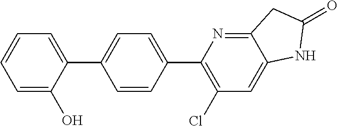 [SMILES: O=C1Cc2nc(-c3ccc(-c4ccccc4O)cc3)c(Cl)cc2N1]

This intermediate was synthesized from intermediate 32 (0.10 g, 1.0) and 2′-hydroxybiphenyl-4-yl)boronic acid (0.086 g, 1.0 eq) by following the similar procedure described for intermediate E. It was obtained as a yellow solid (0.053 g, 39.35%) LCMS: (M−H)⁺=335.0

### Intermediate 33: 2-Bromo-5-(2-methoxyphenyl)pyridine

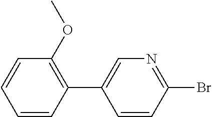

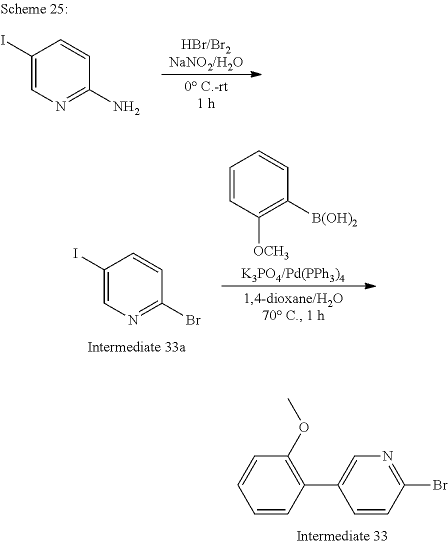

### Intermediate 33a: 2-Bromo-5-iodopyridine

To a solution of 5-iodopyridin-2-amine (0.7 g, 3.18 mmol) in HBr [(1.26 g, 15.6 mmol (48% in water)] at 0° C. sodium nitrite (0.746 g, 10.82 mmol) in water was added drop wise followed by addition of bromine (1.71 g, 10.82 mmol). The reaction mixture was at room temperature for 1 h. The reaction mixture was quenched with NaOH solution and extracted with ethyl acetate, washed with water, and dried over anhydrous Na₂SO₄ The solvent was removed under vacuo. The crude product was purified by column chromatography to yield title compound (0.6 g, 65.23%) as a white solid. LCMS: (M+H)⁺=284; ¹H NMR: (DMSO-d₆, 300 MHz) δ 8.64-8.65 (d, 1H), 8.09-8.12 (dd, 1H), 7.50-7.53 (m, 1H).

### Intermediate 33: 2-Bromo-5-(2-methoxyphenyl)pyridine

To the solution of (2-methoxyphenyl)boronic acid (0.471 g, 3.1 mmol) in 1,4-dioxane (20 mL) 2-bromo-5-iodopyridine (1.1 g, 3.87 mmol), potassium phosphate tribasic (2.46 g, 11.61 mmol) and water (5 mL) was added. The reaction mixture was degasified for 10 min under nitrogen, followed by addition of Pd(PPh₃)₄ (0.224 g, 0.194 mmol). The reaction mixture was heated at 70° C. for 1 h. The reaction mixture was diluted with water and extracted with ethyl acetate. The organic layer was washed with water and dried over anhydrous Na₂SO₄. The organic layer was concentrated under vacuo to yield crude product, which was purified by column chromatography to yield title compound (0.72 g, 87.09%) as a colourless liquid. LCMS: (M+H)⁺=264-266; ¹H NMR: (CDCl₃, 300 MHz) δ 8.43-8.44 (d, 1H), 7.65-7.68 (dd, 1H), 7.42-7.45 (d, 1H), 7.29-7.34 (t, 1H), 7.19-7.23 (m, 1H), 6.99-7.01 (d, 1H), 6.92-6.96 (m, 1H), 3.75 (s, 3H).

### Intermediate 34: 6-Chloro-5-(4,4,5,5-tetramethyl-1,3,2-dioxaborolan-2-yl)-1,3-dihydro-2H-indol-2-one

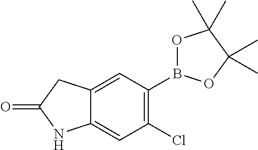 [SMILES: CC1(C)OB(c2cc3c(cc2Cl)NC(=O)C3)OC1(C)C]

This intermediate has been synthesized from intermediate 3 using the similar procedure used for intermediate 5a. It was obtained as a pale yellow solid (0.53 g, 90.28%). LCMS: (M−H)⁺=292.0

### Intermediate 35: 6-Chloro-5-[5-(2-methoxyphenyl)pyridin-2-yl]-1,3-dihydro-2H-indol-2-one

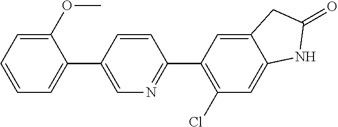 [SMILES: COc1ccccc1-c1ccc(-c2cc3c(cc2Cl)NC(=O)C3)nc1]

To a solution of intermediate 33 (0.3 g, 1.1 mmol) and intermediate 34 (0.33 g, 1.13 mmol) in 1,4-Dioxane (3 mL) and DMF (3 mL), 2-dicyclohexylphosphino-2,4,6-triisopropylbiphenyl (0.082 g, 0.17 mmol) was added. The reaction mixture was degasified for 15 min and then PdCl₂(PPh₃)₂ (0.04 g, 0.057 mmol) was added to it and the resulting mixture was heated at 100° C. over night. The reaction mixture was diluted with water and extracted with ethyl acetate. The organic layer was washed with water and dried over anhydrous Na₂SO₄. The organic layer was concentrated under vacuo to yield crude product, which was purified by column chromatography to yield title compound (0.18 g, 45.45%) as a pale brown solid. LCMS: (M−H)⁺=349.0; ¹H NMR: (DMSO-d₆, 300 MHz) δ 10.64 (s, 1H), 8.76 (s, 1H), 7.86-8.00 (dd, 1H), 7.68-7.71 (d, 1H), 7.49 (s, 1H), 7.40-7.44 (m, 2H), 7.16-7.19 (d, 1H), 7.06-7.11 (t, 1H), 6.95 (s, 1H), 3.82 (s, 3H), 3.55 (s, 2H).

### Intermediate H: 6-Chloro-5-[5-(2-hydroxyphenyl)pyridin-2-yl]-1,3-dihydro-2H-indol-2-one

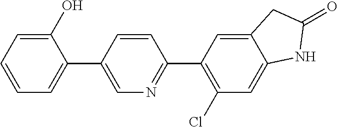 [SMILES: O=C1Cc2cc(-c3ccc(-c4ccccc4O)cn3)c(Cl)cc2N1]

This intermediate has been synthesized from intermediate 35 using the similar procedure used for intermediate 2. It was obtained as a pale brown solid (0.135 g, 100.0%). LCMS: (M+H)⁺=337.0; ¹H NMR: (DMSO-d₆, 300 MHz) δ 10.63 (s, 1H), 9.82 (s, 1H), 8.81-8.82 (d, 1H), 8.02-8.06 (dd, 1H), 7.68-7.70 (d, 1H), 7.49 (s, 1H), 7.37-7.40 (d, 1H), 7.21-7.26 (t, 1H), 6.98-7.01 (d, 1H), 6.91-6.96 (m, 2H), 3.56 (s, 2H).

### Intermediate I: 6-Chloro-5-[6-(2-hydroxyphenyl)pyridin-3-yl]-1,3-dihydro-2H-indol-2-one

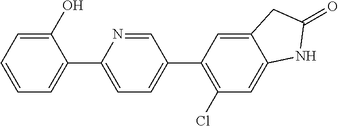 [SMILES: O=C1Cc2cc(-c3ccc(-c4ccccc4O)nc3)c(Cl)cc2N1]

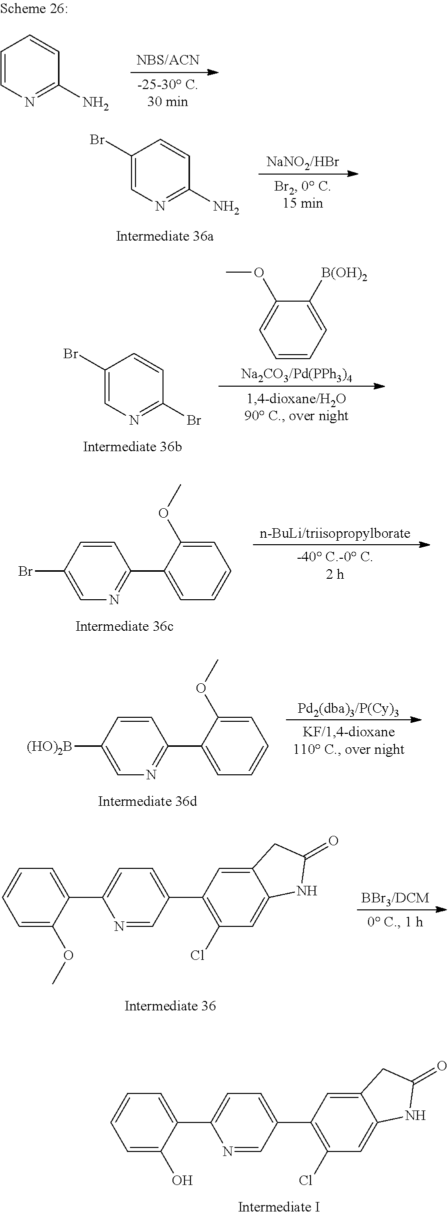

### Intermediate 36a: 5-Bromopyridin-2-amine

To a solution of pyridin-2-amine (10.0 g, 10.06 mmol) in acetonitrile (100 mL) at −30° C., NBS (18.9 g, 106.2 mmol) was added and it was stirred at −30° C. for 30 min. The reaction mixture was quenched with ice and extracted with ethyl acetate. The organic layer was washed with water and dried over anhydrous Na₂SO₄. The organic layer was concentrated under vacuo. The product was purified by column chromatography to yield title compound (10.0 g, 54.37%) as a yellow solid. LCMS: (M+2)⁺=175.0

### Intermediate 36b: 2, 5-Dibromopyridine

To a solution of 5-bromopyridin-2-amine (5.0 g, 28.9 mmol) in hydrogen bromide (50.0 mL, 46%), sodium nitrite (15.0 g, 217.4 mmol) solution in water was slowly added at 0° C. abd then stirred for 10 min at the same temperature. To the reaction mixture Br₂ (37.2 mL, 232.8 mmol) was slowly added at 0° C. and stirred for 15 min at 0° C. The reaction mixture was basified with sodium hydroxide and extracted with ethyl acetate, washed with sodium thiosulphate solution, water and brine solution. The organic layer was dried over anhydrous Na₂SO₄ and concentrated under vacuo. The product was purified by column chromatography to yield title compound (4.0 g, 58.0%) white solid. LCMS: (M+H)⁺=237.9; ¹H NMR: (DMSO-d₆, 300 MHz) δ 8.57-8.58 (d, 1H), 8.00-8.04 (dd, 1H), 7.64-7.67 (d, 1H).

### Intermediate 36c: 5-Bromo-2-(2-methoxyphenyl)pyridine

This intermediate has been synthesized from intermediate 36b and intermediate (2-methoxyphenyl) boronic acid by following the similar procedure for intermediate 2a. The title compound obtained (1.8 g, 81.0%) as pale yellow oil. ¹H NMR: (DMSO-d₆, 300 MHz) δ 8.77-8.78 (d, 1H), 8.05-8.08 (dd, 1H), 7.82-7.85 (d, 1H), 7.72-7.75 (dd, 1H), 7.41-7.46 (m, 1H),), 7.15-7.18 (d, 1H), 7.05-7.10 (t, 1H), 3.80 (s, 3H).

### Intermediate 36d: [6-(2-Methoxyphenyl)pyridin-3-yl]boronic acid

This intermediate was synthesized from intermediate 36c by following the procedure described for intermediate 27. It was obtained (0.05 g, 27.0%) as a pale yellow solid. LCMS: (M+H)⁺=230.1

### Intermediate 36: 6-Chloro-5-[6-(2-methoxyphenyl)pyridin-3-yl]-1,3-dihydro-2H-indol-2-one

This intermediate has been synthesized from intermediate 36d and intermediate 3 by following the similar procedure for intermediate E. It was obtained as a pale brown solid (0.1 g, 28.0%). LCMS: (M+H)⁺=351.0

### Intermediate I: 6-Chloro-5-[6-(2-hydroxyphenyl)pyridin-3-yl]-1,3-dihydro-2H-indol-2-one

This intermediate was synthesized from intermediate 36 by following the similar procedure described for intermediate 2. It was obtained (0.04 g, 40.0%) as a pale brown solid.

### Intermediate J: 6-Chloro-5-[4-(pyridin-4-yl)phenyl]-1,3-dihydro-2H-indol-2-one

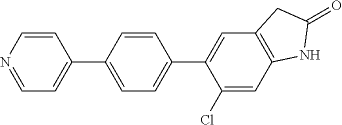 [SMILES: O=C1Cc2cc(-c3ccc(-c4ccncc4)cc3)c(Cl)cc2N1]

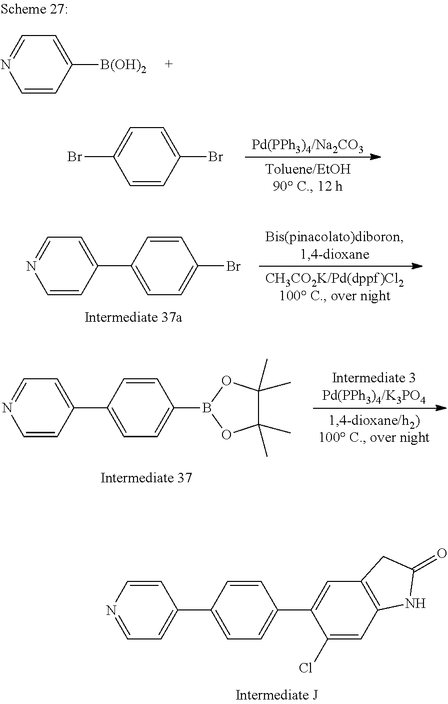 [SMILES: OB(O)c1ccncc1 ; Brc1ccc(Br)cc1 ; Brc1ccc(-c2ccncc2)cc1 ; CC1(C)OB(c2ccc(-c3ccncc3)cc2)OC1(C)C ; O=C1Cc2cc(-c3ccc(-c4ccncc4)cc3)c(Cl)cc2N1]

### Intermediate 37a: 4-(4-Bromophenyl)pyridine

A 500 mL sealed tube was charged with pyridin-4-ylboronic acid (1.6 g, 13.0 mmol) 1,4-dibromobenzene (3.07 g, 13.0 mmol), sodium carbonate (5 mL, 2M solution) and mixture of toluene (10 mL) and water (10 mL). The reaction mixture was purged with argon for 30 min. Then, Pd(PPh₃)₄ (0.75 g, 0.05 eq) was added to the reaction mixture and heated at 90° C. about 12 h. After cooling, the reaction mixture was extracted with ethyl acetate. The organic layer was washed with water and dried over anhydrous Na₂SO₄. The organic layer was concentrated under vacuo to yield crude product, which was purified by column chromatography to yield title compound (0.8 g, 26.26%) as a white solid. LCMS: (M+2)⁺=236.0; ¹H NMR: (DMSO-d₆, 300 MHz) δ 8.64-8.66 (d, 2H), 7.71-7.80 (m, 6H).

### Intermediate 37: 4-[4-(4,4,5,5-Tetramethyl-1,3-dioxolan-2-yl)phenyl]pyridine

A 100 mL sealed tube was charged with 4-(4-bromophenyl)pyridine (0.9 g, 3.8 mmol), bis(pinacolato)diboron (1.17 g, 4.61 mmol), potassium acetate (0.745 g, 7.6 mmol) and 1-4 dioxane (10 mL). The reaction mixture was purged with argon for 30 min. Then, Pd(dppf)Cl₂ (0.75 g, 0.05 eq) was added and heated at 100° C. over night. After cooing, the reaction mixture was extracted with ethyl acetate. The organic layer was washed with water and dried over anhydrous Na₂SO₄. The organic layer was concentrated under vacuo to yield crude product, which was purified by combiflash to yield title compound (1.0 g, 100.0%). LCMS: (M+H)⁺=282.1

### Intermediate J: 6-Chloro-5-[4-(pyridin-4-yl)phenyl]-1,3-dihydro-2H-indol-2-one

A 100 mL sealed tube charged with intermediate 37 (1.0 g, 3.5 mmol), intermediate 3 (0.86 g, 3.1 mmol), potassium phosphate tribasic (2.0 g, 10.5 mmol), 1, 4-dioxane (10 mL) and water (3 mL) was degassed with nitrogen for 15 min. To the above solution Pd(PPh₃)₄ (0.2 g, 0.05 eq) was added and heated at 100° C. over night. After cooling, the reaction mixture was diluted with water and extracted by using ethyl acetate. The combined organic layer was dried and concentrated. The product was purified by combi-flash to yield title compound (0.2 g, 18.1%) as a brown solid. LCMS: (M+H)⁺=321.0; ¹H NMR: (DMSO-d₆, 300 MHz) δ 10.61 (s, 1H), 8.66-8.67 (d, 2H), 7.88-7.91 (d, 2H), 7.77-7.78 (d, 2H), 7.53-7.56 (d, 2H), 7.30 (s, 1H), 6.97 (s, 1H), 3.54 (s, 2H).

### Intermediate K: 6-Fluoro-5-[4-(pyridin-4-yl)phenyl]-1,3-dihydro-2H-indol-2-one

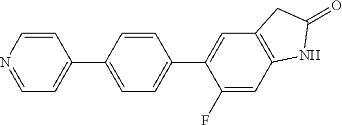 [SMILES: O=C1Cc2cc(-c3ccc(-c4ccncc4)cc3)c(F)cc2N1]

A microwave vial was charged with intermediate 1 (0.2 g, 0.9 mmol), intermediate 37 (0.24 g, 0.9 mmol) and DMF: 1,4-Dioxane: water (2 mL: 0.5 mL: 0.5 mL) mixture. To the above solution potassium phosphate (tribasic) (0.55 g, 1.8 mmol) was added and the vial was degasified for 15 min after which Pd(PPh₃)₄ (0.05 g, 0.03 eq) was added and heated at 150° C. for 30 min. After cooling, the reaction mixture diluted with water and extracted by using ethyl acetate. The combined organic layer was dried and concentrated. The product was purified by chromatography to yield title compound (0.075 g, 27.0%) as a brown solid. LCMS: (M+H)⁺=305.1

### Intermediate L: 6-Chloro-5-[5-(2-hydroxyphenyl)pyrimidin-2-yl]-1,3-dihydro-2H-indol-2-one

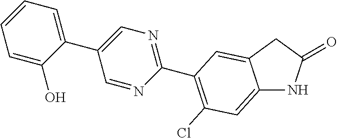 [SMILES: O=C1Cc2cc(-c3ncc(-c4ccccc4O)cn3)c(Cl)cc2N1]

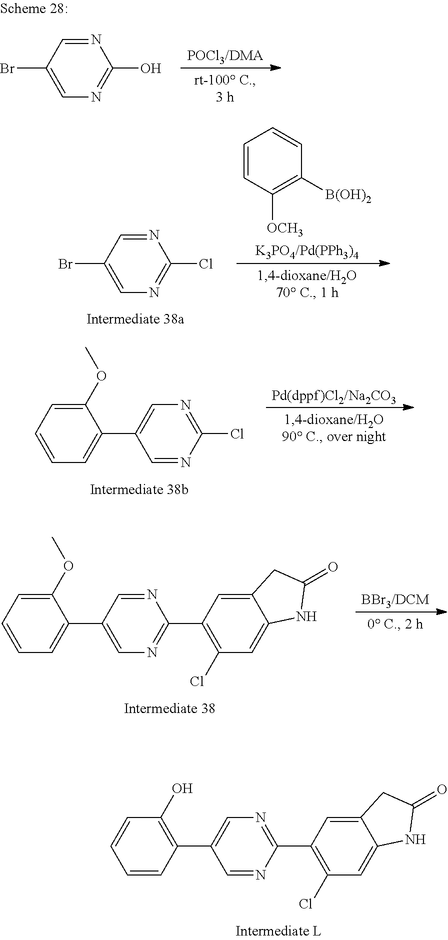 [SMILES: Oc1ncc(Br)cn1 ; COc1ccccc1B(O)O ; Clc1ncc(Br)cn1 ; COc1ccccc1-c1cnc(Cl)nc1 ; COc1ccccc1-c1cnc(-c2cc3c(cc2Cl)NC(=O)C3)nc1 ; O=C1Cc2cc(-c3ncc(-c4ccccc4O)cn3)c(Cl)cc2N1]

### Intermediate 38a: 5-Bromo-2-chloropyrimidine

To a stirred solution of 5-bromopyrimidin-2-ol (1.0 g, 1.0 eq) in POCl₃ (1 mL) dimethylaniline (0.21 g, 0.3 eq) was added under nitrogen atmosphere. Then, the reaction mixture was refluxed at 100° C. for 3 h. After cooling, the reaction mixture was basified with saturated sodium carbonate solution and extracted with ethyl acetate. The organic layer was then separated and dried over Na₂SO₄ and concentrated to get a crude which was then subjected to the combiflash-12 g silica and isolated the title product as an off white solid (0.67 g, 61.41%). ¹H NMR: (DMSO-d₆, 300 MHz) δ 9.00-9.01 (d, 2H).

### Intermediate 38b: 2-Chloro-5-(2-methoxyphenyl)pyrimidine

This intermediate was synthesized from intermediate 38a by following the similar procedure described for intermediate 33. It was obtained (0.8 g, 68.7%) as a white solid.

### Intermediate 38: 6-Chloro-5-[5-(2-methoxyphenyl)pyrimidin-2-yl]-1,3-dihydro-2H-indol-2-one

To a solution of 6-chloro-5-(4,4,5,5-tetramethyl-1,3,2-dioxaborolan-2-yl)-1,3-dihydro-2H-indol-2-one in 1,4-dioxan/water (3/1), intermediate 38b and sodium carbonate were added and degasified for 20 minutes. This was followed by addition of Pd(dppf)Cl₂ under nitrogen atmosphere. The mixture was heated to 90° C. for over night. After completion of the reaction extracted with ethyl acetate washed with water, brine, the organic layer was dried over sodium sulphate and concentrated. It was purified by column chromatography to yield the title product (0.16 g, 22.0%) as an off white solid. LCMS: (M+H)⁺=352.0; ¹H NMR: (DMSO-d₆, 300 MHz) δ 10.68 (s, 1H), 9.04 (s, 2H), 7.68 (s, 1H), 7.45-7.54 (m, 2H), 7.10-7.23 (m, 2H), 6.96 (s, 1H), 3.85 (s, 3H), 3.58 (s, 2H).

### Intermediate L: 6-Chloro-5-[5-(2-hydroxyphenyl)pyrimidin-2-yl]-1,3-dihydro-2H-indol-2-one

To a solution of intermediate 38 (0.16 g, 0.5 mmol) in DCM 10 mL), BBr₃ (2.08 g, 8.3 mmol) was added at 0° C. and stirred at room temperature for 2 h. The reaction mixture was quenched with sodium bicarbonate solution and extracted with ethyl acetate, and was washed with water and brine. The organic layer was dried over sodium sulphate and concentrated to yield the title product (0.1 g, 59.0%) as a pale brown solid. LCMS: (M+H)⁺=338.0; ¹H NMR: (DMSO-d₆, 300 MHz) δ 10.68 (s, 1H), 10.05 (s, 1H), 9.09 (s, 2H), 7.68 (s, 1H), 7.47-7.50 (d, 1H), 7.27-7.31 (t, 1H), 6.99-7.04 (m, 3H), 3.58 (s, 2H).

### Intermediate 39: Methyl 2-(4-formylphenyl)-2-methylpropanoate

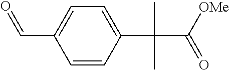

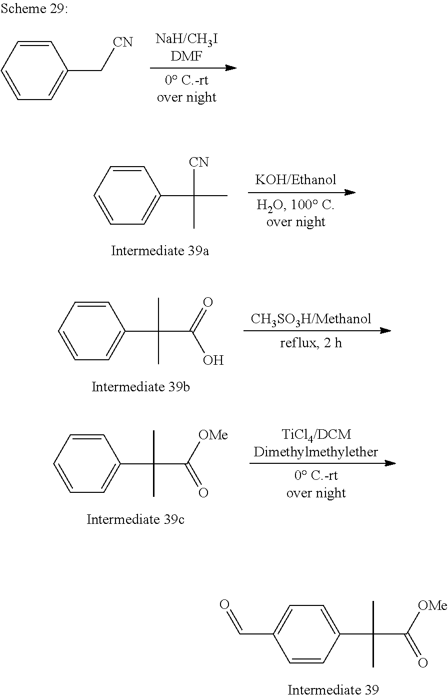

### Intermediate 39a: 2-Methyl-2-phenylpropanenitrile

To a stirred solution of phenylacetonitrile (5.0 g, 42.7 mmol) in DMF (30 mL) at 0 deg, sodium hydride (2.56 g, 2.5 eq) was added portion wise and stirred for 30 min. Then, methyl iodide (5.85 mL, 2.2 eq) was added and stirred for 12 h. The reaction mixture was quenched with ice water and extracted with ethyl acetate washed with water and brine solution, dried over sodium sulphate and concentrated. The obtained crude was purified by combi-flash to yield the title product (2.0 g, 33.32%) as a colourless liquid. ¹H NMR: (CDCl₃, 300 MHz) δ 7.40-7.42 (d 2H), 7.30-7.35 (t, 2H), 7.19-7.26 (m, 1H), 1.66 (s, 6H).

### Intermediate 39b: 2-Methyl-2-phenylpropanoic acid

To a solution of Intermediate 39a (2.0 g, 13.7 mmol) in ethanol: water (10 mL: 1 mL), KOH (4.63 g, 6.0 eq) was added and heated at 110° C. over night. After cooling, the reaction mixture was acidified with 1N HCl (PH=2) and extracted with ethyl acetate, washed with water, dried and concentrated to yield the title compound (1.6 g) as a white solid. LCMS: (M−H)⁺=163.0; ¹H NMR: (DMSO-d₆, 300 MHz) δ 12.31 (s, 1H), 7.33-7.34 (m, 4H), 7.22-7.27 (m, 1H), 1.47 (s, 6H).

### Intermediate 39c: Methyl 2-methyl-2-phenylpropanoate

A 100 mL sealed tube was charged with intermediate 39b (1.6 g, 9.7 mmol) and methanol (10 mL). To the above stirred solution CH₃SO₃H (1.6 mL) was added drop wise and refluxed for 2 h. The reaction mixture concentrated and was diluted with ice-water and neutralized with NaHCO₃, extracted with ethyl acetate, washed with water and dried. The product was obtained by concentrating under vacuo, which was further purified by combiflash to yield the title compound (1.0 g) as a colourless liquid. ¹H NMR: (CDCl₃, 300 MHz) δ 7.25-7.27 (d, 4H) 7.17-7.20 (m, 1H), 3.58 (s, 3H), 1.51 (s, 6H).

### Intermediate 39: Methyl 2-(4-formylphenyl)-2-methylpropanoate

To a solution of intermediate 39c (1.0 g, 1.0 eq) in DCM (30 mL) at 0° C. TiCl₄ (5.3 g, 5.0 eq) was added and stirred a room temperature for 30 min. Then, dichloromethylmethyl ether (3.22 g, 5.0 eq) was added under cooling and stirred at room temperature over night. The reaction mixture was quenched with 1N HCl (PH=3) and extracted with ethyl acetate, washed with water and dried. The product was obtained by concentrating under vacuo. The product was purified by combiflash to yield the title compound (0.1 g) as a colourless liquid. ¹H NMR: (CDCl₃, 300 MHz) δ 9.93 (d, 4H) 7.77-7.80 (d, 2H), 7.42-7.45 (d, 1H), 3.60 (s, 3H), 1.55 (s, 6H).

### Intermediate 40: 5-Formyl-2-methoxybenzoic acid

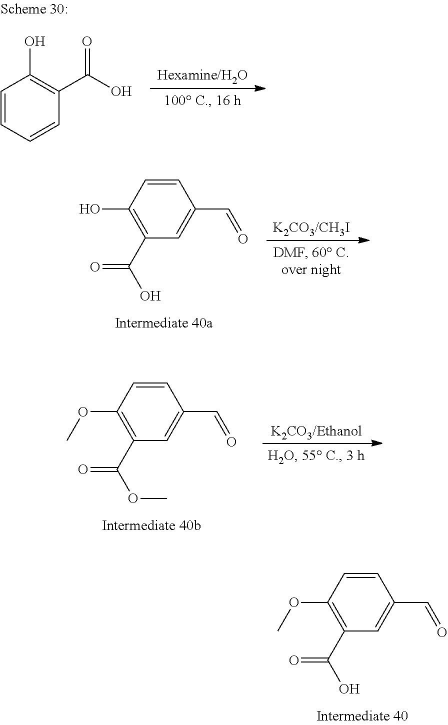

### Intermediate 40a: 5-formyl-2-hydroxybenzoic acid

To a solution of 2-hydroxybenzoic acid (2.0 g, 14.5 mmol) in water hexamethylenetetramine (4.05 g, 28.9 mmol) was added and refluxed at 100° C. for 16 h. After cooling, the reaction mixture was acidified with 1N HCl (PH=3) and extracted with ethyl acetate, washed with water and dried. The product was obtained by concentrating under vacuo to yield the title compound (2.2 g,) as a yellow solid. LCMS: (M−H)⁺=165.1

### Intermediate 40b: Methyl 5-formyl-2-methoxybenzoate

A 100 mL sealed tube was charged with 5-formyl-2-hydroxybenzoic acid (2.2 g, 13.2 mmol) and DMF (20 mL). To the above stirred solution K₂CO₃ (5.4 g, 39.7 mmol) and CH₃I (5.63 g, 39.7 mmol) was added and heated at 60° C. over night. The reaction mixture was diluted with water and extracted with ethyl acetate, washed with water and dried. The product was obtained by concentrating under vacuo. The product was purified by combiflash to yield the title compound (0.160 g, 6.24%) as a pale yellow solid. LCMS: (M+H)⁺=195.1; ¹H NMR: (DMSO-d₆, 300 MHz) δ 9.92 (s, 1H), 8.20-8.21 (d, 1H), 8.08-8.11 (dd, 1H), 7.37-7.39 (d, 1H), 3.94 (s, 3H), 3.83 (s, 3H).

### Intermediate 40: 5-Formyl-2-methoxybenzoic acid

To a solution of methyl 5-formyl-2-methoxybenzoate (0.120 g, 0.61 mmol) in ethanol: water (3:1), K₂CO₃ (0.170 g, 1.2 mmol) was added and stirred at room temperature over night. Then it was refluxed at 55° C. for 3 h. After cooling, the reaction mixture was acidified with 1N HCl (PH=3) and extracted with ethyl acetate, washed with water, dried and concentrated. The product was purified by column chromatography to yield the title compound (0.070 g, 64.76%) as a white solid. LCMS: (M+H)⁺=181.1; ¹H NMR: (DMSO-d₆, 300 MHz) δ 9.89 (s, 1H), 8.04-8.05 (d, 1H), 7.95-7.98 (d, 1H), 7.26-7.28 (d, 1H), 3.90 (s, 3H).

TABLE 4
Examples 31 to 46 were prepared by methods analogous to that described for Example 8.

| SI. No. | Structure | From | Physical Data |
|---|---|---|---|
| 31 | 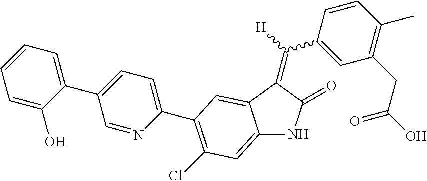 | H and 5 | LCMS: 497.0 (M + H)⁺ HPLC Rt: 13.11 and 13.28 min (Mixture) |
| 32 | 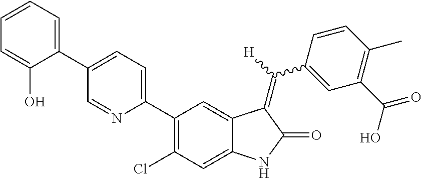 | H and 4 | LCMS: 483.0 (M + H)⁺ HPLC Rt: 12.67 and 13.46 min (Mixture) |
| 33 | 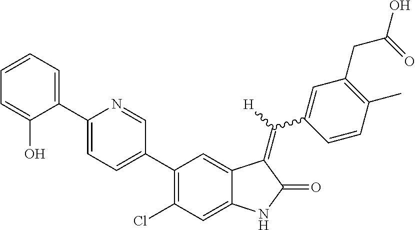 | I and 5 | LCMS: 497.1 (M + H)⁺ HPLC Rt: 15.16 and 15.78 min (Mixture) |
| 34 | 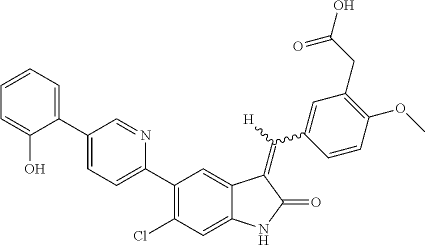 | H and 8 | LCMS: 513.0 (M + H)⁺ HPLC Rt: 12.46 and 12.87 min (Mixture) |
| 35 | 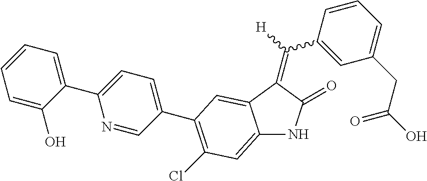 | I and 7 | LCMS: 483.0 (M + H)⁺ HPLC Rt: 16.28 and 17.38 min (Mixture) |
| 36 | 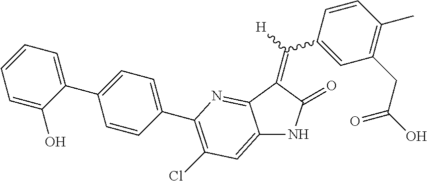 | G and 5 | LCMS: 497.1 (M + H)⁺ HPLC Rt: 15.72 and 16.13 min (Mixture) |
| 37 | 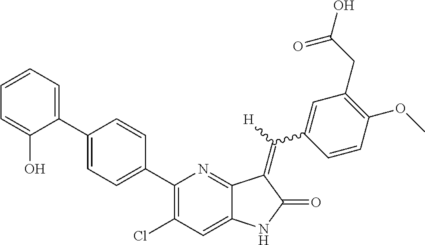 | G and 8 | LCMS: 513.1 (M + H)⁺ HPLC Rt: 15.29 and 15.74 min (Mixture) |
| 38 | 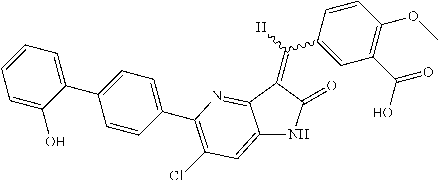 | G and 40 | LCMS: 497.0 (M − H)⁺ HPLC Rt: 14.77 and 15.13 min (Mixture) |
| 39 | 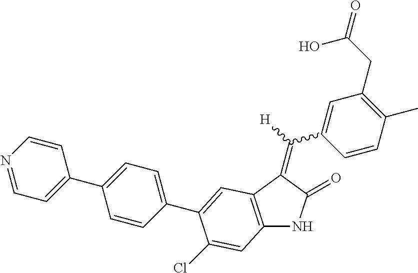 | J and 5 | LCMS: 481.1 (M + H)⁺ HPLC Rt: 10.23 min |
| 40 | 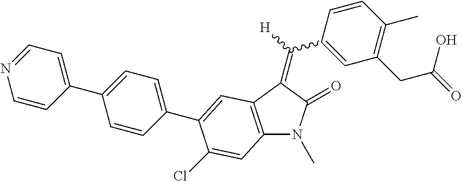 | J and 5 | LCMS: 493.1 (M − H)⁺ HPLC Rt: 11.46 and 12.50 min (Mixture) |
| 41 | 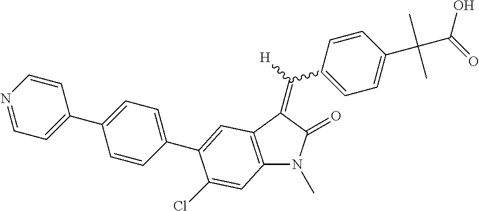 | J and 39 | LCMS: 509.1 (M + H)⁺ HPLC Rt: 11.46 and 12.50 min (Mixture) |
| 42 | 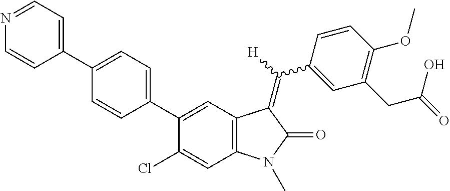 | J and 8 | LCMS: 509.4 (M − H)⁺ HPLC Rt: 11.25 and 12.35 min (Mixture) |
| 43 | 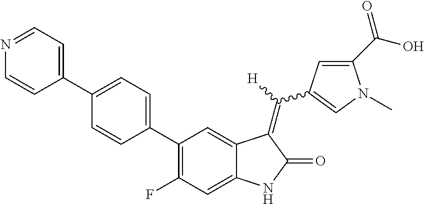 | K and 11 | LCMS: 440.1 (M + H)⁺ HPLC Rt: 8.95 and 9.69 min (Mixture) |
| 44 | 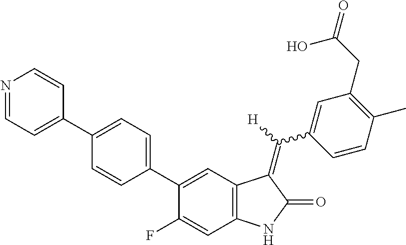 | K and 5 | LCMS: 463.0 (M − H)⁺ HPLC Rt: 9.87 and 10.70 min (Mixture) |
| 45 | 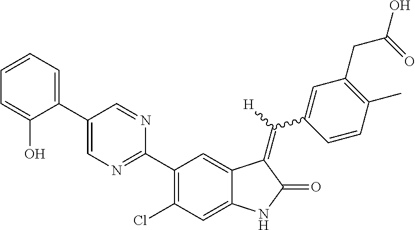 | L and 5 | LCMS: 498.0 (M + H)⁺ HPLC Rt: 13.22 and 13.78 min (Mixture); ¹H NMR (mixture of E/Z, DMSO, 300 MHz): δ 10.65 (s, 1H ), 9.06 (s, 2H), 8.30 (s, 1H), 7.65-7.77 (m, 2H), 7.42-7.57 (m, 2H), 7.25- 7.32 (m, 2H), 6.93-7.03 (m, 4H), 3.51-3.66 (m, 2H), 2.28- 2.31 (m, 3H). |
| 46 | 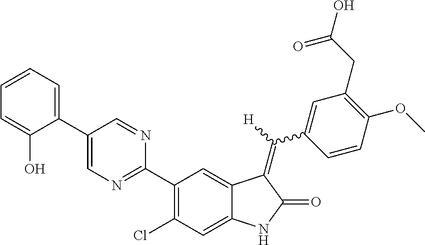 | L and 8 | LCMS: 514.0 (M + H)⁺ HPLC Rt: 12.73 and 13.40 min (Mixture); ¹H NMR (mixture of E/Z, DMSO, 300 MHz): δ 10.56 (m, 1H), 9.06-9.13 (m, 2H), 8.35 (s, 1H), 7.9-8.15 (m, 1H), 7.66-7.71 (m, 2H), 7.45- 7.51 (m, 1H), 7.25-7.30 (m, 1H), 7.01-7.13 (m, 1H), 6.92- 6.98 (m, 4H), 3.82-3.86 (m, 3H), 3.51 (m, 2H) |

### Intermediate O: 6-Chloro-5-[4′-(methylsulfonyl)biphenyl-4-yl]-1,3-dihydro-2H-indol-2-one

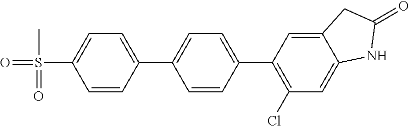 [SMILES: CS(=O)(=O)c1ccc(-c2ccc(-c3cc4c(cc3Cl)NC(=O)C4)cc2)cc1]

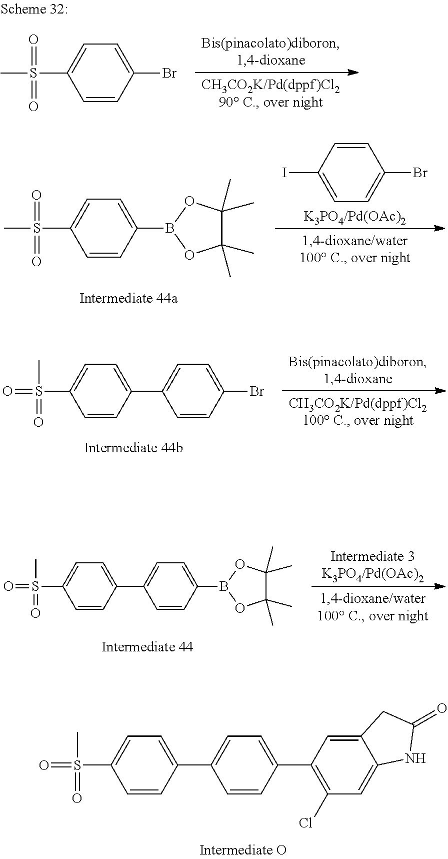

### Intermediate 44a: 4,4,5,5-Tetramethyl-2-[4-(methylsulfonyl)phenyl]-1,3,2-dioxaborolane

This intermediate was synthesized from 1-bromo-4-(methylsulfonyl)benzene (1.35 g, 1.0 eq) by following the similar procedure described for the intermediate 5a to yield the desired compound (1.2 g, 61.04%) as a white colour solid. ¹H NMR: (CDCl₃, 300 MHz) δ 7.87-7.91 (m, 4H), 2.98 (s, 3H), 1.29 (s, 12H).

### Intermediate 44b: 4′-Bromobiphenyl-4-yl methyl sulfone

To a solution of 1-bromo-4-iodobenzene (1.1 g, 1.0 eq) and intermediate 44a (1.2 g, 1.1 eq), potassium phosphate (2.47 g, 3.0 eq) and Pd(OAc)₂ (0.043 g, 0.05 eq) were taken into seal tube, degasified with nitrogen and heated at 100° C. for over night. After cooling, the reaction mass partitioned between water and ethyl acetate. The combined organic layer was dried over anhydrous Na₂SO₄ and concentrated. The product was purified by combiflash to yield the title product (0.7 g, 57.85%) as light pink solid. ¹H NMR: (CDCl₃, 300 MHz) δ 7.92-7.98 (d, 2H), 7.64-7.71 (d, 2H), 7.53-7.58 (d, 2H), 7.36-7.40 (d, 2H), 2.99 (s, 3H).

### Intermediate 44: 4,4,5,5-Tetramethyl-2-[4′-(methylsulfonyl)biphenyl-4-yl]-1,3,2-dioxaborolane

This intermediate was synthesized form intermediate 4b by following the similar procedure described for the intermediate 5a to yield the desired compound (0.45 g, 66.20%) as an off white solid. LCMS: (M+H)⁺=359.3; ¹H NMR: (CDCl₃, 300 MHz) δ 7.93-7.96 (m, 2H), 7.84-7.87 (m, 2H), 7.71-7.74 (m, 2H), 7.54-7.57 (m, 2H), 3.03 (s, 3H), 1.30 (s, 12H).

### Intermediate O: 6-Chloro-5-[4′-(methylsulfonyl)biphenyl-4-yl]-1,3-dihydro-2H-indol-2-one

This intermediate was synthesized form intermediate 44 and Intermediate 3 by following the similar procedure described for the intermediate 44b to yield the desired compound (0.3 g, 66.70%) as an off white solid. LCMS: (M−H)⁺=396.1

### Intermediate P: 6-Chloro-5-[4′-(1H-pyrazol-1-yl)biphenyl-4-yl]-1,3-dihydro-2H-indol-2-one

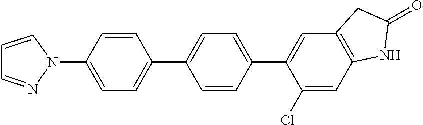 [SMILES: O=C1Cc2cc(-c3ccc(-c4ccc(-n5cccn5)cc4)cc3)c(Cl)cc2N1]

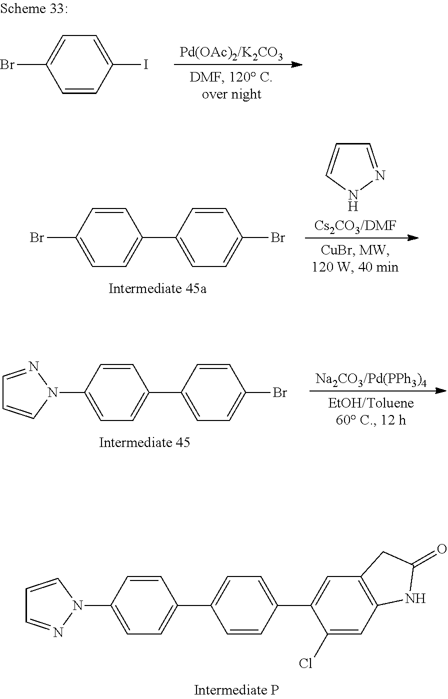 [SMILES: Brc1ccc(I)cc1 ; c1cn[nH]c1 ; Brc1ccc(-c2ccc(Br)cc2)cc1 ; Brc1ccc(-c2ccc(-n3cccn3)cc2)cc1 ; O=C1Cc2cc(-c3ccc(-c4ccc(-n5cccn5)cc4)cc3)c(Cl)cc2N1]

### Intermediate 45a: 4, 4′-Dibromobiphenyl

To a stirred solution of compound 1-bromo-4-iodobenzene (10.0 g, 35.3 mmol) in DMF (80.0 mL) potassium carbonate (58.5 g, 12.0 eq) and Pd(OAc)₂ (0.396 g, 0.05 eq) was added under argon atmosphere. The reaction mixture was heated at 120° C. over night. After cooling, the reaction mixture was quenched with ice and extracted with ethyl acetate. The organic layer was washed with water and brine, dried over Na₂SO₄ and concentrated under reduced pressure. The product was purified by column chromatography to yield the title compound (0.75 g, 6.81%) as a white solid.

### Intermediate 45: 1-(4′-Bromobiphenyl-4-yl)-1H-pyrazole

A micro wave vessel was charged with intermediate 45a (0.1 g, 0.324 mmol), pyrazole (0.01 g, 0.5 eq) and DMF (2.0 mL). To the stirred solution Cs₂CO₃ (0.21 g, 2.0 eq) was added and degassed with argon gas for 15 min. Then copper bromide (1.0 mg, 0.02 eq) was added and irradiate at 120 w for 40 min. After cooling, the reaction mixture was quenched with ice and extracted with ethyl acetate. The organic layer was washed with water and brine, dried over Na₂SO₄ and concentrated under reduced pressure to yield the title compound (0.05 g, 52.19%) as a white solid. LCMS: (M−2)⁺=301.1

### Intermediate P: 6-Chloro-5-[4′-(1H-pyrazol-1-yl)biphenyl-4-yl]-1,3-dihydro-2H-indol-2-one

This intermediate was synthesized from intermediate 45 and intermediate 34 by following the similar procedure described for the intermediate A to yield the desired compound (0.3 g, crude product) as a brown solid. LCMS: (M+H)⁺=386.2. Similar methodology has been utilized to prepare analogues of Intermediate 0.

### Intermediate P2: 6-fluoro-5-[4′-(1H-pyrazol-1-yl) biphenyl-4-yl]-1,3-dihydro-2H-indol-2-one

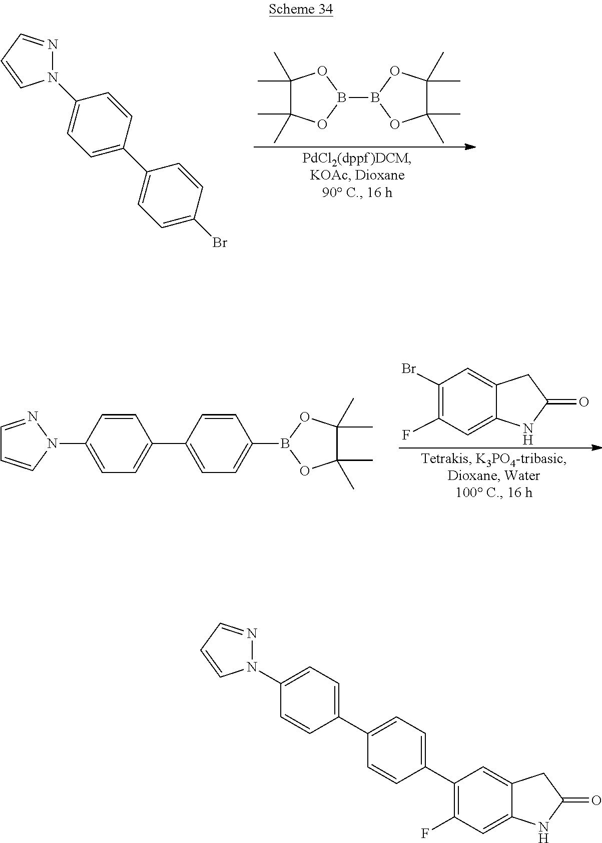

### Step-1: 1-(4-(4,4,5,5-tetramethyl-1,3-dioxoborolan-2-yl)biphenyl-4-yl)-1H-pyrazole

To a stirred solution of compound 1-(4′-bromobiphenyl-4-yl)-1H-pyrazole (25 g, 0.0833 mol) in 1,4-dioxane (250 ml), Bis(pinacalato)diboron (25.3 g, 1.2 eq) and KOAC (24.5 g, 3.0 eq) was added and degassed with nitrogen gas for 15 min. Then PdCl₂ (dppf) DCM complex (3.4 g, 0.05 eq) was added to it. The reaction mixture was heated at 90° C. for 16 hours. The reaction mixture was filtered through celite and washed with ethyl acetate. The organic layer was washed with water and brine, dried over Na₂SO₄ and concentrated under reduced pressure and purified by column-chromatography to yield the title compound (23 g, 79%) as a white solid. LCMS: (M+H)⁺=347.0; ¹H NMR: (DMSO-d6, 300 MHz) δ 8.589-8.581 (d, 1H), 7.976-7.947 (d, 2H), 7.853-7.824 (d, 2H), 7.784-7.768 (m, 4H), 7.76-7.733 (d, 1H), 6.585-6.572 (t, 1H), 1.319 (s, 12H).

### Step-2: 6-fluoro-5-[4′-(1H-pyrazol-1-yl)biphenyl-4-yl]-1,3-dihydro-2H-indol-2-one, Intermediate P2

To a stirred solution of compound 1-(4-(4,4,5,5-tetramethyl-1,3-dioxoborolan-2-yl) biphenyl-4-yl)-1H-Pyrazole. (0.842 g, 0.0024 mol) in 1, 4-dioxane water (12.0 ml), 5-bromo-6-fluoro-1,3-dihydro-2H-indol-2-one (0.4 g, 1.0 eq) and K₃PO₄ tribasic (1.08 g, 3.0 eq) was added and degassed with nitrogen gas for 15 min. Then Pd (PPh₃)₄ (0.98 g, 0.05 eq) was added. The reaction mixture was heated at 100° C. for 16 hours. The reaction mixture was diluted with water and ethyl acetate to get solid. The solid was filtered and again washed with water and dried to get pure off white solid (0.5 g, 55.7%). LCMS: (M+H)⁺=370; ¹H NMR: (DMSO-d6, 300 MHz) δ 10.61 (s, 1H), 8.58-8.59 (d, 1H), 7.95-7.98 (d, 2H), 7.79-7.88 (m, 4H), 7.59-7.62 (d, 2H), 7.40-7.43 (d, 1H), 6.74-6.78 (d, 1H), 6.58-6.59 (d, 1H), 3.53 (s, 2H).

### Intermediate P3: 6-fluoro-5-[4′-(1H-1,2,4-triazol-1-yl)biphenyl-4-yl]-1,3-dihydro-2H-indol-2-one

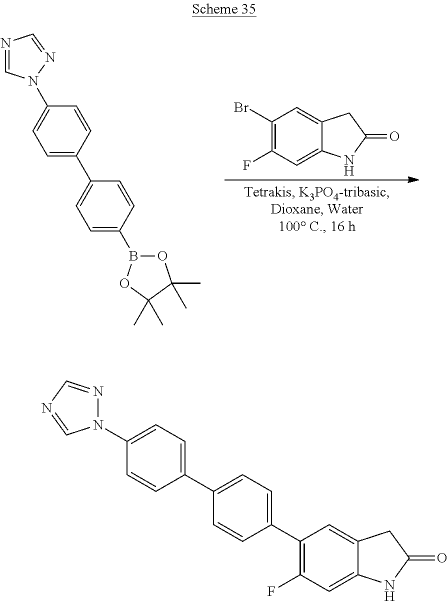 [SMILES: CC1(C)OB(c2ccc(-c3ccc(-n4cncn4)cc3)cc2)OC1(C)C ; O=C1Cc2cc(Br)c(F)cc2N1 ; O=C1Cc2cc(-c3ccc(-c4ccc(-n5cncn5)cc4)cc3)c(F)cc2N1]

### Step-1: 6-fluoro-5-[4′-(1H-1,2,4-triazol-1-yl)biphenyl-4-yl]-1,3-dihydro-2H-indol-2-one, Intermediate P3

A sealed-tube was charged with compound 5-bromo-6-fluoro-1,3-dihydro-2H-indol-2-one (1.0 g, 0.00434 mol) and 1-(4-(4,4,5,5-tetramethyl-1,3-dioxoborolan-2-yl) biphenyl-4-yl)-1H-triazole (2.1 g, 1.4 eq) in Dioxane water (25.0 ml) and K₃PO₄ tribasic (2.7 g, 3.0 eq) and degassed with nitrogen gas for 15 min. Then was added Pd(PPh₃)₄ (0.25 g, 0.05 eq). The reaction mixture was heated at 100° C. 16 hours. The reaction mixture was diluted with water and ethyl acetate to get solid. The solid was filtered and again washed with water and dried to get pure off white solid (1.0 g, 62.5%). LCMS: (M+H)⁺=371; ¹H NMR: (DMSO-d6, 300 MHz) δ 10.62 (s, 1H), 9.38 (s 1H), 8.28 (s, 1H), 7.92-8.0 (m, 4H), 7.83-7.85 (d, 2H), 7.61-7.63 (d, 2H), 7.41-7.44 (d, 1H), 6.75-6.79 (d, 1H), 3.54 (s, 2H).

### Intermediate P4: 6-chloro-5-[4′-(1H-1,2,4-triazol-1-yl)biphenyl-4-yl]-1,3-dihydro-2H-pyrrolo[3,2-b]pyridin-2-one

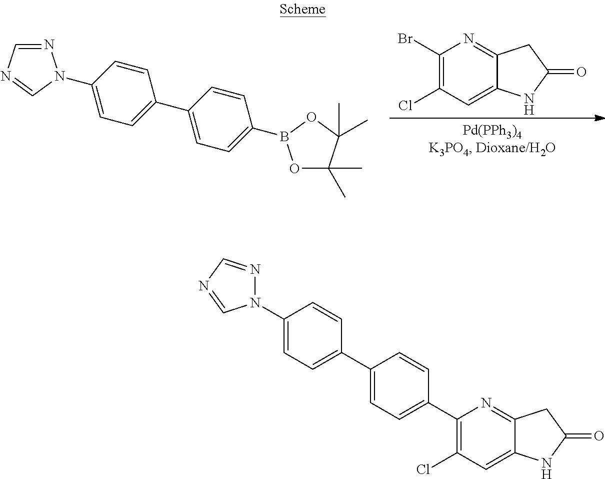

### Step-1: 6-chloro-5-[4′-(1H-1,2,4-triazol-1-yl)biphenyl-4-yl]-1,3-dihydro-2H-pyrrolo[3,2-b]pyridin-2-one, Intermediate P4

To a stirred solution of compound 5-bromo-6-chloro-1,3-dihydro-2H-pyrrolo[3,2-b]pyridin-2-one (0.300 g, 1 eq) in 1,4-dioxane (15 mL) and water (3 mL), 1-(4-(4,4,5,5-tetramethyl-1,3-dioxoborolan-2-yl)biphenyl-4-yl)-1,2,4-triazole (0.421 g, 1 eq) and potassium phosphate tribasic (0.769 g, 3 eq) was added. This reaction mixture was degassed with Nitrogen for 15 min followed by the addition of Pd(PPh3)4 (0.069 g, 0.05 eq). It was heated to 100° C. for 16 h. The reaction mixture was cooled to room temperature and poured in to ice water and extracted with Ethyl acetate (20×3 ml). organic layer was washed with water and brine solution. The organic phase was dried over anhydrous Na₂SO₄ and then centrated to afford the crude product was washed with Diethyl ether/Ethyl acetate/Hexane (5:2:3) to yield the title compound (0.350 g, yield: 74%) as a Brown color solid. LCMS: (M+H)⁺=388, ¹H NMR: (DMSO-d6, 300 MHz) δ 10.79 (s, 1H), 9.38 (s, 1H), 8.28 (s, 1H), 7.93-8.01 (m, 4H), 7.84 (d, 2H), 7.74 (d, 2H), 7.33 (s, 1H), 3.68 (s, 2H).

### Intermediate P5: 6-chloro-5-[4′-(1H-1,2,3-triazol-1-yl)biphenyl-4-yl]-1,3-dihydro-2H-indol-2-one

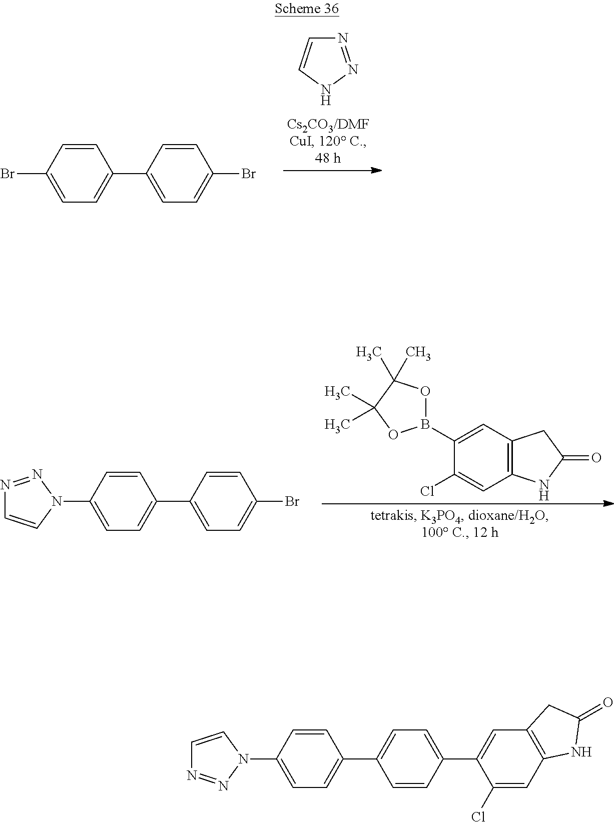 [SMILES: c1c[nH]nn1 ; Brc1ccc(-c2ccc(Br)cc2)cc1 ; Brc1ccc(-c2ccc(-n3ccnn3)cc2)cc1 ; CC1(C)OB(c2cc3c(cc2Cl)NC(=O)C3)OC1(C)C ; O=C1Cc2cc(-c3ccc(-c4ccc(-n5ccnn5)cc4)cc3)c(Cl)cc2N1]

### Step-1: 1-(4′-bromobiphenyl-4-yl)-1H-1,2,3-triazole

To a stirred solution of compound 4,4′-dibromobiphenyl (20.0 g, 64.1 mmol) in DMF (200.0 mL) 1,2,3-triazole (5.9 mL, 102.5 mmol), potassium carbonate (17.6 g, 128.2 mmol) and CuI (1.2 g, 6.41 mmol) was added under nitrogen atmosphere. The reaction mixture was degassed for 15 min and then heated at 120° C. for 48 h. After cooling, the reaction mixture was quenched with water and extracted with ethyl acetate. The organic layer was washed with water and brine, dried over Na₂SO₄ and concentrated under reduced pressure. The crude product was purified by column chromatography to yield the title compound (3 g, 15%) as an off white solid. LCMS: (M+2)⁺=302. ¹H NMR: (DMSO-d6, 300 MHz) δ 10.60 (s, 1H), 8.92 (s, 1H), 7.96-8.06 (m, 4H), 7.83-7.86 (d, 2H), 7.73-7.76 (d, 1H), 7.51-7.54 (d, 2H), 7.31 (s, 1H), 6.97 (s, 1H), 3.54 (s, 2H).

### Step-2: 6-chloro-5-[4′-(1H-1,2,3-triazol-1-yl)biphenyl-4-yl]-1,3-dihydro-2H-indol-2-one, Intermediate P5

To a stirred solution of compound 1-(4′-bromobiphenyl-4-yl)-1H-1,2,3-triazole (0.08 g, 0.23 mmol) in 1,4-dioxane (5 mL) and water (1 mL), 6-chloro-5-(4,4,5,5-tetramethyl-1,3,2-dioxaborolan-2-yl)-1,3-dihydro-2H-indol-2-one (0.067 g, 0.23 mmol) and potassium phosphate tribasic (0.146 g, 0.69 mmol) was added. This reaction mixture was degassed with Nitrogen for 15 min followed by the addition of Pd(PPh₃)₄ (0.013 g, 0.011 mmol). It was heated to 100° C. for 16 h. After cooling, the reaction mixture was diluted with water and filtered through Buchner funnel. Solid was thoroughly washed with excess water and then with EtOAC (5 mL). It was dried well under vacuum to afford title compound (0.05 g, yield: 56%) as a pale brown solid.

### Intermediate P6: 6-chloro-5-[2-fluoro-4′-(1H-1,2,4-triazol-1-yl)biphenyl-4-yl]-1,3-dihydro-2H-indol-2-one

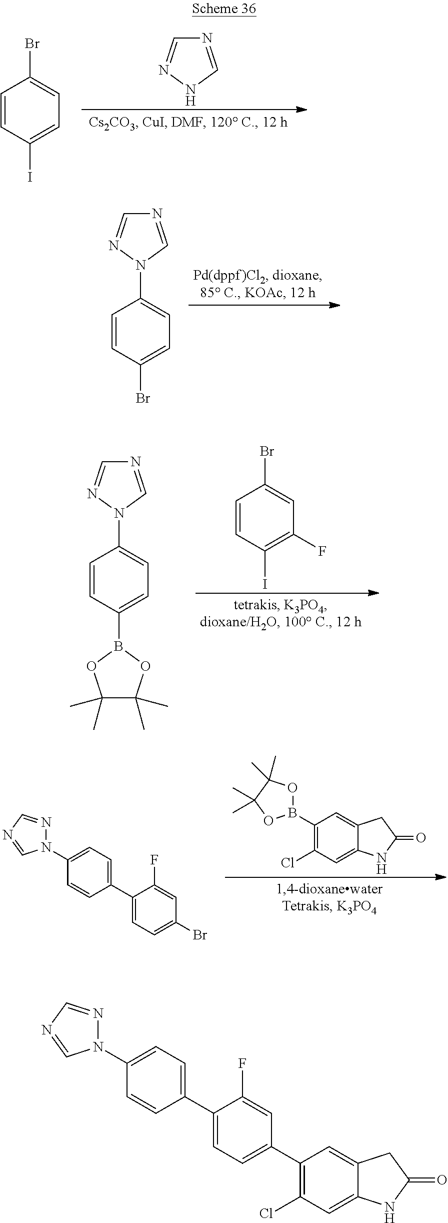

### Step-1: 1-(4-bromophenyl)-1H-1, 2, 4-triazole

To a stirred solution of 1-bromo-4-iodobenzene (2.0 g, 7.07 mmol), 1H-1,2,4-triazole (0.390 g, 5.66 mmol) in DMF (20.0 mL), Cs₂CO₃ (6.9 g, 21.21 mmol) was added and degassed with argon gas for 15 min. Then copper Iodide (0.404 g, 2.12 mmol) was added and heated at 120° C. for 12 h. After cooling, the reaction mixture was quenched with ice and extracted with ethyl acetate. The organic layer was washed with water and brine, dried over Na₂SO₄ and concentrated under reduced pressure. The product was purified by column chromatography to yield the title compound (1.2 g, 75.75%) as a pale brown solid. LCMS: (M+H)⁺=224-226.0. ¹H NMR: (DMSO-d6, 300 MHz) δ 9.33 (s, 1H), 8.26 (s, 1H), 7.85 (d, 2H), 7.77 (d, 2H).

### Step-2: 1-(4-(4,4,5,5-tetramethyl-1,3,2-dioxoboralon-2-yl)phenyl)-1H-1,2,4-triazole

To a stirred solution of compound 1-(4-bromophenyl)-1H-1,2,4-triazole (1.0 g, 4.46 mmol) in 1,4-dioxane (10.0 mL), potassium acetate (0.880 g, 8.92 mmol) and Bis(pinacolato)diboron (1.36 g, 5.36 mmol) was added under argon atmosphere. After 15 min Pd(dppf)Cl₂ DCM complex (0.072 g, 0.089 mmol) was added to the reaction mixture under Argon and again degassed for 5 min. The reaction mixture was heated at 85° C. 12 h. After cooling, the reaction mixture was quenched with ice and extracted with ethyl acetate. The organic layer was washed with water and brine, dried over Na₂SO₄ and concentrated under reduced pressure. The product was purified by column chromatography to yield the title compound (1.2 g, 99.16%) as a pale orange solid. LCMS: (M+H)⁺=272.0. ¹H NMR: (DMSO-d6, 300 MHz) δ 9.39 (s, 1H), 8.27 (s, 1H), 7.92 (d, 2H), 7.84 (d, 2H), 1.32 (s, 12H).

### Step-3 1-(4′-bromo-2′-fluorobiphenyl-4-yl)-1H-1, 2, 4-triazole

To a stirred solution of compound 1-(4-(4,4,5,5-tetramethyl-1,3,2-dioxoboralon-2-yl)phenyl)-1H-1,2,4-triazole (0.8 g, 2.95 mmol) in 1,4-dioxane/Water (10.0/3 mL), 4-bromo-2-fluoro-1-iodobenzene (0.887 g, 2.95 mmol) and K₃PO₄ tribasic (1.88 g, 8.85 mmol) was added and degassed with argon atmosphere. After 15 min Tetrakis (0.170 g, 0.148 mmol) was added to the reaction mixture under argon and again degassed for 5 min. The reaction mixtures was heated at 100° C. 12 h. After cooling, the reaction mixture was quenched with ice and extracted with ethyl acetate. The organic layer was washed with water and brine, dried over Na₂SO₄ and concentrated under reduced pressure, to yield the title compound (0.76 g, 80.96%) as a pale yellow solid. LCMS: (M−H)⁺=320.0 ¹H NMR: (DMSO-d6, 300 MHz) δ 9.38 (s, 1H), 8.28 (s, 1H), 7.90 (d, 2H), 7.76 (d, 2H), 7.69 (s, 1H) 7.57 (d, 2H).

### Step-4: 6-chloro-5-[2-fluoro-4′-(1H-1,2,4-triazol-1-yl)biphenyl-4-yl]-1,3-dihydro-2H-indol-2-one, Intermediate P6

To a stirred solution of compound 1-(4′-bromo-2′-fluorobiphenyl-4-yl)-1H-1,2,4-triazole (0.5 g, 1.57 mmol) 6-chloro-5-(4,4,5,5-tetramethyl-1,3,2-dioxaborolan-2-yl)-1,3-dihydro-2H-indol-2-one (0.46 g, 1.57 mmol) in 1,4-dioxane/Water (6.0/2.0 mL), K₃PO₄ tribasic (1.0 g, 4.71 mmol) was added and degassed with argon atmosphere. After 15 min tetrakis (0.09 g, 0.78 mmol) was added to the reaction mixture under argon and again degassed for 5 min. The reaction mixtures was heated at 100° C. for 4 h. After cooling, the reaction mixture was quenched with ice and extracted with ethyl acetate. The organic layer was washed with water and brine, dried over Na₂SO₄ and concentrated under reduced pressure to yield the title compound (0.530 g, 83.30% as a pale brown solid. LCMS: (M−H)⁺=403.0. ¹H NMR: (DMSO-d6, 300 MHz) δ 10.65 (s, 1H), 9.40 (s, 1H), 8.29 (s, 1H), 8.0-8.03 (d, 2H), 7.8-7.83 (d, 2H), 7.66-7.71 (t, 1H), 7.34-7.41 (m, 3H) 6.98 (s, 1H), 3.55 (s, 2H).

### Intermediate P7: 6-fluoro-5-[4′-(1H-1,2,3-triazol-1-yl)biphenyl-4-yl]-1,3-dihydro-2H-indol-2-one

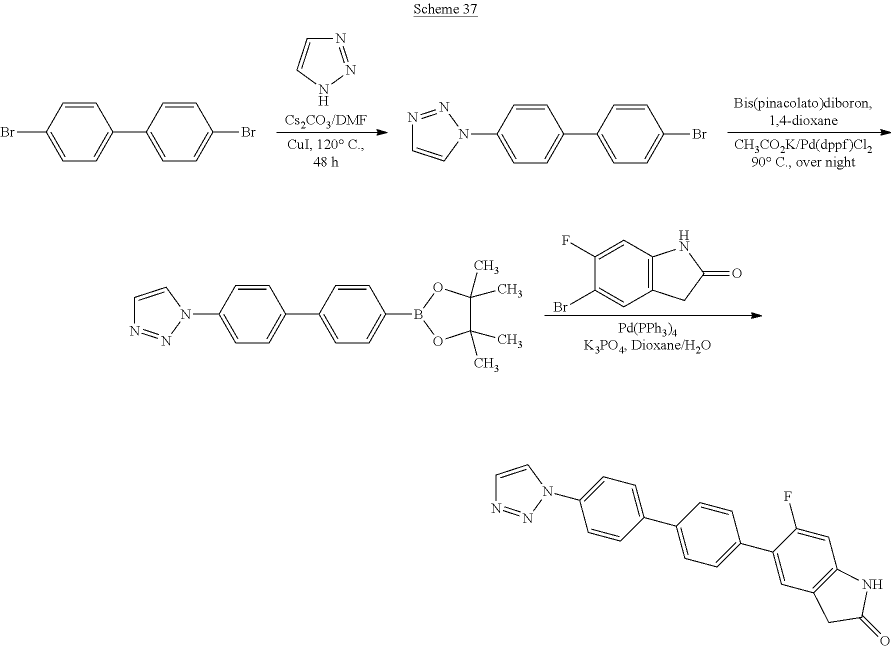 [SMILES: c1c[nH]nn1 ; Brc1ccc(-c2ccc(Br)cc2)cc1 ; Brc1ccc(-c2ccc(-n3ccnn3)cc2)cc1 ; CC1(C)OB(c2ccc(-c3ccc(-n4ccnn4)cc3)cc2)OC1(C)C ; O=C1Cc2cc(Br)c(F)cc2N1 ; O=C1Cc2cc(-c3ccc(-c4ccc(-n5ccnn5)cc4)cc3)c(F)cc2N1]

### Step-1: 1-(4′-bromobiphenyl-4-yl)-1H-1,2,3-triazole

To a stirred solution of compound 4,4′-dibromobiphenyl (20.0 g, 64.1 mmol) in DMF (200.0 mL), 1,2,3-triazole (5.9 mL, 102.5 mmol), potassium carbonate (17.6 g, 128.2 mmol) and CuI (1.2 g, 6.41 mmol) was added under nitrogen atmosphere. The reaction mixture was degassed for 15 min and then heated at 120° C. for 48 h. After cooling, the reaction mixture was quenched with water and extracted with ethyl acetate. The organic layer was washed with water and brine, dried over Na₂SO₄ and concentrated under reduced pressure. The crude product was purified by column chromatography to yield the title compound (3 g, 15%) as an off white solid. LCMS: (M+2)⁺=302

### Step-2: 1-[4′-(4,4,5,5-tetramethyl-1,3,2-dioxaborolan-2-yl)biphenyl-4-yl]-1H-1,2,3-triazole

To a stirred solution of compound 1-(4′-bromobiphenyl-4-yl)-1H-1,2,3-triazole (7.0 g, 23.3 mmol) in 1,4-dioxane, Bis(pinacolato)diborane (7.1 g, 28 mmol) and potassium acetate (6.8 g, 69.9 mmol) was added. This reaction mixture was degassed with Nitrogen for 15 min followed by the addition of PdCl2(dppf)DCM complex (0.96 g, 1.2 mmol). It was heated to 90° C. for 16 h. The reaction mixture was diluted with EtOAc (200 mL) and then filtered through celite pad. The filtrate was washed with water and then with saturated brine solution successively. The organic phase was dried over anhydrous Na₂SO₄ and then concentrated to afford the crude product which was purified by Column chromatography to yield the title compound (4.9 g, yield: 60%) as an off white solid. LCMS: (M+H)⁺=348.3

### Step-3: 6-fluoro-5-[4′-(1H-1,2,3-triazol-1-yl)biphenyl-4-yl]-1,3-dihydro-2H-indol-2-one

To a stirred solution of compound 1-(4′-bromobiphenyl-4-yl)-1H-1,2,3-triazole (0.7 g, 2.01 mmol) and 5-bromo-6-fluoro-1,3-dihydro-2H-indol-2-one (0.37 g, 1.61 mmol) in 1,4-dioxane (5 mL) and water (2 mL), potassium phosphate tribasic (1.28 g, 6.05 mmol) was added. This reaction mixture was degassed with Nitrogen for 15 min followed by the addition of Pd(PPh₃)₄ (0.11 g, 0.1 mmol). It was heated to 100° C. for 24 h. After cooling, the reaction mixture was diluted with water and the filtered through Buchner funnel. Solid thus obtained was thoroughly washed with excess water and then with EtOAC (50 mL). It was dried well under vacuum to afford title compound (0.7 g, yield: 33%) as a pale brown solid, Intermediate P7

### Intermediate P8: 6-chloro-5-[4′-(2-methyl-1H-imidazol-1-yl)biphenyl-4-yl]-1,3-dihydro-2H-indol-2-one

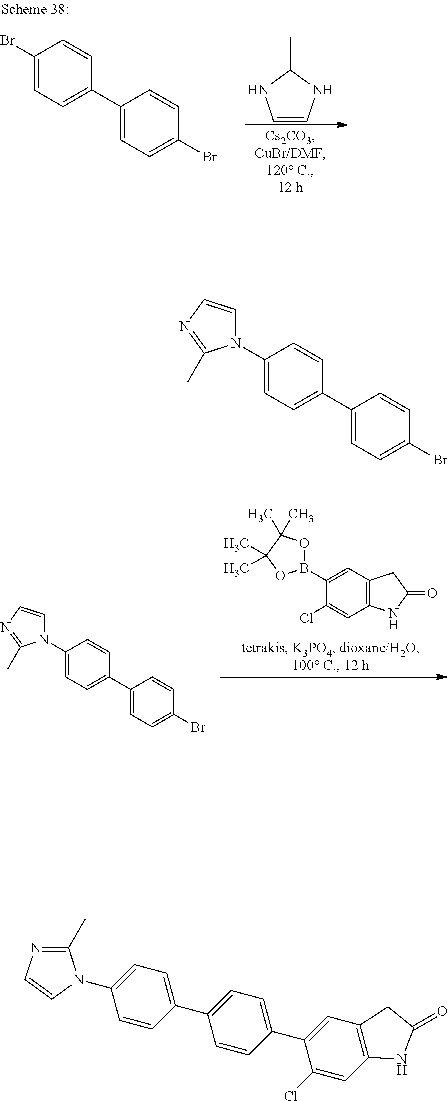

### Step-1: 1-(4′-bromobiphenyl-4-yl)-2-methyl-1H-imidazole

To a stirred solution of compound 4,4′-dibromobiphenyl (5.0 g, 16.02 mmol) in DMF (25 mL), 2-methyl-2,3-dihydro-1H-imidazole (1.31 g, 16.02 mmol) and cesium carbonate (10.4 g, 32.04mmo0l) was added. This reaction mixture was degassed with Nitrogen for 15 min followed by the addition of CuBr (0.183 g, 1.602 mmol). It was heated to 120° C. for 16 h. The reaction mixture was diluted with EtOAc (200 mL) and then filtered through celite pad. The filtrate was washed with water and then with saturated brine solution successively. The organic phase was dried over anhydrous Na₂SO₄ and then concentrated to afford the crude product which was purified by Column chromatography to yield the title compound (4.9 g, yield: 60%) as an off white solid. LCMS: (M+2)⁺=315.2

### Step-2: 6-chloro-5-[4′-(2-methyl-1H-imidazol-1-yl)biphenyl-4-yl]-1,3-dihydro-2H-indol-2-one, Intermediate P8

To a stirred solution of compound 1-(4′-bromobiphenyl-4-yl)-2-methyl-1H-imidazole (0.6 g, 1.92 mmol) in 1,4-dioxane (8 mL) and water (2 mL), 6-chloro-5-(4,4,5,5-tetramethyl-1,3,2-dioxaborolan-2-yl)-1,3-dihydro-2H-indol-2-one (0.67 g, 2.3 mmol) and potassium phosphate tribasic (1.22 g, 5.77 mmol) was added. This reaction mixture was degassed with Nitrogen for 15 min followed by the addition of Pd(PPh₃)₄ (0.11 g, 0.096 mmol). It was heated to 100° C. for 16 h. The reaction mixture was diluted with EtOAc (100 mL) and then filtered through celite pad. The filtrate was washed with water and then with saturated brine solution successively. The organic phase was dried over anhydrous Na₂SO₄ and then concentrated to afford the crude product which was purified by Column chromatography to yield Intermediate P8 (0.3 g, yield: 42%) as an off white solid. LCMS: (M−1)⁻=398.0

### Intermediate P9: 6-chloro-5-{4-[6-(1H-1,2,4-triazol-1-yl)pyridin-3-yl]phenyl}-1,3-dihydro-2H-indol-2-one

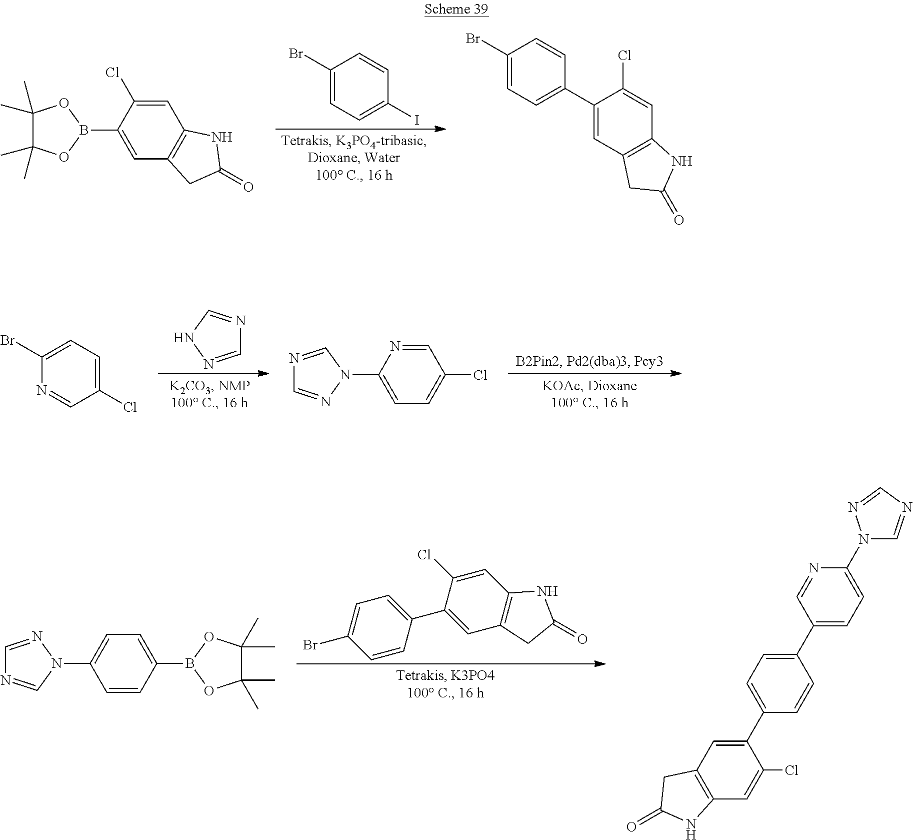

### Step-1: 5-(4-bromophenyl)-6-chloro-1,3-dihydro-2H-indol-2-one

To a stirred solution of compound 6-chloro-5-(4,4,5,5-tetramethyl-1,3,2-dioxaborolan-2-yl)-1,3 dihydro-2H-indol-2-one (1.75 g, 0.00596 mol) in 1,4-dioxane water (15.0 ml), 1-bromo-4-iodobenzene (1.68 g, 1.0 eq) and K₃PO₄ tribasic (3.8 g, 3.0 eq) was added and degassed with nitrogen gas for 15 min. Then was added Pd (PPh₃)₄ (0.34 g, 0.05 eq). The reaction mixture was heated at 100° C. for 16 hours. The reaction mixture was filtered through celite pad and washed with ethyl acetate. The organic layer was washed with water and brine, dried over Na₂SO₄ and concentrated under reduced pressure and purified by column-chromatography to yield the title compound (1.1 g, 57.2%) as a brown solid. LCMS: (M+H)⁺=323.9.

### Step-2: 5-chloro-2-(1H-1,2,4-triazol-1-yl)pyridine

To a stirred solution of compound 2-bromo-5-chloropyridine (1.0 g, 0.0052 mol) in NMP (12.0 mL), potassium carbonate (2.1 g, 3.0 eq) and 1H-1,2,4-triazole (0.431 g, 1.2 eq) was added. The reaction mixture was heated at 100° C. 16 hours. After cooling, the reaction mixture was quenched with water and extracted with Diethyl ether. The organic layer was, dried over Na₂SO₄ and concentrated under reduced pressure. The product was purified by column chromatography to yield the title compound (0.6 g, 63%) as a off white solid. ¹H NMR: (CDCl3, 300 MHz) δ 9.13 (s, 1H), 8.42 (s 1H), 8.1 (s, 1H), 7.87-7.86 (d, 2H).

### Step-3: 5-(4,4,5,5-tetramethyl-1,3,2-dioxaborolan-2-yl)-2-(1H-1,2,4-triazol-1-yl)pyridine

To a stirred solution of compound 5-chloro-2-(1H-1,2,4-triazol-1-yl)pyridine (0.32 g, 0.00175 mol) in 1,4-dioxane (8.0 ml), Bis(pinacolato)diboron (0.535 g, 1.2 eq) and KOAC (0.517 g, 3.0 eq) was added and degassed with argon gas for 15 min. Then tricyclohexylphosphine (0.009 g, 0.02 eq) and Pd₂(dba)₃ (0.08 g, 0.05 eq) was added. The reaction mixture was heated at 100° C. for 16 hours. The reaction mixture was filtered through celite and washed with ethyl acetate. The organic layer was washed with water and brine, dried over Na₂SO₄ and concentrated under reduced pressure and purified by column-chromatography to yield the title compound (0.105 g, 21%) as a light brown solid. LCMS: (M+H)⁺=273.3.

### Step-4: 6-chloro-5-{4-[6-(1H-1,2,4-triazol-1-yl)pyridin-3-yl]phenyl}-1,3-dihydro-2H-indol-2-one, Intermediate P9

To a stirred solution of compound 5-(4,4,5,5-tetramethyl-1,3,2-dioxaborolan-2-yl)-2-(1H-1,2,4-triazol-1-yl)pyridine (0.105 g, 0.384 mmol) in 1,4-dioxane water (15.0 ml) was added 5-(4-bromophenyl)-6-chloro-1,3-dihydro-2H-indol-2-one (0.124 g, 1.0 eq) and K₃PO₄ tribasic (0.244 g, 3.0 eq) and degassed with nitrogen gas for 15 min. Then Pd(PPh₃)₄ (0.022 g, 0.05 eq) was added. The reaction mixture was heated at 100° C. for 16 hours. The reaction mixture was filtered through celite pad and washed with ethyl acetate. The organic layer was washed with water and brine, dried over Na₂SO₄ and concentrated under reduced pressure and purified by column-chromatography to yield the title compound (0.07 g, 46%) as a light yellow solid. LCMS: (M+H)⁺=389.1. ¹H NMR: (DMSO-d6, 300 MHz) δ 10.6 (s, 1H), 9.44 (s, 1H), 8.94 (s, 1H), 8.467-8.461 (d, 2H), 8.3 (s, 1H), 7.99-7.96 (d, 1H), 7.9-7.87 (d, 2H), 7.56-7.53 (d, 2H), 7.3 (s, 1H), 6.96 (s, 1H), 3.56 (s, 2H).

| SI. No. | Structure | Intermediate |
|---|---|---|
| 1 | 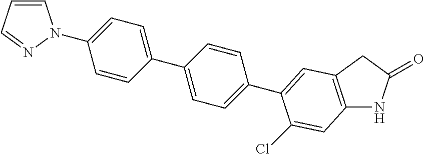 [SMILES: O=C1Cc2cc(-c3ccc(-c4ccc(-n5cccn5)cc4)cc3)c(Cl)cc2N1] | P |
| 2 | 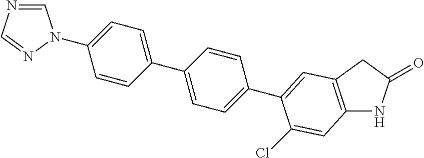 [SMILES: O=C1Cc2cc(-c3ccc(-c4ccc(-n5cncn5)cc4)cc3)c(Cl)cc2N1] | P1 |
| 3 | 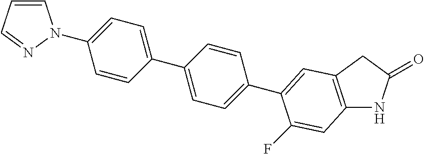 [SMILES: O=C1Cc2cc(-c3ccc(-c4ccc(-n5cccn5)cc4)cc3)c(F)cc2N1] | P2 |
| 4 | 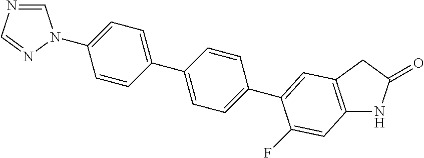 [SMILES: O=C1Cc2cc(-c3ccc(-c4ccc(-n5cncn5)cc4)cc3)c(F)cc2N1] | P3 |
| 5 | 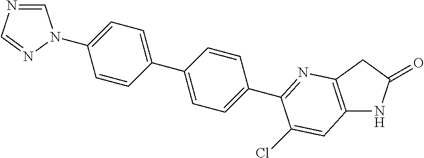 [SMILES: O=C1Cc2nc(-c3ccc(-c4ccc(-n5cncn5)cc4)cc3)c(Cl)cc2N1] | P4 |
| 6 | 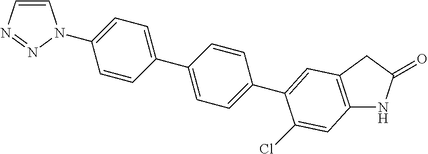 [SMILES: O=C1Cc2cc(-c3ccc(-c4ccc(-n5ccnn5)cc4)cc3)c(Cl)cc2N1] | P5 |
| 7 | 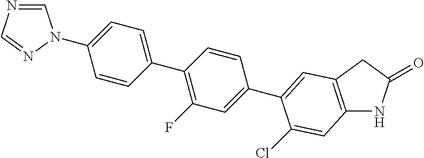 [SMILES: O=C1Cc2cc(-c3ccc(-c4ccc(-n5cncn5)cc4)c(F)c3)c(Cl)cc2N1] | P6 |
| 8 | 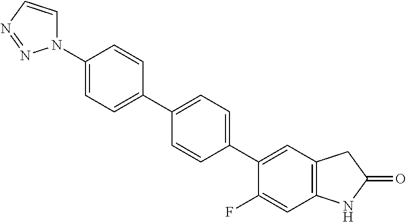 [SMILES: O=C1Cc2cc(-c3ccc(-c4ccc(-n5ccnn5)cc4)cc3)c(F)cc2N1] | P7 |
| 9 | 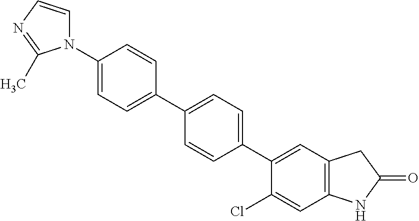 [SMILES: Cc1nccn1-c1ccc(-c2ccc(-c3cc4c(cc3Cl)NC(=O)C4)cc2)cc1] | P8 |
| 10 | 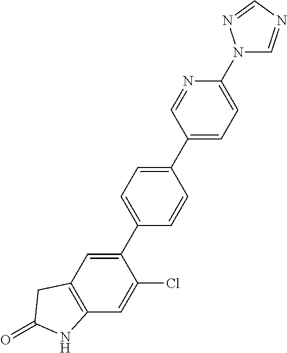 [SMILES: O=C1Cc2cc(-c3ccc(-c4ccc(-n5cncn5)nc4)cc3)c(Cl)cc2N1] | P9 |

The compounds in table 5 were synthesized from the listed intermediates by following the similar procedure described for the Example 8.

TABLE 5
Examples 47 to 71 were prepared by methods analogous to that described for Example 57.

| SI. | | Interme- | |
|---|---|---|---|
| No. | Structure | diates | Physical Data |
| 47 | 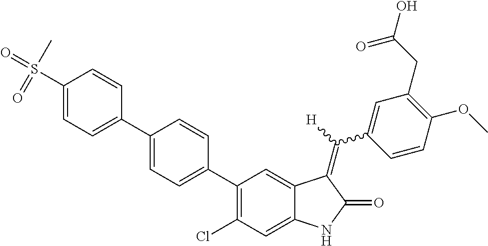 | O and 8 | LCMS: 574.0 (M + H)⁺ HPLC Rt: 14.49 and 15.35 min (Mixture) |
| 48 | 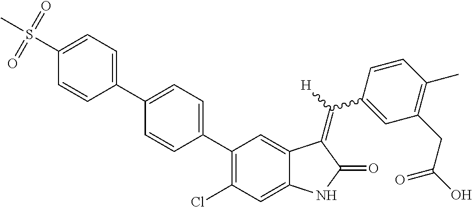 | O and 5 | LCMS: 556.2 (M − H)⁺ HPLC Rt: 14.83 and 15.68 min (Mixture) |
| 49 | 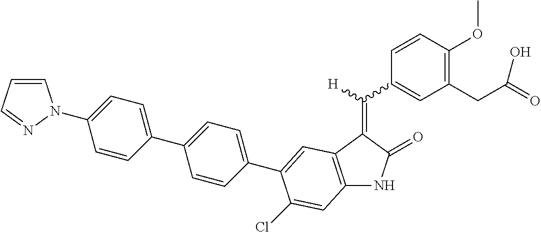 | P and 8 | LCMS: 560.0 (M − H)⁺ HPLC Rt: 16.89 and 17.92 min (Mixture) |
| 50 | 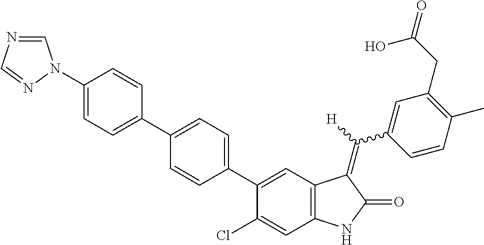 | P1 and 5 | LCMS: 545 (M − H)⁺ HPLC Rt: 15.07 and 16.08 min (Mixture) |
| 51 | 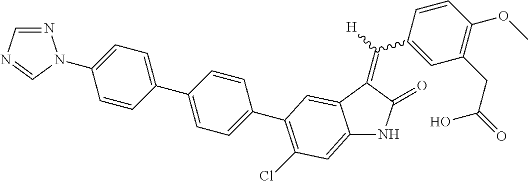 | P1 and 8 | LCMS: 561 (M − H)⁺ HPLC Rt: 14.64 and 15.44 min (Mixture) |
| 52 | 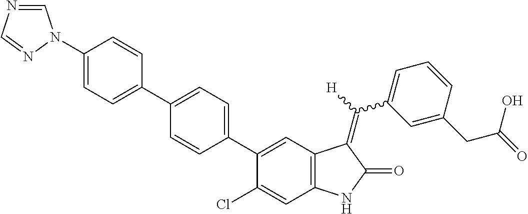 | P1 and 7 | LCMS: 531 (M − H)⁺ HPLC Rt: 14.54 and 15.47 min (Mixture) |
| 53 | 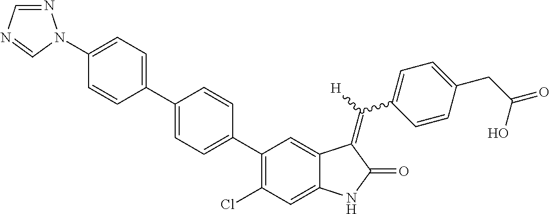 | P1 and 6 | LCMS: 531 (M − H)⁺ HPLC Rt: 14.46 and 15.52 min (Mixture) |
| 54 | 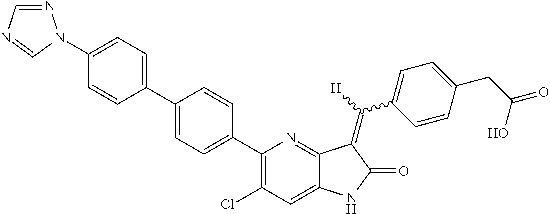 | P4 and 7 | LCMS: 532 (M − H)⁺ HPLC Rt: 14.63 and 15.08 min (Mixture) |
| 55 | 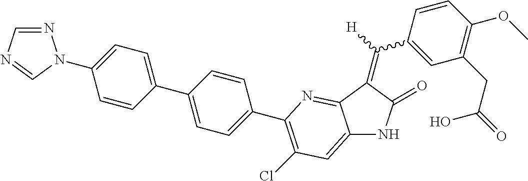 | P4 and 8 | LCMS: 562 (M − H)⁺ HPLC Rt: 15.11 and 15.37 min (Mixture) |
| 56 | 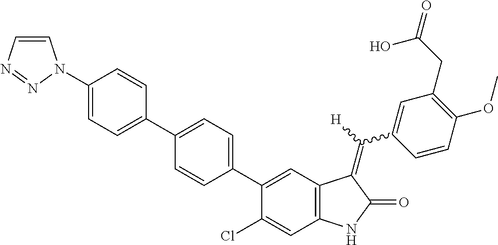 | P5 and 8 | LCMS: 561 (M − H)⁺ HPLC Rt: 14.83 and 15.79 min (Mixture) |
| 57 | 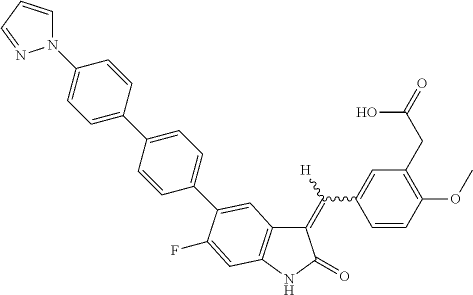 | P2 and 8 | LCMS: 544 (M − H)⁺ HPLC Rt: 16.37 and 17.38 min (Mixture); ¹H NMR (mixture of E/Z, DMSO, 300 MHz) δ 8.52-8.58 (m, 2H), 7.59-8.21 (m, 13H), 7.05-7.1 (t, 1H mixture), 6.75-6.86 (m, 1H), 6.58 (m, 1H), 3.81-3.84 (m, 3H), 3.40-3.47 (m, 2H) |
| 58 | 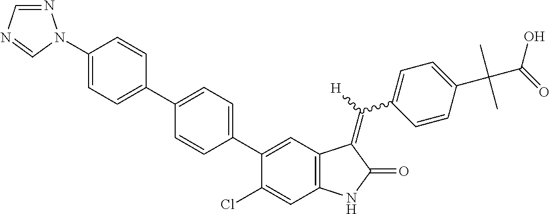 | P1 and 39 | LCMS: 559 (M − H)⁺ HPLC Rt: 15.78 and 16.93 min (Mixture) |
| 59 | 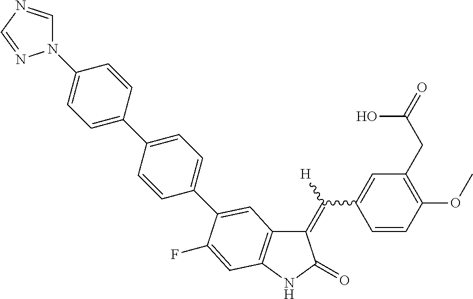 | P3 and 8 | LCMS: 545 (M − H)⁺ HPLC Rt: 14.18 and 15.19 min (Mixture) |
| 60 | 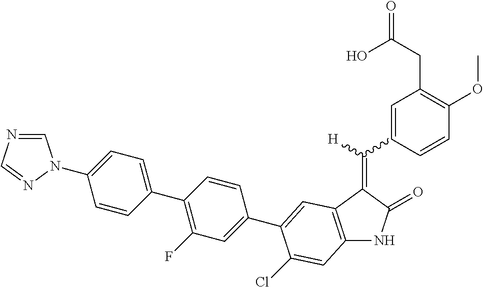 | P6 and 8 | LCMS: 579 (M − H)⁺ HPLC Rt: 14.94 and 16.03 min (Mixture) |
| 61 | 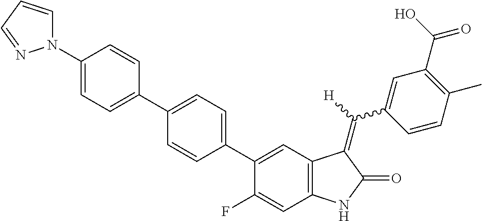 | P2 and 4 | LCMS: 514 (M − H)⁺ HPLC Rt: 17.37 and 17.99 min (Mixture) |
| 63 | 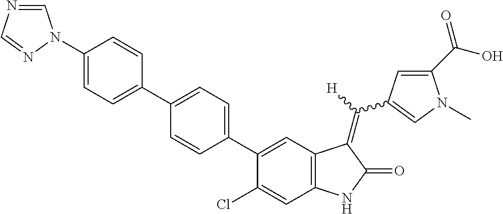 | P1 and 11 | LCMS: 520 (M − H)⁺ HPLC Rt: 13.91 and 15.03 min (Mixture); ¹H NMR: (mixture of E/Z, DMSO, 300 MHz) δ 10.78 (s, 1H), 9.41 (s, 1H), 8.21-8.29 (m, 1H), 7.94-8.02 (m, 4H), 7.83-7.86 (m, 4H), 7.72- 7.77 (d, 2H), 7.57-7.60 (d, 2H), 6.98 (s, 1H), 3.90- 3.94 (m, 3H) |
| 64 | 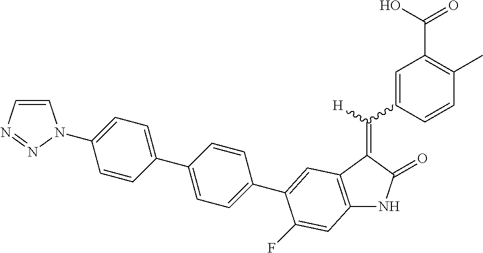 | P7 and 4 | LCMS: 515 (M − H)⁺ HPLC Rt: 15.27 and 15.91 min (Mixture) |
| 65 | 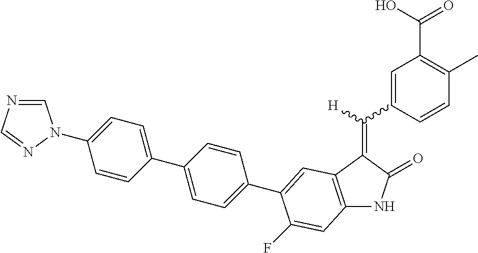 | P3 and 4 | LCMS: 515 (M − H)⁺ HPLC Rt: 15.02 and 15.77 min (Mixture) |
| 66 | 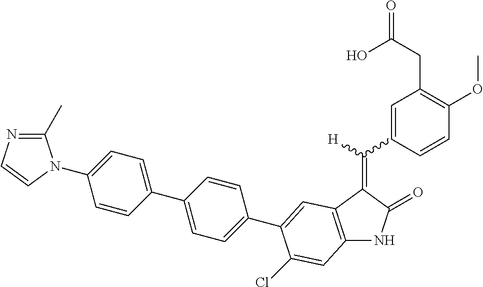 | P8 and 8 | LCMS: 574 (M − H)⁺ HPLC Rt: 10.52 and 11.13 min (Mixture) |
| 69 | 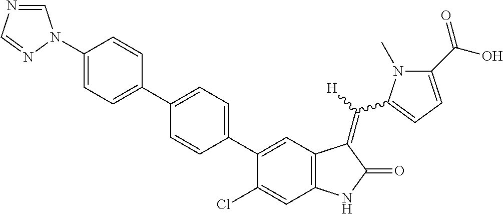 | P1 and 12 | LCMS: 520 (M − H)⁺ HPLC Rt: 14.29 and 15.17 min (Mixture) |
| 70 | 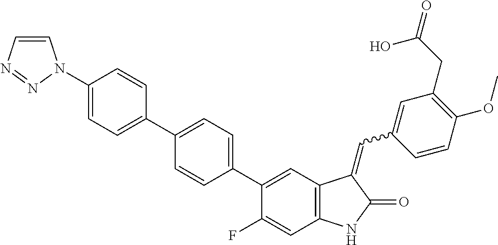 [SMILES: COc1ccc(C=C2C(=O)Nc3cc(F)c(-c4ccc(-c5ccc(-n6ccnn6)cc5)cc4)cc32)cc1CC(=O)O] | P7 and 8 | LCMS: 545 (M − H)⁺ HPLC Rt: 14.41 min |
| 71 | 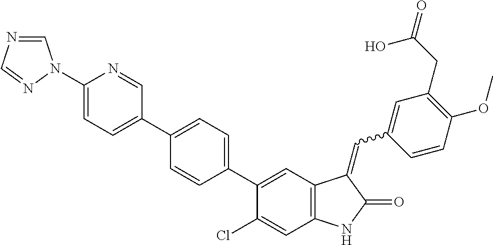 [SMILES: COc1ccc(C=C2C(=O)Nc3cc(Cl)c(-c4ccc(-c5ccc(-n6cncn6)nc5)cc4)cc32)cc1CC(=O)O] | P9 and 8 | LCMS: 563 (M − H)⁺ LC Rt: 2.28 and 2.43 min (Mixture) |

### Representative Procedures for Synthesis of Sodium Salt

### Example 67

### Synthesis of sodium {5-[(E/Z)-{6-chloro-2-oxo-5-[4′-(1H-1,2,4-triazol-1-yl)biphenyl-4-yl]-1,2-dihydro-3H-indol-3-ylidene}methyl]-2-methoxyphenyl}acetate

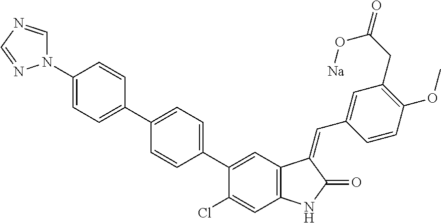

To a stirred solution of acid (compound 67, 1 mmol) in EtOH (25v) was added 0.5M NaOH solution (1 mmol) drop-wise at 30° C. followed by 10V of water. After 30 minutes, excess of ethanol was removed under reduced pressure at 35 deg C. and repeatedly distilled with ethanol two or three times until free solid obtained. The solid was further washed with n-Hexane to obtain compound 67 Compound 67 is soluble in 1 mg/mL water.

### Example 68

### Synthesis of sodium {4-[(E/Z)-{6-chloro-2-oxo-5-[4′-(1H-1,2,4-triazol-1-yl)biphenyl-4-yl]-1,2-dihydro-3H-indol-3-ylidene}methyl]phenyl}acetate

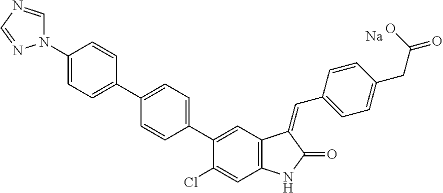

The protocol for synthesizing compound 67 was followed to prepare compound 68. Compound 68 is also soluble in 1 mg/mL water.

### Biological Activity of AMPK

Activation of AMPK by various compounds were measured using in-cell-ELISA for phospho-ACC (Acetyl-CoA carboxylase 1) in HepG2 cells (liver) and fully differentiated myotubes (C2C12 cells) grown in 96 well plate. Cells were treated with 1 μM of the molecule for 2 h in serum free DMEM media. After methanol fixation, cells were blocked with BSA followed by addition of anti-phospho ACC antibody (Cell Signaling) and HRP conjugated secondary antibody. Absorbance values taken at 450 nm and were normalized with total DNA (Hoechest stain). Results were expressed as percentage activation over vehicle control.

| | Activity beta | Activity beta |
|---|---|---|
| | 1 | 2 |
| Example | (at 1 uM) | (at 1 uM) |
| 1 | *** | NT |
| 2 | ** | NT |
| 3 | *** | NT |
| 4 | * | NT |
| 5 | *** | NT |
| 6 | *** | NT |
| 7 | *** | NT |
| 8 | * | NT |
| 9 | *** | NT |
| 10 | * | NT |
| 11 | *** | *** |
| 12 | * | NT |
| 13 | *** | *** |
| 14 | ** | NT |
| 15 | * | NT |
| 16 | * | NT |
| 17 | * | NT |
| 18 | * | NT |
| 19 | *** | NT |
| 20 | * | NT |
| 21 | * | NT |
| 22 | * | NT |
| 23 | * | NT |
| 24 | * | NT |
| 25 | * | NT |
| 26 | * | NT |
| 29 | * | NT |
| 30 | * | NT |
| 31 | * | NT |
| 32 | * | NT |
| 33 | * | NT |
| 34 | * | NT |
| 35 | * | NT |
| 36 | ** | NT |
| 37 | *** | NT |
| 38 | ** | NT |
| 39 | ** | NT |
| 43 | * | NT |
| 44 | * | NT |
| 45 | * | NT |
| 46 | * | NT |
| 48 | *** | *** |
| 49 | *** | NT |
| 50 | ** | NT |
| 51 | *** | *** |
| 52 | *** | NT |
| 53 | *** | *** |
| 56 | *** | *** |
| 59 | *** | NT |
| 60 | *** | NT |
| 61 | *** | NT |
| 63 | *** | NT |
| 64 | *** | NT |
| 65 | *** | NT |
| 66 | * | NT |
| 67 | *** | NT |
| 68 | *** | NT |
| 69 | *** | NT |

* <20
** <20 > 40
*** >40
NT not tested

It is understood that the foregoing detailed description and accompanying examples are merely illustrative and are not to be taken as limitations upon the scope of the invention. Various changes and modifications to the disclosed aspects will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

1. A compound of formula I 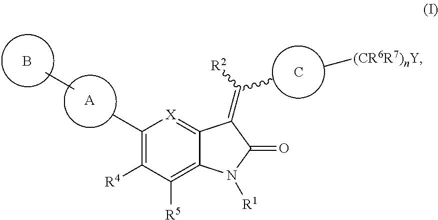
wherein
ring A, ring B and ring C are each independently selected from the group consisting of optionally substituted C₆-C₁₈aryl and optionally substituted C₁-C₁₈heteroaryl;
X is selected from the group consisting of N and CR³;
Y is selected from the group consisting of H and COR⁸;
R¹ and R² are each independently selected from the group consisting of H and optionally substituted C₁-C₆ alkyl;
R³ and R⁵ are each independently selected from the group consisting of H, halogen, CN, —NO₂, SH, CF₃, OH, CO₂H, CONH₂, OCF₃, and optionally substituted C₁-C₁₂alkyl;
R⁴ is selected from the group consisting of H, F, Cl, Br and I;
R⁶ and R⁷ are each independently selected from the group consisting of H and optionally substituted C₁-C₆ alkyl;
R⁸ is selected from the group consisting of H, OH, optionally substituted C₁-C₆ alkyl and —NR⁹R¹⁰;
wherein R⁹ and R¹⁰ are each independently selected from the group consisting of H and optionally substituted C₁-C₆ alkyl, or R⁹ and R¹⁰ when taken together to the nitrogen atom to which they are attached form an optionally substituted C₂-C₁₂ heterocycloalkyl group,
n is an integer selected from the group consisting of 0, 1 and 2;
wherein the term “optionally substituted” used within the definitions hereinbefore is not limited to but preferably means 1, 2 or 3 optional substituents independently selected from F, Cl, Br, I, CH₃, CH₂CH₃, CH(CH₃)₂, C(CH₃)₃, OH, OCH₃, OCH₂CH₃, OCH(CH₃)₂, OC(CH₃)₃, CF₃, OCF₃, NO₂, SO₃H, SO₂CH₃, NH₂, NHCH₃, N(CH₃)₂ and CN;
or a pharmaceutically acceptable salt, N-oxide, or prodrug thereof.

2. A compound according to claim 1, wherein Y is COR⁸, R⁸ is selected from the group consisting of H, OH and NR⁹R¹⁰, wherein R⁹ and R¹⁰ are defined as in claim 1,
or a pharmaceutically acceptable salt thereof.

3. A compound according to claim 2, wherein Y is COR⁸, and R⁸ is OH, providing compounds of substructure formula (Ia) 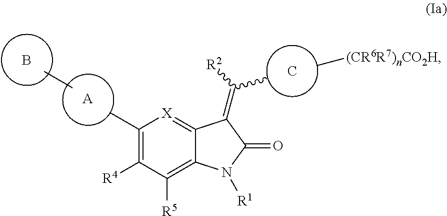
wherein
ring A, ring B and ring C are each independently selected from the group consisting of optionally substituted C₆-C₁₈aryl and optionally substituted C₁-C₁₈heteroaryl;
X is selected from the group consisting of N and CR³;
R¹ and R² are each independently selected from the group consisting of H and optionally substituted C₁-C₆ alkyl;
R³ and R⁵ are each independently selected from the group consisting of H, halogen, CN, —NO₂, SH, CF₃, OH, CO₂H, CONH₂ OCF₃, and optionally substituted C₁-C₁₂alkyl;
R⁴ is selected from the group consisting of H, F, Cl, Br and I;
R⁶ and R⁷ are each independently selected from the group consisting of H and optionally substituted C₁-C₆ alkyl;
n is an integer selected from the group consisting of 0, 1 and 2;
or a pharmaceutically acceptable salt thereof.

4. A compound according to claim 1, wherein X is N,
or a pharmaceutically acceptable salt thereof.

5. A compound according to claim 1, wherein X is CR³ and R³ is selected from the group consisting of H, halogen, CN, —NO₂, SH, CF₃, OH, CO₂H, CONH₂, OCF₃, and optionally substituted but preferably unsubstituted C₁-C₁₂alkyl,
or a pharmaceutically acceptable salt thereof.

6. A compound according to claim 3, wherein X is N, providing compounds of substructure formula (Ib) 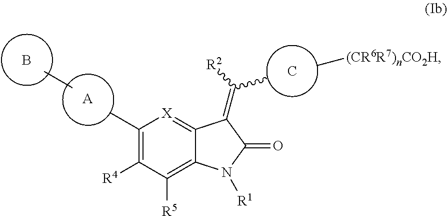
or a pharmaceutically acceptable salt thereof.

7. A compound according to claim 3, wherein X is CR³ and R³ is H, providing compounds of substructure formula (Ic) 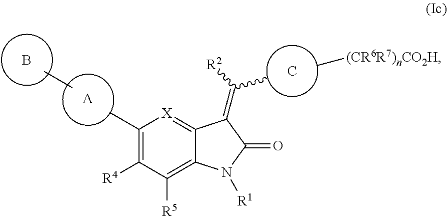
or a pharmaceutically acceptable salt thereof.

8. A compound according to claim 1, wherein R¹ and R² are independently selected from the group consisting of CH₃, CH₂CH₃, CH(CH₃)₂ and C(CH₃)₃,
or a pharmaceutically acceptable salt thereof.

9. A compound according to claim 7, wherein R¹ is H, R² is H, and R⁵ is H, providing compounds of substructure formula (Id) 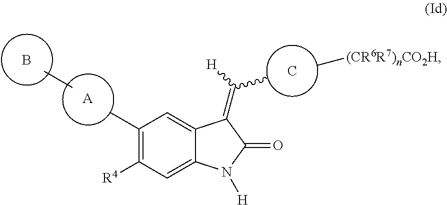
or a pharmaceutically acceptable salt thereof.

10. A compound according to claim 1, wherein ring A is selected from the group consisting of: 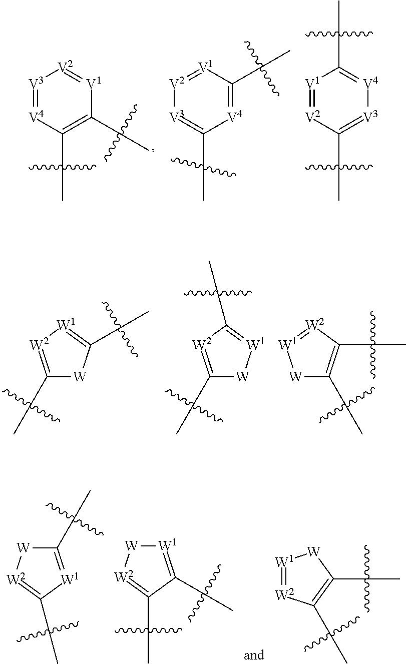
wherein V¹, V², V³ and V⁴ are each independently selected from the group consisting of N, and C(R¹¹);
W is selected from the group consisting of O, S and NR¹¹;
W¹ and W² are each independently selected from the group consisting of N and CR¹¹;
wherein each R¹¹ is independently selected from the group consisting of H, halogen, OH, NO₂, CN, SH, NH₂, CF₃, OCF₃, optionally substituted C₁-C₁₂alkyl, optionally substituted C₁-C₁₂haloalkyl, optionally substituted C₂-C₁₂alkenyl, optionally substituted C₂-C₁₂haloalkenyl optionally substituted C₂-C₁₂alkynyl, optionally substituted C₂-C₁₂haloalkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₂heteroaryloxy, optionally substituted C₁-C₁₂alkylamino, SR¹¹¹, SO₃H, SO₂NR¹¹¹R¹¹², SO₂R¹¹¹, SONR¹¹¹R¹¹², SOR¹¹¹, COR¹¹¹, COOH, COOR¹¹¹, CONR¹¹¹R¹¹², NR¹¹¹COR¹¹², NR¹¹¹COOR¹¹², NR¹¹¹SO₂R¹¹², NR¹¹¹CONR¹¹²R¹¹³, NR¹¹¹R¹¹², and acyl;
each R¹¹¹, R¹¹² and R¹¹³ is independently selected from the group consisting of H, optionally substituted C₁-C₁₂alkyl, optionally substituted C₂-C₁₀heteroalkyl, optionally substituted C₁-C₁₂haloalkyl, optionally substituted C₃-C₁₂cycloalkyl, optionally substituted C₆-C₁₈aryl, and optionally substituted C₁-C₁₈heteroaryl.
but wherein preferably each R¹¹ is independently selected from the group consisting of OH, F, Br, Cl, methyl, CN, NO₂, SH, CO₂H, CONH₂, OCF₃, trifluoromethyl, ethyl, 2,2,2-trifluoroethyl, isopropyl, propyl, 2-ethyl-propyl, 3,3-dimethyl-propyl, butyl, isobutyl, 3,3-dimethyl-butyl, 2-ethyl-butyl, pentyl, 2-methyl-pentyl, pent-4-enyl, hexyl, heptyl, octyl, phenyl, NH₂, phenoxy, hydroxy, methoxy, ethoxy, pyrrol-1-yl, and 3,5-dimethyl-pyrazol-1-yl,
or a pharmaceutically acceptable salt thereof.

11. A compound according to claim 1, wherein ring A is an optionally substituted C₆-C₁₈aryl group of the formula (II) 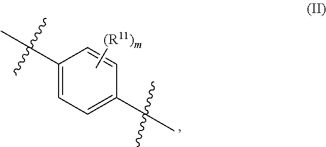
wherein each R¹¹ is independently selected from the group consisting of H, halogen, CN, OH, NH₂, NO₂, SH, CF₃, CO₂H, CONH₂, C₁-C₁₂alkyl, C₁-C₁₂haloalkyl, C₁-C₁₂alkoxyl, and C₁-C₁₂haloalkoxyl,
but preferably R¹¹ is independently selected from the group consisting of H, halogen, CN, —NO₂, SH, CF₃, OH, CO₂H, CONH₂, OCF₃, and optionally substituted C₁-C₁₂alkyl; and
m is an integer selected from the group consisting of 0, 1, 2, 3, and 4,
or a pharmaceutically acceptable salt thereof.

12. A compound according to claim 9, wherein ring A is an optionally substituted C₆-C₁₈aryl group of the formula (II) 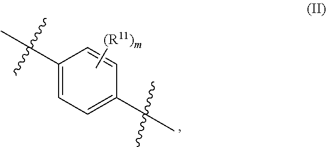
wherein each R¹¹ is independently selected from the group consisting of H, halogen, CN, OH, NH₂, NO₂, SH, CF₃, CO₂H, CONH₂, C₁-C₁₂alkyl, C₁-C₁₂haloalkyl, C₁-C₁₂alkoxyl, and C₁-C₁₂haloalkoxyl,
but preferably R¹¹ is independently selected from the group consisting of H, halogen, CN, —NO₂, SH, CF₃, OH, CO₂H, CONH₂, OCF₃, and optionally substituted C₁-C₁₂alkyl; and
m is an integer selected from the group consisting of 0, 1, 2, 3, and 4,
providing compounds of substructure formula (Ie) 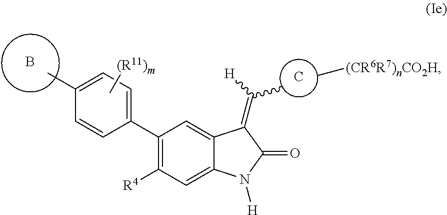
wherein
ring B and ring C are each independently selected from the group consisting of optionally substituted C₆-C₁₈aryl and optionally C₁-C₁₈heteroaryl;
R⁴ is selected from the group consisting of H, F, Cl, Br and I;
R⁶ and R⁷ are each independently selected from the group consisting of H and optionally substituted C₁-C₆ alkyl;
n is an integer selected from the group consisting of 0, 1 and 2;
or a pharmaceutically acceptable salt thereof.

13. A compound according to claim 1, wherein ring B is selected from the group consisting of: 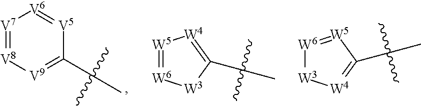
wherein V⁵, V⁶, V⁷, V⁸ and V⁹ are each independently selected from the group consisting of N, and C(R¹²);
W³ is selected from the group consisting of O, S and NR¹²;
W⁴, W⁵, and W⁶ are each independently selected from the group consisting of N and CR¹²;
wherein each R¹² is independently selected from the group consisting of H, halogen, OH, NO₂, CN, SH, NH₂, CF₃, OCF₃, optionally substituted C₁-C₁₂alkyl, optionally substituted C₁-C₁₂haloalkyl, optionally substituted C₂-C₁₂alkenyl, optionally substituted C₂-C₁₂haloalkenyl optionally substituted C₂-C₁₂alkynyl, optionally substituted C₂-C₁₂haloalkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₂heteroaryloxy, optionally substituted C₁-C₁₂alkylamino, SR¹³, SO₃H, SO₂NR¹³R¹⁴, SO₂R¹³, SONR¹³R¹⁴, SOR¹³, COR¹⁴, COOH, COOR¹³, CONR¹⁴R¹⁵, NR¹⁴COR¹⁵, NR¹⁴COOR¹⁵, NR¹⁴SO₂R¹⁵, NR¹³CONR¹⁴R¹⁵, NR¹⁴R¹⁵, and acyl;
each R¹³, R¹⁴ and R¹⁵ is independently selected from the group consisting of H, optionally substituted C₁-C₁₂alkyl, optionally substituted C₂-C₁₀heteroalkyl, optionally substituted C₁-C₁₂haloalkyl, optionally substituted C₃-C₁₂cycloalkyl, optionally substituted C₆-C₁₈aryl, and optionally substituted C₁-C₁₈heteroaryl;
but preferably R¹² is independently selected from the group consisting of OH, F, Br, Cl, methyl, CN, NO₂, SH, CO₂H, CONH₂, OCF₃, trifluoromethyl, ethyl, 2,2,2-trifluoroethyl, isopropyl, propyl, 2-ethyl-propyl, 3,3-dimethyl-propyl, butyl, isobutyl, 3,3-dimethyl-butyl, 2-ethyl-butyl, pentyl, 2-methyl-pentyl, pent-4-enyl, hexyl, heptyl, octyl, phenyl, NH₂, phenoxy, hydroxy, methoxy, ethoxy, pyrrol-1-yl, and 3,5-dimethyl-pyrazol-1-yl,
or a pharmaceutically acceptable salt thereof.

14. A compound according to claim 1, wherein ring B is an optionally substituted C₆-C₁₈aryl group of the formula (III) 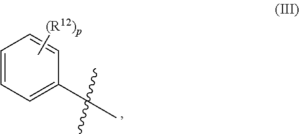
wherein each R¹² is independently selected from the group consisting of H, halogen, OH, NO₂, CN, SH, NH₂, CF₃, OCF₃, optionally substituted C₁-C₁₂alkyl, optionally substituted C₁-C₁₂haloalkyl, optionally substituted C₂-C₁₂alkenyl, optionally substituted C₂-C₁₂haloalkenyl optionally substituted C₂-C₁₂alkynyl, optionally substituted C₂-C₁₂haloalkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₂heteroaryloxy, optionally substituted C₁-C₁₂alkylamino, SR¹³, SO₃H, SO₂NR¹³R¹⁴, SO₂R¹³, SONR¹³R¹⁴, SOR¹³, COR¹⁴, COOH, COOR¹³, CONR¹⁴R¹⁵, NR¹⁴COR¹⁵, NR¹⁴COOR¹⁵, NR¹⁴SO₂R¹⁵, NR¹³CONR¹⁴R¹⁵, NR¹⁴R¹⁵, and acyl;
but preferably R¹² is independently selected from the group consisting of H, halogen, CN, —NO₂, SH, CF₃, OH, CO₂H, CONH₂, OCF₃, and optionally substituted C₁-C₁₂alkyl;
each R¹³, R¹⁴ and R¹⁵ independently selected from the group consisting of H, optionally substituted C₁-C₁₂alkyl, optionally substituted C₂-C₁₀heteroalkyl, optionally substituted C₁-C₁₂haloalkyl, optionally substituted C₃-C₁₂cycloalkyl, optionally substituted C₆-C₁₈aryl, and optionally substituted C₁-C₁₈heteroaryl;
p is an integer selected from the group consisting of 0, 1, 2, 3, 4 and 5;
wherein in a preferred embodiment p is 1 and R¹² is an optionally substituted C₁-C₁₈heteroaryl selected from the group consisting of 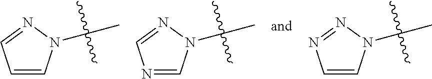 [SMILES: *n1cccn1 ; *n1cncn1 ; *n1ccnn1]
wherein each optional substituent is independently selected from the group consisting of F, Cl, Br, I, CH₃, CH₂CH₃, CH(CH₃)₂, C(CH₃)₃, OH, OCH₃, OCH₂CH₃, OCH(CH₃)₂, OC(CH₃)₃, CF₃, OCF₃, NO₂, SO₃H, SO₂CH₃, NH₂, NHCH₃, N(CH₃)₂ and CN,
or a pharmaceutically acceptable salt thereof.

15. A compound according to claim 12, wherein ring B is an optionally substituted C₆-C₁₈aryl group of the formula (III) 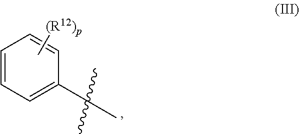
wherein each R¹² is independently selected from the group consisting of H, halogen, OH, NO₂, CN, SH, NH₂ CF₃ OCF₃, optionally substituted C₁-C₁₂alkyl, optionally substituted C₁-C₁₂haloalkyl, optionally substituted C₂-C₁₂alkenyl, optionally substituted C₂-C₁₂haloalkenyl optionally substituted C₂-C₁₂alkynyl, optionally substituted C₂-C₁₂haloalkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₂heteroaryloxy, optionally substituted C₁-C₁₂alkylamino, SR¹³, SO₃H, SO₂NR¹³R¹⁴, SO₂R¹³, SONR¹³R¹⁴, SOR¹³, COR¹⁴, COOH, COOR¹³, CONR¹⁴R¹⁵, NR¹⁴COR¹⁵, NR¹⁴COOR¹⁵, NR¹⁴SO₂R¹⁵, NR¹³CONR¹⁴R¹⁵, NR¹⁴R¹⁵, and acyl;
but preferably R¹² is independently selected from the group consisting of H, halogen, CN, —NO₂, SH, CF₃, OH, CO₂H, CONH₂ OCF₃, and optionally substituted C₁-C₁₂alkyl;
each R¹³, R¹⁴ and R¹⁵ is independently selected from the group consisting of H, optionally substituted C₁-C₁₂alkyl, optionally substituted C₂-C₁₀heteroalkyl optionally substituted C₁-C₁₂haloalkyl, optionally substituted C₃-C₁₂cycloalkyl, optionally substituted C₆-C₁₈aryl, and optionally substituted C₁-C₁₈heteroaryl;
p is an integer selected from the group consisting of 0, 1, 2, 3, 4 and 5;
wherein in a preferred embodiment p is 1 and R¹² is an optionally substituted C₁-C₁₈heteroaryl selected from the group consisting of 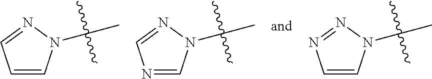 [SMILES: *n1cccn1 ; *n1cncn1 ; *n1ccnn1]
wherein each optional substituent is independently selected from the group consisting of F, Cl, Br, I, CH₃ CH₂CH₃, CH(CH₃)₂, C(CH₃)₃, OH, OCH₃ OCH₂CH₃, OCH(CH₃)₂, OC(CH₃)₃, CF₃, OCF₃, NO₂, SO₃H, SO₂CH₃, NH₂, NHCH₃, N(CH₃)₂ and CN, providing compounds of substructure formula (If) 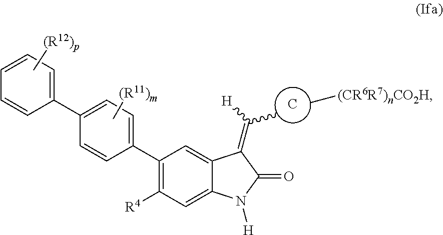
wherein each R¹¹ is independently selected from the group consisting of H, halogen, CN, OH, NH₂, NO₂, SH, CF₃, CO₂H, CONH₂, C₁-C₁₂alkyl, C₁-C₁₂haloalkyl, C₁-C₁₂alkoxyl, and C₁-C₁₂haloalkoxyl,
but preferably R¹¹ is independently selected from the group consisting of H, halogen, CN, —NO₂, SH, CF₃, OH, CO₂H, CONH₂, OCF₃, and optionally substituted C₁-C₁₂alkyl; and
m is an integer selected from the group consisting of 0, 1, 2, 3, and 4,
and wherein ring C are each independently selected from the group consisting of optionally substituted C₆-C₁₈aryl and optionally substituted C₁-C₁₈heteroaryl;
R⁴ is selected from the group consisting of H, F, Cl, Br and I;
R⁶ and R⁷ are each independently selected from the group consisting of H and optionally substituted C₁-C₆ alkyl;
n is an integer selected from the group consisting of 0, 1 and 2;
or a pharmaceutically acceptable salt thereof.

16. A compound according to claim 15, wherein p is 1 and R¹² is OH located at the ortho position, providing compounds of substructure formula (Ifa) 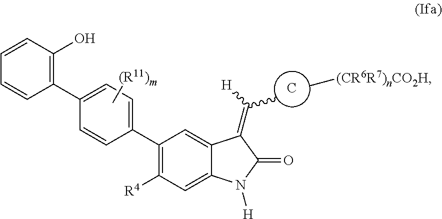
or a pharmaceutically acceptable salt thereof.

17. A compound according to claim 1, wherein ring C is selected from the group consisting of: 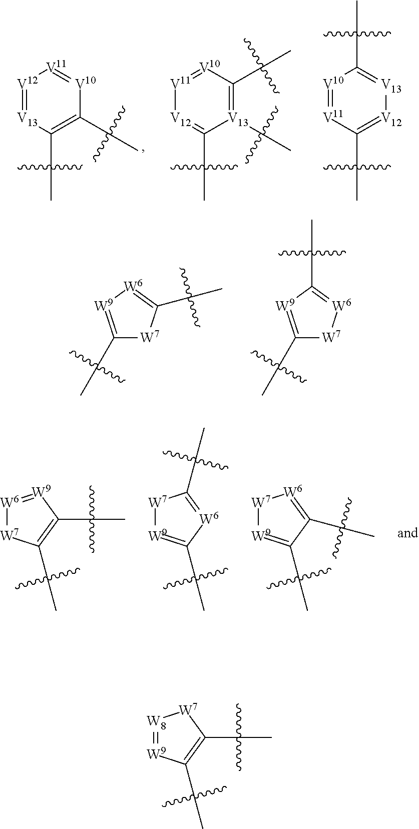
wherein V¹⁰, V¹¹, V¹² and V¹³ are each independently selected from the group consisting of N, and C(R¹⁶);
W⁷ is selected from the group consisting of O, S and NR¹⁶;
W⁸ and W⁹ are each independently selected from the group consisting of N and CR¹⁶;
wherein each R¹⁶ is independently selected from the group consisting of H, halogen, OH, NO₂, CN, SH, NH₂, CF₃, OCF₃, optionally substituted C₁-C₁₂alkyl, optionally substituted C₁-C₁₂haloalkyl, optionally substituted C₂-C₁₂alkenyl, optionally substituted C₂-C₁₂haloalkenyl optionally substituted C₂-C₁₂alkynyl, optionally substituted C₂-C₁₂haloalkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₂heteroaryloxy, optionally substituted C₁-C₁₂alkylamino, SR¹⁶¹, SO₃H, SO₂NR¹⁶¹R¹⁶², SO₂R¹⁶¹, SONR¹⁶¹R¹⁶², SOR¹⁶¹, COR¹⁶¹, COOH, COOR¹⁶¹, CONR¹⁶¹R¹⁶², NR¹⁶¹COR¹⁶², NR¹⁶¹COOR¹⁶², NR¹⁶¹SO₂R¹⁶², NR¹⁶¹CONR¹⁶²R¹⁶³, NR¹⁶¹R¹⁶², and acyl;
each R¹⁶¹, R¹⁶² and R¹⁶³ is independently selected from the group consisting of H, optionally substituted C₁-C₁₂alkyl, optionally substituted C₂-C₁₀heteroalkyl, optionally substituted C₁-C₁₂haloalkyl, optionally substituted C₃-C₁₂cycloalkyl, optionally substituted C₆-C₁₈aryl, and optionally substituted C₁-C₁₈heteroaryl;
wherein each optional substituent is independently selected from the group consisting of F, Cl, Br, I, CH₃, CH₂CH₃, CH(CH₃)₂, C(CH₃)₃, OH, OCH₃, OCH₂CH₃, OCH(CH₃)₂, OC(CH₃)₃, CF₃, OCF₃, NO₂, SO₃H, SO₂CH₃, NH₂, NHCH₃, N(CH₃)₂ and CN,
or a pharmaceutically acceptable salt thereof.

18. A compound according to claim 1, wherein ring C is an optionally substituted C₆-C₁₈aryl group of the formula (IV) 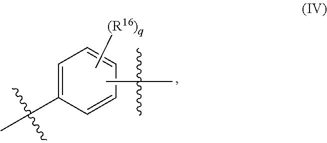
wherein each R¹⁶ is independently selected from the group consisting of H, halogen, CN, —NO₂, SH, CF₃, OH, CO₂H, CONH₂, OCF₃, C₁-C₁₂alkyl and OC₁-C₁₂alkyl; and
q is an integer selected from the group consisting of 0, 1, 2, 3, and 4,
or a pharmaceutically acceptable salt thereof.

19. A compound according to claim 15, wherein ring C is an optionally substituted C₆-C₁₈aryl group of the formula (IV) 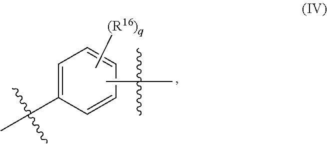
wherein each R¹⁶ is independently selected from the group consisting of H, halogen, CN, —NO₂, SH, CF₃, OH, CO₂H, CONH₂, OCF₃, C₁-C₁₂alkyl and OC₁-C₁₂alkyl; and
q is an integer selected from the group consisting of 0, 1, 2, 3, and 4,
providing compounds of substructure formula (Ig) 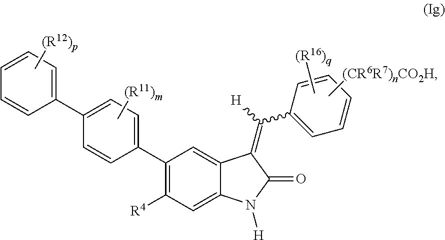
wherein each R¹² is independently selected from the group consisting of H, halogen, OH, NO₂, CN, SH, NH₂, CF₃, OCF₃, optionally substituted C₁-C₁₂alkyl, optionally substituted C₁-C₁₂haloalkyl, optionally substituted C₂-C₁₂alkenyl, optionally substituted C₂-C₁₂haloalkenyl optionally substituted C₂-C₁₂alkynyl, optionally substituted C₂-C₁₂haloalkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₂heteroaryloxy, optionally substituted C₁-C₁₂alkylamino, SR¹³, SO₃H, SO₂NR¹³R¹⁴, SO₂R¹³, SONR¹³R¹⁴, SOR¹³, COR¹⁴, COOH, COOR¹³, CONR¹⁴R¹⁵, NR¹⁴COR¹⁵, NR¹⁴COOR¹⁵, NR¹⁴SO₂R¹⁵, NR¹³CONR¹⁴R¹⁵, NR¹⁴R¹⁵, and acyl;
but preferably R¹² is independently selected from the group consisting of H, halogen, CN, —NO₂, SH, CF₃, OH, CO₂H, CONH₂ OCF₃, and optionally substituted C₁-C₁₂alkyl;
each R¹³, R¹⁴ and R¹⁵ is independently selected from the group consisting of H, optionally substituted C₁-C₁₂alkyl, optionally substituted C₂-C₁₀heteroalkyl, optionally substituted C₁-C₁₂haloalkyl, optionally substituted C₃-C₁₂cycloalkyl, optionally substituted C₆-C₁₈aryl, and optionally substituted C₁-C₁₈heteroaryl;
p is an integer selected from the group consisting of 0, 1, 2, 3, 4 and 5;
wherein in a preferred embodiment p is 1 and R¹² is an optionally substituted C₁-C₁₈heteroaryl selected from the group consisting o 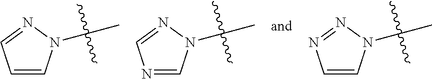 [SMILES: *n1cccn1 ; *n1cncn1 ; *n1ccnn1]
wherein each optional substituent is independently selected from the group consisting of F, Cl, Br, I, CH₃ CH₂CH₃, CH(CH₃)₂, C(CH₃)₃, OH, OCH₃, OCH₂CH₃, OCH(CH₃)₂, OC(CH₃)₃, CF₃, OCF₃, NO₂, SO₃H, SO₂CH₃, NH₂, NHCH₃, N(CH₃)₂ and CN, and wherein each R¹⁶ is independently selected from the group consisting of H, halogen, CN, —NO₂, SH, CF₃, OH, CO₂H, CONH₂, OCF₃, C₁-C₁₂alkyl and OC₁-C₁₂alkyl; and
q is an integer selected from the group consisting of 0, 1, 2, 3, and 4,
or a pharmaceutically acceptable salt thereof.

20. A pharmaceutical composition comprising one or more compounds according to claim 1, optionally together with one or more inert carriers and/or diluents.

21. A pharmaceutical composition according to claim 20 and one or more additional therapeutic agents, optionally together with one or more inert carriers and/or diluents.

22. A pharmaceutical composition according to claim 20 and one additional therapeutic agent selected from the group consisting of antidiabetic agents, agents for the treatment of overweight and/or obesity and agents for the treatment of high blood pressure, heart failure and/or atherosclerosis, optionally together with one or more inert carriers and/or diluents.

23. A method for treating diseases or conditions which can be influenced by the modulation of the function of AMP-activated protein kinase (AMPK), particularly, for the prophylaxis and/or therapy of metabolic diseases, such as diabetes, more specifically type 2 diabetes mellitus, and conditions associated with the disease, including insulin resistance, obesity, cardiovascular disease and dyslipidemia, comprising administering a compound of claim 1 to a patient in need thereof.

24. (canceled)

25. (canceled)
